(19) United States
(12) Patent Application Publication
(10) Pub. No.: US 2017/0321281 A1
(43) Pub. Date: Nov. 9, 2017

(54) METHODS AND COMPOSITIONS FOR TREATMENT OF GLIOBLASTOMA

(71) Applicant: The Trustees Of Columbia University in the City of New York, New York, NY (US)

(72) Inventors: ANTONIO IAVARONE, New York, NY (US); RAUL RABADAN, New York, NY (US); GAETANO FINOCCHIARO, Milano (IT); DO-HYUN NAM, Seoul (KR)

(21) Appl. No.: 15/496,869

(22) Filed: Apr. 25, 2017

Related U.S. Application Data
(60) Provisional application No. 62/327,284, filed on Apr. 25, 2016.

(51) Int. Cl. C12Q 1/68

(52) U.S. Cl. CPC C12Q 1/6886; C12Q 2600/106; C12Q 2600/156

(57) ABSTRACT

Techniques for treating a subject with M-GBM and recurrent GBM are provided. Example methods include obtaining at least two M-GBM tumor samples from different locations within a patient, extracting genomic DNA from each of the tumor samples, and determining whether the subject has a mutation in PI3K-AKT-mTOR (PAM) pathway in both DNA samples. If a mutation in PAM pathway is present in each of the isolated DNA samples, the method can further include treating the subject with an effective amount of an agent that inhibits the PAM pathway. Pharmaceutical agents and kits for use in the treatment of M-GBM and recurrent GBM are also provided.

### CROSS REFERENCE TO RELATED APPLICATIONS

The present application claims priority to U.S. Provisional Application No. 62/327,284, filed Apr. 25, 2016, the contents of which are hereby incorporated by reference in its entirety.

### STATEMENT OF GOVERNMENT INTEREST

This invention was made with government support National Institutes of Health under Grant Nos. U54 CA193313, R01 CA185486, R01 CA179044, R01 CA178546, R01 NS061776, F99 CA212478, and T32 CA09503. The Government has certain rights in the invention.

### SEQUENCE LISTING

The instant application contains a Sequence Listing which has been submitted electronically in ASCII format and is hereby incorporated by reference in its entirety. The ASCII copy, created on Apr. 25, 2017, is named 070050_5898_SL.txt and is 42,366 bytes in size.

### BACKGROUND

Glioblastoma (GBM) is an aggressive and the most common primary malignancy of the central nervous system. GBMs are tumors of astrocytes, which are star-shaped glial cells that form the neuronal network and perform a variety of important, active roles in the brain. GBMs are frequently malignant and are generally found in the cerebral hemispheres of the brain. At the time of filing this disclosure, the pathogenesis of GBM is not completely understood and patients diagnosed with GBM have poor prognosis and survival rate, with a median survival of 12-15 months from diagnosis. Rare inherited cancer syndrome, ionizing radiation, and occupational chemical exposures are known risk factors associated with GBMs.

GBMs usually appear as a single peripherally enhancing lesion on imaging, but sometimes appear as multiple foci of enhancing lesions. It is termed multifocal GBM if there is a macroscopic and/or microscopic connection between enhancing lesions. If there is no communication between lesions, it is termed multicentric GBM. Multifocal/multicentric GBMs (M-GBMs) have been shown to have worse prognosis than solitary GBM.

Despite numerous clinical trials, GBMs remain a challenging form of cancer to treat. Certain conventional treatment includes surgery followed by radiotherapy with concomitant and adjuvant temozolomide-based chemotherapy. However, the malignant nature of GBMs often results in resistance to standard treatment and a high recurrence rate. Molecular targeted therapy can be a treatment option for GBMs, although the intrinsic heterogeneity present in GBMs can present a challenge. Therefore there remains a need in the art to develop a more efficient treatment strategy, especially for M-GBMs.

### SUMMARY OF THE INVENTION

The presently disclosed subject matter provides techniques for treating a subject with M-GBM including determining whether the subject has a mutation in PI3K-AKT-mTOR (PAM) pathway and treating the subject with an effective amount of a PAM pathway inhibiting agent.

In certain aspects, methods for treating a subject with M-GBM are provided. Methods can include obtaining at least two M-GBM tumor samples from different locations within a subject, extracting genomic DNA from each of theat least two tumor samples to obtain at least two corresponding extracted genomic DNA samples, and determining whether the subject has a PAM pathway mutation in each of the at least two extracted genomic DNA samples. If a PAM pathway mutation is determined in each of the at least two DNA samples, the method can further include treating the subject with an effective amount of a PAM pathway inhibiting agent.

As embodied herein, example methods can include treating a subject with a gain-of-function mutation in the PAM pathway in M-GBM, and treating a subject with a mutation in PIK3CA gene. For example and embodied herein, the mutation can be at amino acid 4, 364, 1016, 1035, 1043 of PIK3CA protein, or at equivalent positions of homologous sequences thereto. The PIK3CA mutation can also be selected from the group consisting of R4Q, G364R, F1016C, A1035V, M1043I, and M1043V. The method further includes treating a subject with a mutation in one or more of AKT1, AKT2, AKT3, and/or mTOR genes.

As embodied herein, and without limitation, the PAM pathway inhibiting agent can be selected from the group consisting of BKM120 (Buparlisib), XL147 (Pilaralisib), GDC0941 (Pictilisib), BYL719 (Alpelisib), GDC0032 (Tazelisib), NVP-BEZ235, LY3023414, GSK2126458, BEZ235, PF-05212384 (PKI-587), AZD5363, MK-2206, GSK21411795 (Uprosertib), GDC-0068 (Lpatasertib), LNK128, AZD2014, AZD8055, MLN0138, CC-223, RAD001 (Everolimus), rapamycin (Sirolimus), CCI-779 (Temsirolimus), AP23573 (Ridaforolimus), and combinations thereof.

As embodied herein, and without limitation, the PAM pathway inhibiting agent can be administered orally. Alternatively or additionally, the agent that inhibits the PAM pathway can be administered intravenously.

As embodied herein, the PAM pathway inhibiting agent can include a nucleic acid that specifically binds to a nucleic acid encoding PIK3CA, and reduces PI3K expression and/or activity. The agent can include a microRNA (miRNA) molecule, small interfering RNA (siRNA) molecule, short hairpin RNA (shRNA) molecule, catalytic RNA molecule, and/or catalytic DNA molecule.

In certain aspects, methods for treating M-GBM in a subject can include administering, to the subject, an effective amount of a PAM pathway inhibiting agent. As embodied herein, the method can further include administering to the subject an additional therapeutic agent, a stabilizing compound, and/or a biocompatible pharmaceutical carrier.

The presently disclosed subject matter also provides kits. As embodied herein, an example kit for determining the prognosis of a subject with multifocal/multicentric glioblastoma (M-GBM) by determining the presence of a PI3K-AKT-mTOR (PAM) pathway mutation can include a means for identifying one or more PAM pathway mutation comprising one or more nucleic acid primer, nucleic acid primer pair, nucleic acid probe, and/or an antibody specific for said mutation.

As embodied herein, the kit can include techniques for detecting a gain-of-function mutation in the PAM pathway and for detecting mutation in PIK3CA gene. In certain embodiments, the mutation can be at amino acid 4, 364, 1016, 1035, or 1043 of PIK3CA protein, or at equivalent positions of homologous sequences thereto. For the purpose of example and not limitation, the mutation can be selected from the group consisting of R4Q, G364R, F1016C, A1035V, M1043I, and M1043V.

As embodied herein, the one or more primer, primer pair, probe, and/or antibodies within the kit can constitute at least 10 percent of the primers, primer pairs, probes, and antibodies in the kit. In certain embodiments, the kit can include a positive control. In certain embodiments, the kit can further include a pharmaceutical formulation for use in treating M-GBM in a subject in need thereof, comprising at least an effective amount of the PAM pathway inhibiting agent.

### BRIEF DESCRIPTION OF THE DRAWINGS

FIG. 1A is a graph depicting the number of somatic mutations in 114 Patients. FIG. 1B is a graph illustrating the clinic and genetic profile of patients.

FIG. 1C is a graph providing a Pyramids plot highlighting the correlation between different features.

FIG. 1D is a graph providing a 3-D bubble plot illustrating the mutation frequency of somatic non-synonymous mutations in exclusively initial (left axis), exclusively recurrence (right axis), and in common (upper axis).

FIGS. 2A-2D show GISTIC2 qplots of tumors. FIGS. 2A-2B are graphs providing qplots of initial tumors. FIGS. 2C-2D are graphs providing qplots of recurrent tumors.

FIGS. 3A-3F are graphs providing MutComFocal reports of frequently mutated, amplified, and deleted genes in initial and recurrent GBM samples.

FIG. 4A-4B are graphs providing loss of heterozygosity (LOH) analysis based on WES data. FIG. 4A represents LOH regions in initial tumors. FIG. 4B represents LOH regions in recurrent tumors.

FIG. 5 is a graph providing circos plot of MGMT fusions.

FIG. 6 is a graph depicting experimental validation of MGMT fusion in patient R114.

FIG. 7 is a graph showing MGMT expression, methylation, and overall survival in IDH1 wild type primary GBM.

FIG. 8A is a graph showing the fraction of different types of nucleotide changes that are related to Temozolomide (TMZ).

FIG. 8B is a graph depicting HM Score and mutation load.

FIG. 8C is a graph providing silent/missense ratio analysis.

FIG. 8D is a graph providing expression comparison between three gene clusters: HM genes, mutated (M) genes, and non-mutated (NM) genes.

FIG. 9A is a graph providing moduli space of GBM evolution trees.

FIG. 9B is a graph providing a model of branching tumor evolution.

FIG. 9C is a graph showing the relationship between estimated substitution rates before and after treatment, in substitutions per Mb-yr (median and interquartile range for each patient).

FIG. 9D is a graph showing cross-sectional integration of longitudinal data by tumor evolutionary directed graph.

FIG. 10A is a graph illustrating the branching model used in Example 1 of the present disclosure.

FIG. 10B is a graph showing an alternative model in which a lineage descended from the most recent common ancestor (MRCA) of the initial sample is selected by therapy to be a progenitor of the recurrent sample (“selection model”).

FIG. 10C is a graph providing a scatterplot/histogram of mutational patterns that distinguish the two models.

FIG. 10D is a graph providing a correlation between mutation load and age of primary GBM WES-based on TCGA samples.

FIG. 10E is a graph showing the distribution of estimated time before diagnosis when the initial and recurrent lineages diverged, in years (median and 95% credible interval for each patient).

FIG. 11 is a graph showing that no relationship is evident between pre-treatment substitution rate and age at diagnosis (median and 95% credible interval shown for each patient).

FIG. 12 is a graph depicting a volcano plot of fold-change in substitution rate following treatment (median and 95% credible interval of ratio of post- to pre-treatment substitution rates).

FIG. 13 is a graph providing tumor evolutionary directed graph of both gene mutations and copy number alterations.

FIGS. 14A-14E provide evidence of clonal switching of key driver genes during GBM evolution. FIGS. 14A-14B are two graphs showing rearrangement of EGFR exons based on WES data. FIGS. 14C-14E are graphs providing Sanger validation of mutations of some key driver genes.

FIGS. 15A-15D show clonal replacement in key driver genes. FIG. 15A is a table showing that mutations of seven key GBM drivers (EGFR, TP53, PDGFRA, PTEN, ATRX, NF1, and RB1) were replaced by different mutations in the same genes. FIG. 15B is a graph showing PDGFRA mutational replacement in one patient. FIG. 15C is a graph showing TP53 mutational replacement in one patient. FIG. 15D is a graph showing EGFR mutational replacement in one patient.

FIGS. 16A-16C are graphs providing mutation contour plots, illustrating the variant allele fraction (VAF) of somatic SNVs and small INDELs in untreated and recurrent tumors of patient with clonal switching in key driver genes.

FIG. 17A is a graph depicting expression based GBM subtyping.

FIG. 17B is a graph illustrating the association between expression-based subtype switching and genetic/clinic features.

FIG. 17C is a graph providing the stochastic matrix of GBM subtypes.

FIG. 18 is a graph showing that expression-based GBM subtyping predicts GBM overall survival in recurrent tumor.

FIG. 19 is a graph providing Sanger validation of LTBP4 mutations in cohort INCB.

FIG. 20A is a graph showing that LTBP4 mutation was related to its high expression in recurrent GBM.

FIG. 20B is a graph showing survival analysis of LTBP4 expression in IDH1-wild-type primary GBM patients.

FIG. 20C is a graph providing gene set enrichment analysis.

FIG. 20D is an image depicting knocking down of LTBP4 in two cell lines.

FIGS. 20E-20F are two graphs showing gene expression changes in U87 (FIG. 20E) and U251 (FIG. 20F).

FIGS. 20G-20H are two graphs showing cell proliferation of U87 (FIG. 20G) and U251 (FIG. 20H).

FIG. 21 is a graph depicting the calibration of copy number methods.

FIG. 22 is a graph showing the fraction of patients losing initial mutations.

FIG. 23A is a graph showing a schematic representation of glioma genomic heterogeneity and differential drug-response analysis.

FIG. 23B is a graph providing somatic mutations for 83 glioma multiregion or multisector longitudinal specimens from 30 patients.

FIG. 24A is a graph showing landscape of somatic mutations for 22 pair longitudinal cohort.

FIG. 24B is a graph showing shared or private presence of major GBM driver gene alterations in 29 multisector cases from SMC and TCGA cohort.

FIG. 24C is a graph providing disjoint mutation pattern of EGFR in multisector samples.

FIGS. 25A-25D provide comparison of genetic heterogeneity across glioma multisector and longitudinal samples. FIG. 25A is a graph showing Nei's genetic distances for each of the indicated groups. FIG. 25B is a graph illustrating of leave-one-out results from multinomial logistic regression. FIG. 25C is a graph showing tumor evolution behind the Big Bang and multiverse models. FIG. 25D are pie charts depicting the frequencies of PIK3CA mutations in multifocal/multicentric glioblastomas (M-GBMs) and solitary glioblastomas (S-GBMs).

FIGS. 26A-26B show shared clonal and subclonal mutation ratios. FIG. 26A is a graph providing the percentage of clonal events shared among tumor fragments of different groups represented as violin plots. FIG. 26B is a graph providing shared sub-clonal mutation ratio among the groups.

FIG. 27 is a graph providing moduli space to represent sample pairs from the same patient.

FIGS. 28A-28D provide representative MRIs of GBM subgroups. FIG. 28A is an image providing typical T1CE and FLAIR MR images for solitary tumors. FIG. 28B is an image showing solitary with satellite lesions. FIG. 28C is an image showing multicentric tumors. FIG. 28D is an image showing multifocal tumors.

FIGS. 29A-29D provide mutation profiles in multifocal/multicentric (M-) and solitary (S-) GBMs. FIG. 29A is a graph showing mutational profiles (right panel) and proportions (left panel) of representative GBM-associated genes in M- or S-GMBs. FIG. 29B is a graph showing survival analysis of M-GBM. FIG. 29C is a graph showing survival analysis of PIK3CA mutant GBM. FIG. 29D are pie charts showing enrichment of PIK3CA mutations in M-GBM in IDH1 wild type GBM.

FIG. 30A is a graph providing expression profiles of individual tumor cells from three samples of GBM9 according to expression subtype.

FIG. 30B is a graph providing topological representation of the expression data of individual tumor cells from GBM9, labeled by sample of origin.

FIG. 30C is a graph providing topological representations of GBM9 where tumor cells are labeled by expression of EGFR.

FIG. 30D is a graph providing topological representations of GBM9 where tumor cells are labeled by expression of mitotic genes.

FIG. 30E is a graph providing expression profiles of individual tumor cells from GBM10 (two samples: 5-ALA⁺ and 5-ALA⁻).

FIG. 30F is a graph providing topological representations of expression data for individual tumor cells from patients GBM10.

FIG. 30G is a graph showing topological representations of expression data for individual tumor cells from patients GBM2.

FIG. 30H is a graph showing expression profiles of individual tumor cells from patient GBM2 shown according to GBM expression subtype.

FIG. 31 is a graph showing tumor phylogenies for GBM9 (multicentric), GBM10 (5-ALA), and GBM2 (local) with representative preoperative MR images.

FIGS. 32A-32C provide status of somatic driver mutations, copy number alterations, and structure rearrangement for multisector samples from GBM2, 9, and 10. FIG. 32A is a chart showing somatic mutations. FIGS. 32B and 32C are graphs showing experimental validation of ATRX fusion in GBM10.

FIG. 33 is a graph depicting distribution of average graph distances between cells in the topological representation of FIG. 30B.

FIGS. 34A-34B show volcano plots of single cell expression. FIG. 34A is a graph showing in GBM10, single cells from sample with 5-ALA (−) that were compared with those from 5-ALA (+). FIG. 34B is a graph showing in GBM2, single cells from margin sample that were compared with those from main tumor.

FIG. 35A-35E show the topological representation of the expression data of tumor cells from patient GBM10 labelled by the expression level. FIG. 35A is a graph showing MET. FIG. 35B is a graph showing CD44. FIG. 35C is a graph showing CD97. FIG. 35D is a graph showing OLIG1. FIG. 35E is a graph showing PDGFRA. FIGS. 35F-3511 show the topological representation of the expression data of tumor cells from patient GBM2 labelled by the expression level. FIG. 35F is a graph showing CD44. FIG. 35G is a graph showing cell migration genes. FIG. 35H is a graph showing mitotic genes.

FIG. 36A is a graph depicting chemical screening of multiregion PDCs.

FIG. 36B is a graph showing violin plots for the SCC of drug responses for the groups described in FIG. 36A.

FIG. 36C is a graph showing mean values of the AUCs for six PI3K-AKT-mTOR (PAM) inhibitors for PDCs isolated from M-GBMs or S-GBMs.

FIG. 36D is a graph providing the normalized z score for each PDC obtained from the AUCs for the indicated drug classes used in FIG. 36A and FIG. 36B.

FIG. 36E is a graph providing preoperative T1-weighted contrast-enhanced magnetic resonance images (MRIs) and key genomic alterations found in the corresponding tumors and their derivative cells for a patient with multicentric disease (GBM9).

FIG. 36F is a graph showing a scatterplot of the AUCs for 40 cancer-targeting compounds on GBM9 PDCs that were derived from the left- and right-side tumors in the frontal lobes.

FIG. 37 is a graph showing violin plots of AUCs against PAM drugs.

FIG. 38 is a graph showing AUC values for 6 representative EGFR inhibitors from initial or recurrent GBMs. P value was calculated using Wilcoxson Ranksum test.

FIG. 39 is a graph providing median values of AUCs of 6 EGFR inhibitors against PDCs from initial and recurrent tumors of indicated patients.

FIGS. 40A-40D show drug response correlation plots using AUCs for 40 different chemicals of multisector PDCs from indicated cases. FIG. 40A is a graph showing GBM1.

FIG. 40B is a graph showing GBM 17. FIG. 40C is a graph showing GBM19. FIG. 40D is a graph showing GBM20. FIGS. 40A and 40B further include graphs showing phylogenetic trees derived from somatic alterations in accordance with spatial difference with representative MRIs.

FIG. 41A is an image depicting representative MRIs of GBM14 at pre- and post-surgical resection, and drop metastasis at ipsilateral meninges.

FIG. 41B is a graph showing phylogenetic reconstruction using somatic variants. FIG. 41C is a graph showing correlation plot using AUCs for 40 molecular target agents of PDCs from initial and leptomeningeal relapsed tumors.

FIG. 41D is a graph showing representative MR images of GBM15 at pre- and post-surgical resection, and 1st recurrence at distant site.

FIG. 41E is a graph showing immunohistochemical analysis to analyze the activities of EGFR, AKT and mTOR, potential downstream molecules of driver genes including EGFR and PTEN.

FIG. 41F is a graph depicting the evolutionary divergence of GBM15 using longitudinal pair tissues derived from somatic variants.

FIG. 41G is a graph showing correlation plot using AUCs for 40 drugs of PDCs derived from initial or recurrent tumors of GBM15.

FIG. 42A is a graph showing dose response curves of BKM120 (PI3K), selumetinib (MEK) and afatinib (EGFR) demonstrated in both right or left tumor-derived cells of GBM9.

FIG. 42B is a graph showing limiting dilution assay (LDA) of right and left tumor-derived cells.

FIG. 42C is an image showing Western blot analysis to measure the activities of AKT and S6K, key downstreams of EGFR, PI3K and MEK signaling pathways.

FIG. 42D is a graph showing representative MR (T1CE) or perfusion CT images after tumor resection.

FIG. 43 is a graph providing the correlation between the number of cores/sectors per tumor mass and false discovery rate of detecting clonal mutation.

### DETAILED DESCRIPTION

The presently disclosed subject matter relates to methods and compositions for treating multifocal/multicentric glioblastoma (M-GBM) using a PI3K-AKT-mTOR (PAM) pathway inhibiting agent. It was found that there is an enrichment of PIK3CA mutations in M-GBM and that patient derived cells (PDCs) from M-GBMs are more sensitive to PAM pathway inhibitors than PDCs from solitary tumors.

In certain aspects, the present disclosure provides methods for treating a subject with M-GBM including obtaining at least two M-GBM tumor samples from different locations within a subject, extracting genomic DNA from each of theat least two tumor samples to obtain at least two corresponding extracted genomic DNA samples, and determining whether the subject has a PAM pathway mutation in each of the at least two extracted genomic DNA samples. If a PAM pathway mutation is determined in each of the at least two DNA samples, the method can further include treating the subject with an effective amount of a PAM pathway inhibiting agent.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the presently disclosed subject matter. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of”, and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, “treatment” or “treating” refers to inhibiting the progression of a disease or disorder, or delaying the onset of a disease or disorder, whether physically, e.g., stabilization of a discernible symptom, physiologically, e.g., stabilization of a physical parameter, or both. As used herein, the terms “treatment,” “treating,” and the like, refer to obtaining a desired pharmacologic and/or physiologic effect. The effect can be prophylactic in terms of completely or partially preventing a disease or condition, or a symptom thereof and/or can be therapeutic in terms of a partial or complete cure for a disease or disorder and/or adverse effect attributable to the disease or disorder. “Treatment,” as used herein, covers any treatment of a disease or disorder in an animal or mammal, such as a human, and includes: decreasing the risk of death due to the disease; preventing the disease of disorder from occurring in a subject which can be predisposed to the disease but has not yet been diagnosed as having it; inhibiting the disease or disorder, i.e., arresting its development (e.g., reducing the rate of disease progression); and relieving the disease, i.e., causing regression of the disease.

As used herein, the term “subject” includes any human or nonhuman animal. The term “nonhuman animal” includes, but is not limited to, all vertebrates, e.g., mammals and non-mammals, such as nonhuman primates, dogs, cats, sheep, horses, cows, chickens, amphibians, reptiles, etc. In certain embodiments, the subject is a pediatric patient. In certain embodiments, the subject is an adult patient.

As used herein, an “effective amount” refers to an amount of the compound sufficient to treat, prevent, or manage the disease. An effective amount can refer to the amount of a compound that provides a therapeutic benefit in the treatment or management of the disease, and as such, an “effective amount” depends upon the context in which it is being applied. In the context of administering a composition to treat and/or to reduce the severity of M-GBM in a subject, an effective amount of a composition described herein is an amount sufficient to treat and/or ameliorate M-GBM cancer cell growth, as well as decrease the severity and/or reduce the likelihood of M-GBM cancer cell growth. In the context of administering a composition to treat and/or to reduce the severity of recurrent GBM in a subject, an effective amount of a composition described herein is an amount sufficient to treat and/or ameliorate recurrent GBM cancer cell growth, as well as decrease the severity and/or reduce the likelihood of recurrent GBM cancer cell growth. The decrease can be a 10%, 20%, 30%, 40%, 50%, 60%, 70%, 80%, 90%, 95%, 98%, or 99% decrease in severity of cancer cell growth, or likelihood of developing cancer. An effective amount can be administered in one or more administrations. Further, a therapeutically effective amount with respect to a PAM pathway inhibitor or TGF-β pathway inhibitor of the disclosure can mean the amount of therapeutic alone, or in combination with other therapies, that provides a therapeutic benefit in the treatment or management of the disease, which can include a decrease in severity of disease symptoms, an increase in frequency and duration of disease symptom-free periods, or a prevention of impairment or disability due to the disease affliction. The term can encompass an amount that improves overall therapy, reduces or avoids unwanted effects, or enhances the therapeutic efficacy of or synergies with another therapeutic agent. Animal models accepted in the art as models of disease (e.g., M-GBM or recurrent GBM) can be used to test particular compounds, routes of administration etc., to determine appropriate amounts of therapeutic treatments of the disclosure.

As embodied herein, at least two M-GBM tumor samples can be obtained from different locations within a subject. For example, the different locations can each correspond to different M-GBM tumors within the body of the subject. Alternately or additionally, the different locations can each correspond to different regions of a single M-GBM tumor. In certain embodiments, two or more samples can be obtained from a single tumors and at least one additional sample can be obtained from a different tumor.

The M-GBM tumor samples or reccurent GBM tumor samples of the present disclosure can be obtained using any suitable techniques, as known in the art. For example, the method can include obtaining a biopsy of the subject from the one or more locations. As embodied herein, and without limitation, the samples can be obtained using invasive or minimally invasive techniques, including but not limited to open surgery or needle biopsy.

After the two or more M-GBM tumor or recurrent GBM samples have been obtained, the method can further include extracting genomic DNA from each of the tumor samples. As used herein, the term “genomic DNA” refers to chromosomal DNA that encodes the genome of an organism. In certain embodiments, genomic DNA is isolated from a human cell. In certain embodiments, genomic DNA is isolated from an animal cell. Genomic DNA can be extracted using any suitable technique, including chemical and physical techniques, as known in the art. The genomic DNA can be sequenced to determine whether DNA from any or all of the tumor samples contains a mutation in PI3K-AKT-mTOR (PAM) pathway.

The phosphoinositide 3-kinase (PI3K)-AKT-mammalian target of rapamycin (mTOR) pathway is a vital signaling pathway involved in cellular proliferation, survival, metabolism and motility. PI3K is activated upstream by the binding of a growth factor or ligand to its cognate growth factor receptor tyrosine kinases. PI3K activation leads to the synthesis of the second messenger phosphatidylinositol-(3,4,5)-triphosphate (PIP₃) at the plasma membrane, which in turn results in phosphorylation of AKT, a serine/threonine kinase. Phosphorylation of AKT stimulates protein synthesis and cell growth by activating mTOR via effects on the intermediary tuberous sclerosis 1/2 complex (TSC1/2).

Class IA PI3Ks are heterodimeric proteins made up of a p110 catalytic subunit and a p85 regulatory subunit, and are involved in carcinogenesis. PIK3CA encodes for the alpha isoform of PI3K, p110α, the catalytic subunit of the PI3K holoenzyme. PIK3CA is frequently mutated or amplified in solid tumors.

There are three isoforms of AKT: AKT1, AKT2, and AKT3. They share a conserved domain structure which includes an N-terminal pleckstrin homology domain, a kinase domain, and a C-terminal regulatory domain. AKT1 is expressed in all tissues, while AKT2 is mostly expressed in insulin-responsive tissues and AKT3 is highly expressed in brain and testes.

mTOR is an atypical serine/threonine kinase that is present in two distinct complexes, mTOR complex 1 (mTORC1) and mTOR complex 2 (mTORC2). mTORC1 is the target of rapamycin and rapamycin analogues and is involved in promoting cellular growth and limiting catabolic processes such as autophagy. mTORC2 regulates cellular survival by activing AKT and cytoskeletal dynamics, as well as controlling ion transport.

Mutations in the PAM pathway related to the presently disclosed subject matter include, but are not limited to, gain-of-function mutations that activate the PAM pathway. In certain embodiments, the mutation in the PAM pathway is in PIK3CA gene. In certain embodiments, the mutation in the PAM pathway is in one or more of AKT1, AKT2, AKT3, and/or mTOR genes. These loci can have single or multiple mutations that can be substitutions, insertions, deletions, copy number alterations, and/or gene fusions.

In certain non-limiting embodiments, the PIK3CA is a human PIK3CA protein having an amino acid sequence as set forth by GenBank Accession No. NP_006209 (SEQ ID NO: 1), or a protein having a sequence that is at least 80 percent, at least 85 percent, at least 90 percent, at least 95 percent, or at least 99 percent homologous thereto (homology, as that term is used herein, can be measured using standard software such as BLAST or FASTA). The PIK3CA is encoded by a nuclei acid as set forth by GenBank Accession No. NM_006218 (SEQ ID NO: 2), or a nucleic acid having a sequence that is at least 80 percent, at least 85 percent, at least 90 percent, at least 95 percent, or at least 99 percent homologous thereto.

In certain non-limiting embodiments, the mutation of PIK3CA is at amino acid 4, 364, 1016, 1035, or 1043 of PIK3CA protein, or at equivalent positions of homologous sequences thereto. In certain non-limiting embodiments, the mutation of PIK3CA is selected from the group consisting of R4Q, G364R, F1016C, A1035V, M1043I, and M1043V. As used herein, the term “homologous sequences” refers to sequences that share a significant sequence similarity as determined by an alignment of the sequences. For example, two sequences can be about 50%, 60%, 70%, 80%, 90%, 95%, 99%, or 99.9% homologous. The alignment is carried out by algorithms and computer programs including, but not limited to, BLAST, FASTA, and HMME, which compares sequences and calculates the statistical significance of matches based on factors such as sequence length, sequence identify and similarity, and the presence and length of sequence mismatches and gaps. Homologous sequences can refer to both nucleic acid and protein sequences.

As embodied herein, if a mutation in PAM pathway is present in at least two DNA samples, the method can further include treating the subject with an effective amount of a PAM pathway inhibiting agent. For example, the method can include treating the subject with an effective amount of an agent that inhibits the PAM pathway if genomic DNA from two or more, three or more, four or more, or five or more M-GBM tumor samples contain a mutation in PAM pathway. Thus, the presently disclosed subject matter provides for agents that inhibit the PAM pathway. In certain embodiments, the agent inhibits the ability of PI3K, AKT, and/or mTOR to phosphorylate a target protein. In certain embodiments, the agent decreases the expression level or activity of PI3K, AKT, and/or mTOR.

In certain embodiments, the PAM pathway inhibiting agent is a pan-PI3K inhibitor. In certain embodiments, the PAM pathway inhibiting agent is an isoform specific PI3K inhibitor. In certain embodiments, the agent is a dual PI3K-mTOR inhibitor. In certain embodiments, the PAM pathway inhibiting agent is an AKT inhibitor. In certain embodiments, the PAM pathway inhibiting agent is an mTORC1 inhibitor. In certain embodiments, the agent is a dual mTORC1-mTORC2 inhibitor.

In certain embodiments, the PAM pathway inhibiting agent is selected from the group consisting of BKM120 (Buparlisib), XL147 (Pilaralisib), GDC0941 (Pictilisib), BYL719 (Alpelisib), GDC0032 (Tazelisib), NVP-BEZ235, LY3023414, GSK2126458, BEZ235, PF-05212384 (PKI-587), AZD5363, MK-2206, GSK21411795 (Uprosertib), GDC-0068 (Lpatasertib), LNK128, AZD2014, AZD8055, MLN0138, CC-223, RAD001 (Everolimus), rapamycin (Sirolimus), CCI-779 (Temsirolimus), AP23573 (Ridaforolimus), and combinations thereof.

In certain embodiments, the PAM pathway inhibiting agent includes a nucleic acid that specifically binds to a nucleic acid encoding PIK3CA, and reduces PI3K expression and/or activity. In certain embodiments, the PAM pathway inhibiting agent includes a nucleic acid that specifically binds to a nucleic acid encoding AKT1, AKT2, or AKT3, and reduces AKT expression and/or activity. In certain embodiments, the PAM pathway inhibiting agent includes a nucleic acid that specifically binds to a nucleic acid encoding mTOR, and reduces mTOR expression and/or activity. In certain embodiments, the PAM pathway inhibiting agent includes a microRNA (miRNA) molecule, small interfering RNA (siRNA) molecule, short hairpin RNA (shRNA) molecule, catalytic RNA molecule, and/or catalytic DNA molecule.

As embodied herein, the PAM inhibitor or TGF-β pathway inhibitor of the present disclosure can be formulated into compositions suitable for pharmaceutical administration. Pharmaceutical compositions of this disclosure also can be administered in combination therapy, i.e., combined with other agents. In certain embodiments, the combination therapy can include a PAM inhibitor or TGF-β pathway inhibitor of the present disclosure combined with at least one additional agent. In certain embodiments, the additional agent is an additional therapeutic agent, a stabilizing compound, and/or a biocompatible pharmaceutical carrier.

As used herein the term “pharmaceutically acceptable carrier” is a pharmaceutically acceptable solvent, suspending agent or vehicle for delivering the PAM inhibitor to the animal or human. The carrier can be liquid or solid and is selected with the planned manner of administration being used. For example, a pharmaceutically acceptable carrier is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated.

Pharmaceutically acceptable carriers are generally nontoxic to recipients at the dosages and concentrations employed, and include, but are not limited to, buffers such as phosphate, citrate, and other organic acids; antioxidants including ascorbic acid and methionine; preservatives (such as, but not limited to, octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol and m-cresol); low molecular weight (less than about 10 residues) polypeptides; proteins, such as serum albumin, gelatin or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). In certain embodiments, a suitable pharmaceutically acceptable carrier can include one or more of water, saline, phosphate buffered saline, dextrose, glycerol, ethanol or combinations thereof.

A pharmaceutical composition of the disclosure is formulated to be compatible with its intended route of administration. For example, solutions or suspensions used for parenteral, intradermal, or subcutaneous application can include one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (such as water) or dispersions and sterile powders for the extemporaneous preparation of sterile injectable solutions or dispersion. For intravenous administration, suitable carriers can include physiological saline, bacteriostatic water, Cremophor EL. (BASF, Parsippany, N.J.) or phosphate buffered saline (PBS). In certain embodiments, the composition must be sterile and can be fluid to the extent that easy syringability exists. In certain embodiments, the composition must be stable under the conditions of manufacture and storage and must be preserved against the contaminating action of microorganisms such as bacteria and fungi. In certain embodiments, the carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In certain embodiments, isotonic agents, for example, sugars, polyalcohols such as manitol and sorbitol, and sodium chloride can be included in the composition. Prolonged absorption of the injectable compositions can be brought about by including in the composition an agent that delays absorption, for example, aluminum monostearate or gelatin.

In certain embodiments, sterile injectable solutions can be prepared by incorporating the active compound (i.e., the PAM inhibitor) in the required amount in an appropriate solvent with one or a combination of ingredients enumerated above, as required, followed by filtered sterilization. Dispersions can be prepared by incorporating the active compound into a sterile vehicle which contains a basic dispersion medium and the required other ingredients from those enumerated above. In the case of sterile powders for the preparation of sterile injectable solutions, methods of preparation can include vacuum drying and freeze-drying, which yields a powder of the active ingredient plus any additional desired ingredient from a previously sterile-filtered solution thereof.

In certain embodiments, oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In certain embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

In certain embodiments, liposomal suspensions (including liposomes targeted to tumor cells with monoclonal antibodies to tumor antigens) can also be used as pharmaceutically acceptable carriers. These can be prepared according to methods known to those skilled in the art, for example, as described in U.S. Pat. No. 4,522,811, and International Patent Application Serial No. PCT/US94/07327. For example, liposome formulations can be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of invariant chain protein or peptide is then introduced into the container. The container is then swirled by hand to free lipid material from the sides of the container and to disperse lipid aggregates, thereby forming the liposomal suspension.

Pharmaceutical compositions, including, but not limited to, such liposomal suspensions and other microencapsulated compositions, can be combined with targeting agents to allow for tissue specific delivery of the PAM inhibitor of the disclosure. In certain embodiments, such targeting can be achieved, without limitation, through the use of tissue specific antibodies and antibody mimetics. Non-limiting examples of antibody mimetics include, but are not limited to, molecules such as Affibodies, DARPins, Anticalins, Avimers, and Versabodies, all of which employ binding structures that, while they mimic traditional antibody binding and therefore can be used to target peptides to tissues specifically expressing the antigen recognized by the mimetic, are generated from and function via distinct mechanisms.

Pharmaceutical compositions can also be prepared wherein the PAM inhibitor or TGF-β pathway inhibitor of the disclosure is covalently or non-covalently attached to a nanoparticle. By way of example, but not limitation, a nanoparticle can be a dendrimer, such as the polyamidoamine employed in Kukowska-Latallo et al., (2005) Cancer Res., vol. 65, pp. 5317-24, which is incorporated herein by reference in its entirety. Other dendrimers that can be used in conjunction with the PAM inhibitor of the instant disclosure include, but are not limited to, polypropylenimine dendrimers as described in U.S. Pat. No. 7,078,461, which is hereby incorporated by reference in its entirety.

In certain embodiments, the oral or parenteral compositions can be formulated in dosage unit form for ease of administration and uniformity of dosage. “Dosage unit form” as used herein refers to physically discrete units suited as unitary dosages for the subject to be treated, each unit containing a predetermined quantity of active compound calculated to produce the desired therapeutic effect in association with the required pharmaceutical carrier. The specification for the dosage unit forms of the present disclosure are dictated by and directly dependent on (a) the unique characteristics of the active compound and the particular therapeutic effect to be achieved, and (b) the limitations inherent in the art of compounding such an active compound for the treatment of individuals.

Actual dosage levels of the active ingredients in the pharmaceutical compositions of the present disclosure can be varied so as to obtain an amount of the active ingredient that is effective to achieve the desired therapeutic response for a particular patient, composition, and mode of administration, without being toxic to the subject. The selected dosage level will depend upon a variety of pharmacokinetic factors including the activity of the particular compositions of the present disclosure employed, or the ester, salt or amide thereof, the route of administration, the time of administration, the rate of excretion of the particular compound being employed, the duration of the treatment, other drugs, compounds, and/or materials used in combination with the particular compositions employed, the age, sex, weight, condition, general health, and prior medical history of the patient being treated, and like factors as well known in the medical arts.

Single or multiple administrations of formulations can be given depending on the dosage and frequency as required and tolerated by the patient. In certain embodiments, the formulations should provide a sufficient quantity of active agent to effectively treat, prevent or ameliorate the disease to be treated, e.g., M-GBM, recurrent GBM, or symptoms or complications thereof as described herein.

In certain embodiments, a composition of the present disclosure can be administered via one or more routes of administration using one or more of a variety of methods known in the art. As will be appreciated by the skilled artisan, the route and/or mode of administration will vary depending upon the desired results. Routes of administration for the PAM inhibitor of this disclosure include, but are not limited to, intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In certain embodiments, a PAM inhibitor or a TGF-β pathway inhibitor of this disclosure can be administered via a non-parenteral route, such as a topical, epidermal or mucosal route of administration, for example, intranasally, orally, vaginally, rectally, sublingually or topically.

In certain embodiments, therapeutic compositions can be administered with medical devices known in the art. For example, in certain embodiments, a therapeutic composition of this disclosure can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

As described above, in certain non-limiting embodiments, the present disclosure provides for a method for treating a subject with multifocal/multicentric glioblastoma (M-GBM) including: obtaining at least two M-GBM tumor samples from different locations within a subject, extracting genomic DNA from each of theat least two tumor samples to obtain at least two corresponding extracted genomic DNA samples, and determining whether the subject has a PAM pathway mutation in each of the at least two extracted genomic DNA samples. If a PAM pathway mutation is determined in each of the at least two DNA samples, the method can further include treating the subject with an effective amount of a PAM pathway inhibiting agent.

In certain embodiments, the subject has a gain-of-function mutation that activates the PAM pathway in at least two M-GBM tumor samples from different locations. In certain embodiments, the subject has a mutation in PIK3CA gene in at least two M-GBM tumor samples from different locations. In certain embodiments, the subject has a mutation at amino acid 4, 364, 1016, 1035, or 1043 of PIK3CA protein, or at equivalent positions of homologous sequences thereto in at least two M-GBM tumor samples from different locations. In certain embodiments, the subject has a mutation selected from the group consisting of R4Q, G364R, F1016C, A1035V, M1043I, and M1043V of PIK3CA protein in at least two M-GBM tumor samples from different locations. In certain embodiments, the subject has a mutation in one or more of AKT1, AKT2, AKT3, and/or mTOR genes in at least two M-GBM tumor samples from different locations. In certain non-limiting embodiments, the present disclosure provides for further treating the subject with an amount of a second inhibitor of the PAM pathway in an amount that, together with the first inhibitor, effectively treats the M-GBM.

The presence of a mutation in a component of the PAM pathway may be determined using methods known in the art. For example, but not by way of limitation, the presence of a mutation selected from the group consisting of R4Q, G364R, F1016C, A1035V, M1043I, and M1043V of PIK3CA protein may be determined using methods such as PCR amplification, nucleic acid probe hybridization, and/or using antibodies specific for a protein or peptide bearing the mutation.

In certain embodiments, the present disclosure provides for a method for treating M-GBM in a subject include administering, to the subject, an effective amount of a PAM pathway inhibiting agent. In certain embodiments, the method can further include administering to the subject an additional therapeutic agent, a stabilizing compound, and/or a biocompatible pharmaceutical carrier.

In certain embodiments, the agent is administered in an amount effective to increase cell death of GBM tumor cells in a treated subject, lengthen subject survival, or a combination thereof. In certain embodiments, an effective amount of an agent described herein is an amount which treats or reduces the severity of M-GBM in a subject. For example, treating or reducing the severity of M-GBM refers to an amelioration in the clinical symptoms or signs of cancer, for example, but not by way of limitation, reduction in tumor volume, and/or reduction in cells expressing cancer markers such as, for example but not limited to, HER2, EGFR, PCNA, or other proliferative markers known in the art. In certain embodiments, the effective amount of the agent is an amount that increases the number of apoptotic cancer cells in the subject, for example, as evidenced by an increase in cleaved caspase 3 and/or 7, cleaved PARP, and/or TUNEL.

In certain embodiments, an agent of the present disclosure, e.g., an inhibitor of the PAM pathway or the TGF-β pathway can be administered to a subject at an amount of about 0.01 mg/kg to about 10 mg/kg. For example, and not by way of limitation, an inhibitor can be administered at an amount of about 0.01 mg/kg to about 9.5 mg/kg, about 0.01 mg/kg to about 9 mg/kg, about 0.01 mg/kg to about 8.5 mg/kg, about 0.01 mg/kg to about 8 mg/kg, about 0.01 mg/kg to about 7.5 mg/kg, about 0.01 mg/kg to about 7 mg/kg, about 0.01 mg/kg to about 6.5 mg/kg, about 0.01 mg/kg to about 6 mg/kg, about 0.01 mg/kg to about 5.5 mg/kg, about 0.01 mg/kg to about 5 mg/kg, about 0.01 mg/kg to about 4.5 mg/kg, about 0.01 mg/kg to about 4 mg/kg, about 0.01 mg/kg to about 3.5 mg/kg, about 0.01 mg/kg to about 3 mg/kg, about 0.01 mg/kg to about 2.5 mg/kg, about 0.01 mg/kg to about 2 mg/kg, about 0.01 mg/kg to about 1.5 mg/kg, about 0.01 mg/kg to about 1 mg/kg, about 0.01 mg/kg to about 0.5 mg/kg, about 0.01 mg/kg to about 0.1 mg/kg, about 0.1 mg/kg to about 10 mg/kg, about 0.5 mg/kg to about 10 mg/kg, about 1 mg/kg to about 10 mg/kg, about 1.5 mg/kg to about 10 mg/kg, about 2 mg/kg to about 10 mg/kg, about 2.5 mg/kg to about 10 mg/kg, about 3 mg/kg to about 10 mg/kg, about 3.5 mg/kg to about 10 mg/kg, about 4 mg/kg to about 10 mg/kg, about 4.5 mg/kg to about 10 mg/kg, about 5 mg/kg to about 10 mg/kg, about 5.5 mg/kg to about 10 mg/kg, about 6 mg/kg to about 10 mg/kg, about 6.5 mg/kg to about 10 mg/kg, about 7 mg/kg to about 10 mg/kg, about 7.5 mg/kg to about 10 mg/kg, about 8 mg/kg to about 10 mg/kg, about 8.5 mg/kg to about 10 mg/kg, about 9 mg/kg to about 10 mg/kg, or about 9.5 mg/kg to about 10 mg/kg, e.g., by one or more separate administrations, or by continuous infusion. In certain embodiments, an inhibitor of the present disclosure can be administered at an amount of about 0.5 mg/kg to about 5 mg/kg, or about 1 mg/kg to about 3 mg/kg, e.g., about 2 mg/kg.

The treatment methods of the present disclosure can be administered alone or in conjunction with another form of pharmaceutical and/or surgical therapy. Non-limiting examples of pharmaceutical treatments and/or agents include, but are not limited to, treatment with one or more of: an anti-angiogenic agent, a steroid, a beta-blocker, and/or an agent that reduces blood pressure. In certain embodiments, “in conjunction with”, means that an inhibitor of the PAM pathway and another pharmaceutical agent are administered to a subject as part of a treatment regimen or plan. In certain embodiments, being used in conjunction does not require that the PAM pathway inhibitor and the pharmaceutical agent are physically combined prior to administration or that they be administered over the same time frame.

The presently disclosed subject matter further provides kits for determining the presence of a PAM pathway mutation and/or TGF-β pathway mutation.

In certain embodiments, the kit of the present disclosure includes a means for identifying one or more PAM pathway and/or TGF-β pathway mutations as set forth above, including for example, one or more nucleic acid primer, nucleic acid primer pair, nucleic acid probe, or an antibody specific, for said mutation. In particular non-limiting embodiments, the one or more primer, primer pair, probe, or antibodies for identifying relevant pathway member mutations set forth herein constitute at least 10 percent, or at least 30 percent, or at least 50 percent, or at least 75 percent, or all of the primers, primer pairs, probes, or antibodies in the kit. In particular non-limiting embodiments, primers, primer pairs, probes or antibodies are provided for identifying species of relevant mutant pathway members, where said species constitute at least 10 percent, or at least 30 percent, or at least 50 percent, or at least 75 percent, or all species identifiable by the kit.

In non-limiting embodiments, said kit further comprises a positive or negative control for one or more of the mutations represented. As one non-limiting example, the kit may comprise, as positive control(s), one or more sample of nucleic acid corresponding to the R4Q, G364R, F1016C, A1035V, M1043I, and M1043V mutations. In other non-limiting embodiments, the kit may comprise a sample of glioblastoma cells containing one or more relevant mutations. For example, in certain embodiments, the kit can include a positive control of DNA including a PAM pathway mutation, for example and not limitation, a mutation in PIK3CA gene. Additionally or alternatively, the kit can include a negative control of DNA with a normal PAM pathway, for example and not limitation, the wild-type PIK3CA gene. For further example and not limitation, the kit can further include nucleic acid primers for PCR analysis or nucleic acid probes for in situ hybridization analysis to detect the presence of one or more PAM pathway mutations in the extracted genomic DNA samples. As embodied herein the nucleic acid primers and/or nucleic acid probes can be specific to the PAM pathway mutation, e.g., a mutation in PIK3CA gene.

In certain embodiments, the kit of the present disclosure includes a pharmaceutical formulation for use in treating M-GBM in a subject in need thereof, including an effective amount of an agent that inhibits the PAM pathway. In certain embodiments, the kit of the present disclosure includes an agent that inhibits the PAM pathway such as, but not limited to, BKM120 (Buparlisib), XL147 (Pilaralisib), GDC0941 (Pictilisib), BYL719 (Alpelisib), GDC0032 (Tazelisib), NVP-BEZ235, LY3023414, GSK2126458, BEZ235, PF-05212384 (PKI-587), AZD5363, MK-2206, GSK21411795 (Uprosertib), GDC-0068 (Lpatasertib), LNK128, AZD2014, AZD8055, MLN0138, CC-223, RAD001 (Everolimus), rapamycin (Sirolimus), CCI-779 (Temsirolimus), AP23573 (Ridaforolimus), and combinations thereof.

In certain non-limiting embodiments, the kit of the present disclosure includes a means of detecting a mutation of the PAM pathway in multiple M-GBM tumor samples in a subject with M-GBM and instructions regarding treating M-GBM in the subject using an effective amount of an agent that inhibits the PAM pathway. In certain embodiments, the kit includes a means of detecting a gain-of-function mutation that activates the PAM pathway. In certain embodiments, the kit includes a means of detecting a mutation in PIK3CA gene. In certain embodiments, the kit includes a means of detecting a mutation at amino acid 4, 364, 1016, 1035, or 1043 of PIK3CA protein, or at equivalent positions of homologous sequences thereto. In certain embodiments, the kit includes a means of detecting a PIK3CA mutation selected from the group consisting of R4Q, G364R, F1016C, A1035V, M1043I, and M1043V. In certain embodiments, the kit includes a means of detecting a mutation in AKT1, AKT2, AKT3, and/or mTOR gene.

In certain embodiments, said means of detecting a mutation can include, for example but not by way of limitation, one or more primer or primer pair for amplification of nucleic acid and subsequent detection of a mutation described above, as embodied in nucleic acid of a subject; one or more nucleic acid probe for detection of a mutation described above, as embodied in nucleic acid of a subject; and/or an antibody, antibody fragment, or single-chain antibody for detection of a protein with mutation described above.

As embodied herein, the presently disclosed techniques can provide for the identification of genetic markers for prognoses of GBM patients and the identification of target therapeutic pathways for GBM. The disclosed subject matter further provides methods for modulating said therapeutic pathways.

In certain aspects, the present disclosure provides methods for the identification of genetic markers corresponding to improved or reduced prognoses of GBM patients. In certain embodiments, the genetic markers can be present in one or both of an initial tumor sample and a recurrent tumor sample. For example, the initial tumor sample can be obtained from a tumor when the patient is diagnosed with GBM and/or prior to treatment of GBM. The recurrent tumor sample can be obtained from a tumor when GBM recurs in the patient. Methods can include obtaining genomic DNA samples from the initial tumor sample and recurrent tumor sample, e.g., using the techniques described above. Methods can further include identifying the presence of genetic markers, if any, in one or both of the tumor samples. For example, the method can include determining the prognosis of a glioblastoma patient based on the presence or absence of one or more genetic markers.

In certain embodiments, the genetic marker can be a mutation in the initial tumor sample or recurrent tumor sample. Information regarding the locations and amounts of mutation(s) exclusive to the initial tumor sample or recurrent tumor sample can be used to generate an evolutionary tree representing clonal evolution between the initial tumor sample and recurrent tumor sample. These methods can be used to identify mutations that occurred prior to diagnosis, or alternatively, during or after treatment of the initial tumor. In this manner, mutations that are more prevalent in patients with recurring GBM can be identified.

Additionally or alternatively, the method can include determining the prognosis of a glioblastoma patient based on the presence or absence of one or more genetic markers corresponding to recurring glioblastoma. In certain embodiments, the genetic marker can be a gene fusion. For example, the gene fusion can involve MGMT. Such gene fusions include, but are not limited to, NFYC-MGMT, BTRC-MGMT, and SAR1A-MGMT. For example, identifying the presence of a gene fusion involving MGMT can result in the determination of a reduced prognosis. The method can also include using the presence or absence of other genetic markers, e.g., mutations, for the determination of an improved or reduced prognosis. In certain embodiments, the genetic marker can be a mutation in LTBP4 gene. Mutations in the LTBP4 gene can lead to increased expression of the gene, causing increased activation of the TGF-β pathway. Activation of the TGF-β pathway can cause increased proliferation of glioma cells and/or aggressive GBM.

In certain aspects, the present disclosure provides methods of identifying a target therapeutic pathway. For example, the target therapeutic pathway can be a pathway that, when activated, results in increased proliferation of glioma cells and/or aggressive glioblastoma. In certain embodiments, the target therapeutic pathway can be the TGF-β pathway. The method can further include identifying the presence or absence of a genetic marker that regulates or is otherwise involved in the target therapeutic pathway. For example, mutations to a gene that is implicated in the regulation of the pathway can indicate increased activation or deactivation of said pathway. In this manner, the method can include using the presence or absence of mutations to such genes for the determination of an improved or reduced prognosis.

In certain aspects, the present disclosure provides methods of modulating a target therapeutic pathway. Methods of modulating a target therapeutic pathway can include determining one or more genetic markers that regulate the target therapeutic pathway, e.g., using the methods described previously, and silencing the one or more genetic markers. For example, the genetic marker(s) can be silenced using RNA interference. In certain embodiments, modulating the target therapeutic pathway can result in decreased proliferation of glioma cells.

The presently disclosed subject matter relates to methods and compositions for treating recurrent GBM using a TGF-β pathway inhibiting agent. In certain aspects, the method of treating recurrent GBM in a subject includes administering, to the subject, an effective amount of a TGF-β pathway inhibiting agent. In certain aspects, the method for treating a subject with recurrent GBM includes obtaining an initial and a recurrent GBM tumor sample from a patient, extracting genomic DNA from the initial and the recurrent tumor sample to obtain at least two corresponding extracted genomic DNA samples each comprising a LTBP4 gene, determining whether the subject has a mutation in the LTBP4 gene in each of the at least two extracted genomic DNA samples, and if a LTBP4 gene mutation is determined in the extracted genomic DNA sample from the recurrent tumor sample, but not in the extracted genomic DNA sample from the initial tumor sample, treating the subject with an effective amount of a TGF-β pathway inhibiting agent.

In certain non-limiting embodiments, the LTBP4 is a human PIK3CA protein having an amino acid sequence as set forth by GenBank Accession No. Q8N2S1 (SEQ ID NO: 3), or a protein having a sequence that is at least 80 percent, at least 85 percent, at least 90 percent, at least 95 percent, or at least 99 percent homologous thereto. The LTBP4 is encoded by a nuclei acid as set forth by GenBank Accession No. NM_001042544 (SEQ ID NO: 4), or a nucleic acid having a sequence that is at least 80 percent, at least 85 percent, at least 90 percent, at least 95 percent, or at least 99 percent homologous thereto.

Mutations in the LTBP4 gene related to the presently disclosed subject matter include, but are not limited to, mutations that increase LTBP4 protein expression. In certain non-limiting embodiments, the mutation of LTPB4 is at amino acid 35, 98, 292, 446, 503, 552, 712, 1060, 1215, 1232, 1236, 1292, 1315, 1325, 1384, or 1416 of LTBP4 protein, or at equivalent positions of homologous sequences thereto. In certain non-limiting embodiments, the mutation of LTBP4 is selected from the group consisting of S35N, S98N, E292K, L446, G503D, N552S, D712N, G1060S, G1215D, Q1232, P1236S, L1292Q, T1315I, P1325L, G1384D, or G1416S.

In certain embodiments, the TGF-β pathway inhibiting agent is selected from the group consisting of CAT-152 (Lerdelimumab), Metelimumab, GC1008 (Fresolimumab), LY2382770, AP12009 (Trabedersen), Belagenpumatucel-L (Lucanix), rhGMCSF/shRNAfurin, P144 (Disitertide), LY2157299 (Galunisertib), TEW-7197, PF-03446962, LY3022859 (IMC-TR1), and combinations thereof.

In certain embodiments, the TGF-β pathway inhibiting agent includes a nucleic acid that specifically binds to a nucleic acid encoding LTBP4, and reduces LTBP4 expression and/or activity. In certain embodiments, the TGF-β pathway inhibiting agent includes a microRNA (miRNA) molecule, small interfering RNA (siRNA) molecule, short hairpin RNA (shRNA) molecule, catalytic RNA molecule, and/or catalytic DNA molecule.

The presently disclosed subject matter further provides kits for treating a subject with reccurent GBM. In certain embodiments, the kit of the present disclosure includes sample containers for accepting an initial and a recurrent GBM tumor samples from a subject; sample analysis components for extracting genomic DNA from each of the two tumor samples to obtain at least two corresponding extracted genomic DNA samples eaching comprising a LTBP4 gene, and for determining whether the subject has a mutation in the LTBP4 gene in each of the at least two extracted genomic DNA samples; instructions for treating recurrent GBM in the subject using an effective amount of a TGF-β pathway inhibiting agent; and a pharmaceutical formulation for use in treating recurrent GBM in a subject in need thereof, including an effective amount of the TGF-β pathway inhibiting agent.

In certain embodiments, the kit includes a means of detecting a LTBP4 mutation that increase LTBP4 protein expression. In certain embodiments, the kit includes a means of detecting a mutation at amino acid 35, 98, 292, 446, 503, 552, 712, 1060, 1215, 1232, 1236, 1292, 1315, 1325, 1384, or 1416 of LTBP4 protein, or at equivalent positions of homologous sequences thereto. In certain embodiments, the kit includes a means of detecting a LTBP4 mutation selected from the group consisting of S35N, S98N, E292K, L446, G503D, N552S, D712N, G1060S, G1215D, Q1232, P1236S, L1292Q, T1315I, P1325L, G1384D, or G1416S.

As embodied herein, the kit can further include a positive control and/or a negative control. For example and not limitation, the kit can include a positive control of DNA including a mutation in LTBP4 gene. Additionally or alternatively, the kit can include a negative control of DNA, for example and not limitation, the wild-type LTBP4 gene. For further example and not limitation, the kit can further include nucleic acid primers for PCR analysis or nucleic acid probes for in situ hybridization analysis to detect the presence of one or more mutations in the LTBP4 gene in the extracted genomic DNA samples. As embodied herein the nucleic acid primers and/or nucleic acid probes can be specific to a mutation in the LTBP4 gene.

In certain embodiments, the kit of the present disclosure includes an agent that inhibits the TGF-β pathway such as, but not limited to, CAT-152 (Lerdelimumab), Metelimumab, GC1008 (Fresolimumab), LY2382770, AP12009 (Trabedersen), Belagenpumatucel-L (Lucanix), rhGMCSF/shRNAfurin, P144 (Disitertide), LY2157299 (Galunisertib), TEW-7197, PF-03446962, LY3022859 (IMC-TR1), and combinations thereof.

### EXAMPLES

The present disclosure is further illustrated by the following Examples which should not be construed as further limiting.

### Example 1: Clonal Evolution of Glioblastoma Under Therapy

This Example provides methods of determining a patients prognosis based on the presence or absence of genetic markers for Glioblastoma (GBM). GBM is a common and aggressive primary brain tumor. To better understand how GBM evolves longitudinal genomic and transcriptomic data of 114 patients was analyzed. The analysis reveals a highly branched evolutionary pattern in which 63% of patients experience expression-based subtype changes. The branching pattern together with estimates of evolutionary rates suggest that the relapse associated clone typically preexisted years before diagnosis. 15% of tumors present hypermutations at relapse in highly expressed genes with a clear mutational signature. It was found that 11% of recurrent tumors harbor mutations in LTBP4, a protein binding to TGF-β. Silencing LTBP4 in GBM cells leads to TGF-β activity suppression and decreased proliferation. In IDH1-wild-type recurrent GBM, high LTBP4 expression is associated with worse prognosis, highlighting the TGF-β pathway as a potential therapeutic target in GBM.

Glioblastoma (GBM) is the most common and most aggressive type of primary brain tumor in adults. Therapeutic options are limited, consisting of surgery and treatment with radiotherapy plus an oral alkylating agent, temozolomide (TMZ). Despite TMZ's benefits, the extension of patients' survival averages ˜2.5 months, and tumors invariably recur leading to fatal outcome. Recent progress in large-scale sequencing techniques has revealed the genomic landscape of the untreated tumor, yet very few studies have analyzed recurrent GBM, and patient cohorts are limited in size.

The evolution of tumor cells under therapy can be viewed as a Darwinian process of clonal replacement in which treatment ablates vulnerable cells while positively selecting for resistant clones. Studies of spatially distinct tumor fragments indicate that treatment failure is frequently complicated by intratumor heterogeneity (ITH), a common phenomenon in low and high-grade glioma. Mutations of the TP53 gene were recently proposed to mark subclonal heterogeneity of GBM, but a clear pattern of tumor evolution remains elusive. ITH and diversity in evolutionary trajectories preclude the identification of general evolutionary patterns in GBM, especially when only limited cohorts of patients are available for the analysis.

To find genetic markers of progression and to elucidate the diverse evolutionary trajectories by which GBM can occur and recur, performed whole-exome and transcriptome analysis was performed for untreated and recurrent tumors from 114 GBM patients with corresponding matched normal tissue.

### Methods

### Patients and Samples

Recurrent GBM patients were collected from Besta Brain Tumor Biobank (INCB, R001-R019), MD Anderson Cancer Center (MD Anderson, R020-R029), The Cancer Genome Atlas (TCGA, R030-R042), University of California San Francisco (UCSF, R043-R052), Kyoto University (KU, R053-R055), and Samsung Medical Center (SMC, R056-R093, R094-R114).

The specimens in cohort INCB originate from the Besta Brain Tumor Biobank, which is partly funded by the Italian Minister of Health. All patients signed an informed consent for the use of their biological material for research purposes. One case (R012) from this cohort had a history of lower grade glioma prior to the first GBM. All patients were treated by standard Stupp treatment with surgery followed by radiotherapy plus concomitant and adjuvant TMZ.

Samples from cohort MD Anderson were primary and recurrent paired tumor obtained from Henry Ford Hospital in accordance with institutional policies and all patients provided written consent, with approval from the Institutional Review Board (IRB protocol #402). Three cases had a history of lower grade astrocytoma prior to the first GBM (R022/R027/R029). All of the recurrent GBMs had been treated with radiochemotherapy plus TMZ. Cohort TCGA contains TCGA samples, following the publishing protocol of TCGA policies. All of the recurrent GBMs had been treated with chemotherapy or radiation. Six patients were not treated by TMZ (R031/R034-R038). Cohort MD Anderson and TCGA were initially published by Kim et al.

Cohort UCSF contains eight patients (R043-R050) collected from the Neurosurgery Tissue Bank at the University of California San Francisco (UCSF), approved by the Committee on Human Research at UCSF. Two patients (R051/R052) from this cohort were from University of Tokyo hospital and the study was approved by the Ethics Committee of the University of Tokyo. Initial tumors of all patients in this cohort were low-grade gliomas, and their recurrences were secondary GBM. This cohort was initially published by Johnson et al. Cohort KU makes use of data generated by Department of Pathology and Tumor Biology, Kyoto University. Initial tumors of patients from KU were low-grade gliomas, and their recurrences were secondary GBM. Those patents were initially published by Suzuki et al.

Cohort SMC consists of GBM samples from Samsung Medical Center (SMC), Korea, following the prior publication (Kim et al, Cancer Cell 2015, R056-R093) and additional unpublished samples (R094-R114). All samples from SMC had been collected with approval from the Institutional Review Board (IRB file #201004004 and #201310072). Initial tumors from R076-R078/R098/R105/R114 were secondary GBM, with history of low-grade gliomas. Patient R103 had cervical cancer three years prior to the first diagnosis of GBM.

### Sequencing and Mapping

Genomic DNA from initial tumor/recurrent tumor/matched normal blood of patients R001-R016, and recurrent tumor of patients R017-R019 were extracted purified, quantitated, fragmented, quality controlled, and used to create a library of genomic DNA fragments. gDNA fragmentation was performed using the Covaris S220 AFA instrument to reproducibly generate fragments of a precise length, while quality control of both gDNA samples and library fragments (at a later time) was performed using Agilent Bioanalyzer 2100 microfluidic device. Both untreated and treated tumor samples of R009, R011, and R014, plus recurrent samples of patients R017-R019 were sequenced by Agilent V3 50M kit, sequencing 90 bp PE. Mapping files of untreated/normal samples of patients R017-R019 were obtained from TCGA through CG-hub. All other DNA samples from cohort INCB were sequenced by the protocol of Agilent SureSelect XT Human All Exon v4 Kit, PE, 80M reads, 150X on target coverage. High-quality reads of those samples were mapped by BWA to human genome assembly of hg19 with default parameters. All mapped reads were then marked duplication by Picard to eliminate potential duplications. Total RNA of samples in cohort INCB was collected to investigate the transcriptional profiling by mRNASeq using Illumina technology. Upon quantification and quality controls, mRNAs were reverse transcribed to cDNA and a library of fragments was synthesized using Illumina TruSeq mRNA kits. Total RNA depleted of ribosomal RNA of patients R001-R005, R007-R008, R010 and R012 were sequenced by TrueSeq3 stranded prep (Illumina). RNA samples of R006, R009, R017-R019 were sequenced in BGI. All reads were mapped to human genome assembly of hg19 from UCSC genome browser, using a fast splice junction mapper Tophat.

Mapping files of TCGA samples but R039 were downloaded through CG-hub from TCGA. DNA mapping files of cohort UCSF, cohort MD Anderson, cohort KU, and R056-R093 from cohort SMC were all downloaded from European Bioinformatics Institute (EGA) with accession number EGAS00001000579, EGAD00001001113, EGAD00001001213, and EGAD00001001424. Additional samples (R094-R114) from SMC followed the same sequencing protocols as the previous samples in the prior publication (Kim et al, Cancer Cell 2015, R056-R093).

### SAVI2 and Driver Gene Selection

To identify somatic mutations from whole-exome sequencing data of triple samples (normal, initial tumor, and recurrent tumor) of GBM patients, variance-calling software SAVI2 (statistical algorithm for variant frequency identification) was applied based on the empirical Bayesian method. Specifically, the candidate variant list was first generated by successively eliminating positions without variant reads, positions with low-depth, positions that were biased in one strand, and positions containing only low-quality reads. Then the number of high quality reads of forward ref alleles, reverse ref alleles, forward non-ref alleles, and reverse non-ref alleles were calculated in the remained candidate positions to build the prior and the posterior distribution of the mutation allele fraction. Finally somatic mutations were determined based on the posterior distribution of difference of the mutation allele fraction between normal and tumor samples. SAVI2 was able to assess mutations by simultaneously considering multiple tumor samples, as well their corresponding RNA samples if available.

The known driver list used herein was generated by combining GBM drivers from cancer gene census and the previous analysis of primary GBM.

### The Analysis of Loss of Heterozygosity (LOH) and Copy Number Change

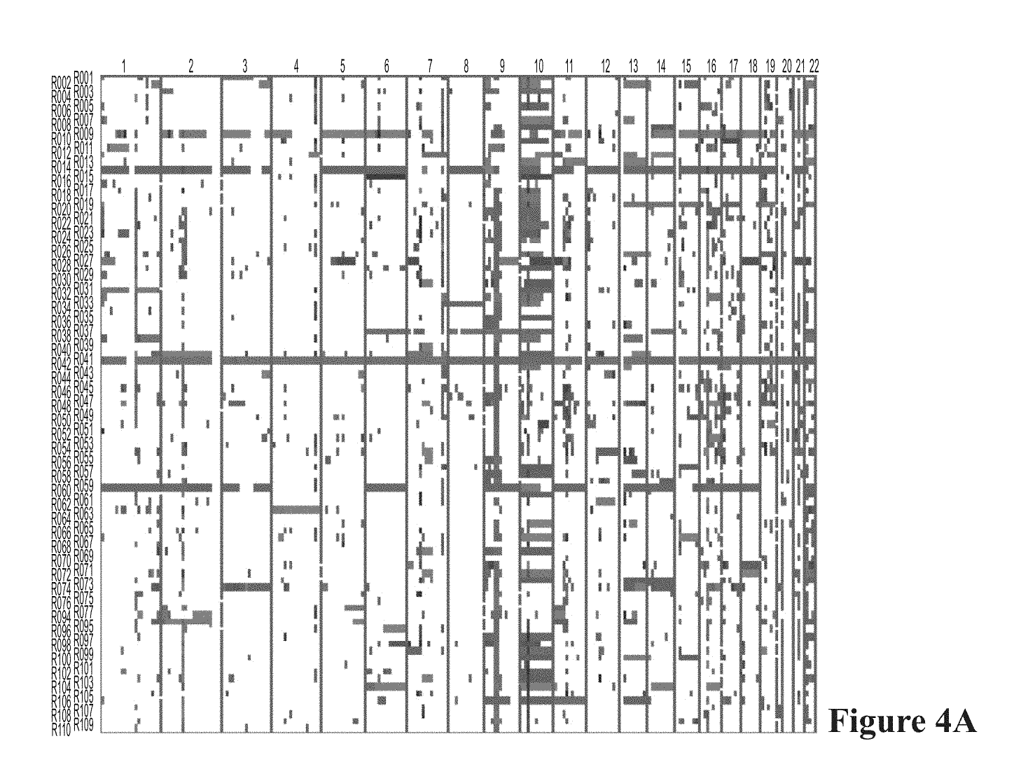

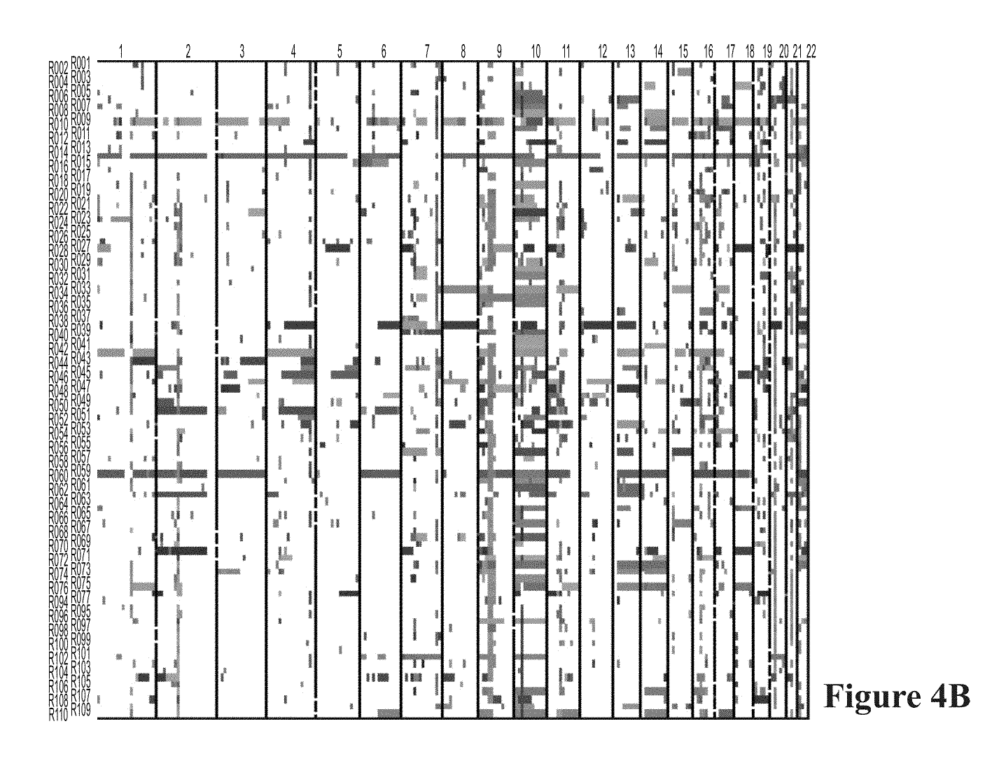

All common dbSNP variants of single samples were extracted to define Zygosity Score (ZS) as ZS=f (1−f). The LOH rate of somatic mutations in a tumor sample was then defined by (1):

$$r = \frac{{\sum }_{i = 1}^{n} {ZS}_{i}^{T}}{{\sum }_{j = 1}^{n} {ZS}_{j}^{N}} \qquad ( 1 )$$

where ZSᵢ^{T} is zygosity score in tumor samples, while ZSᵢ^{N} is that of the normal samples. If r<0.8, it is believed that the corresponding mutation is in a LOH region. Segmentation in FIG. 4 was performed based on CBS algorithm.

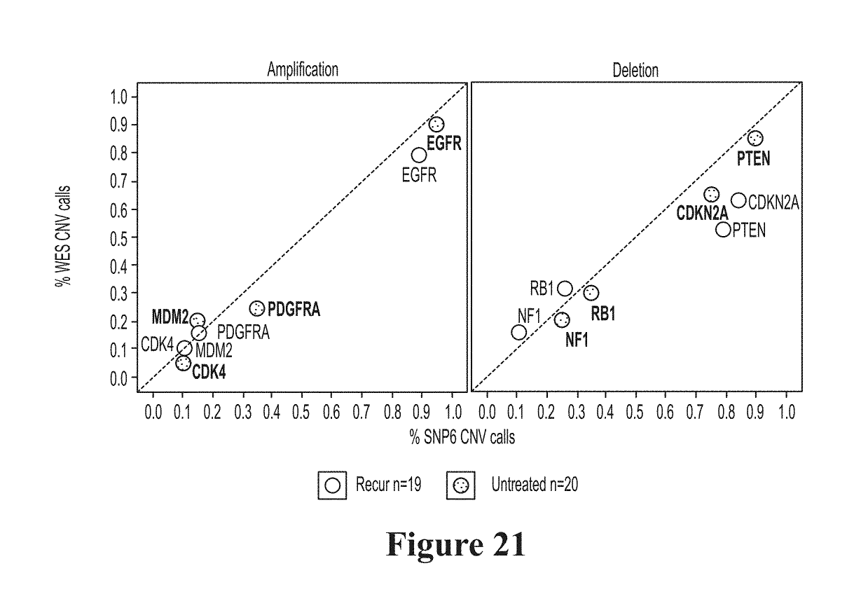

The pipeline of EXCAVATOR was carried out to detect copy number alterations based on whole-exome sequencing data. EXCAVATOR considers mean number of reads per exon, and normalized the data by a three-part normalization procedure to eliminate the bias introduced by GC content, the genomic mappability and the exon size. Segmentation was then performed with a novel heterogeneous hidden Markov model algorithm, heterogeneous shifting level model (HSLM) algorithm, which considers the genomic distance between consecutive exons. To confidently quantify variation arising in whole-exome sequencing (WES) data in each patient's initial and recurrent sample compared to normal data WES CNV calls to SNP array data were calibrated in available samples. FIG. 21 is a graph depicting the calibration of copy number methods. The x-axis indicates the fraction of alterations based on SNP array method, and the y-axis indicates that of the WES-based method. In addition to WES segmentation data was used for TCGA samples from Broad Firehose platform (http://gdac.broadinstitute.org) and when available SNP6 data pre-processed with AROMA (http://www.aroma-project.org) and normalized ArrayCGH obtained from Gene Expression Omnibus (GSE63035). To identify statistical significant regions, GISTIC was applied in initial and recurrent tumors respectively. GISTIC estimated the background rates for each amplification and deletion, and then summarize the input samples to score the significance of copy number altered regions. To integrate mutation and copy number data, MutComFocal was separately performed in initial and recurrent tumor. In the MutComFocal analysis, long proteins (with more than 3500 amino acids), not expressed genes (mutations were not expressed in any samples) and high-synonymous-rate genes (synonymous/non-synonymous>0.2) were not considered.

### Gene Fusion Detection and Structure Rearrangement of EGFR

ChimeraScan was used to generate the starting set of gene fusion candidates. To reduce the false positive rate and nominate potential driving events, the Pegasus annotation and prediction pipeline was applied. The entire fusion sequence was reconstructed on the basis of the breakpoint coordinates and assigned a driver score to each candidate fusion via a machine learning model trained largely on GBM data. All candidates were selected according to three criteria: 1) Pegasus score was >0.5; 2) Either more than 400 span reads or at least two split reads supported fusion; 3) The two fusion partner was apart at least 50 kb.

To check the rearrangement of EGFR, prada-guess-if from PRADA package was applied. PRADA is a RNA sequencing analysis pipeline developed in MD Anderson. Following the definition in Brennan et al. 2013, transcribed allelic fractions of EGFRvIII were defined as the fraction of junction reads between exon1 and exon8.

### Gene Expression Analysis and Expression-Based Subtyping Analysis of GBM Samples

Fragments Per Kilobase of exon model per Million mapped fragments (FPKMs) were calculated by Cufflinks. To eliminate batch effect, gene expression was normalized by calculating Z-score in each batch. The gene expression was assessed by their corresponding Z-scores. ssGSEA was applied to determine the subtype of GBM samples. For each sample, Z-score was used to rank all genes to generate the rnk.file as the input of GseaPreranked software. An enrichment score (ES) was generated for all four subtypes initially defined in Verhaak et al. 2010. The subtype with the maximal ES was selected as a representative subtype of each sample.

### Moduli Space Analysis

Clustering analysis of the patient data was performed as follows. Each phylogenetic tree was represented as a point in the projective evolutionary moduli space, which in this case is a triple (x₁, x₂, x₃) such that x₁+x₂+x₃=1, by taking the raw mutation counts (z₁, z₂, z₃) for the common, initial, and recurrent mutations and normalizing, setting x₁=z₁/(z₁+z₂+z₃). Samples were discarded where any of the mutation counts were missing, leaving 93 points (out of 114 patients). The metric on the evolutionary moduli space was in this case simply the standard Euclidean metric. Note that for purposes of constructing this space, the “branch lengths” of each patient's tree are simply mutation counts, in contrast to the evolutionary analysis described below, which estimates branch lengths in years.

Three clustering algorithms were applied to this metric space: k-means clustering, spectral clustering, and density-based spatial clustering (DBSCAN). The code provided as part of the scikit Python package was then used. For k-means clustering and spectral clustering, the number of clusters was set at three; DBSCAN determines the number of clusters from the data, but the parameters were set to be ε=0.5 and minimum cluster size=5. For spectral clustering, the affinity matrix was computed using the Gaussian kernel applied to the Euclidean distance.

In order to ensure stability of the results, cross-validation was performed using Monte Carlo simulations in which 95% of the data points were sampled without replacement and clustering was performed.

### Tumor Purity Estimation and Cellular Fraction

ABSOLUTE was used to infer tumor purities and ploidy for each WES sample by integrating mutational allele frequencies and copy number calls.

PyClone was run for each sample using default parameters. Briefly, both allele mutations, copy number calls and loh status were integrated for each sample as input to obtain cellular frequencies. Cellular frequencies were then rescaled by median adjustment and used as input for Tumor Evolutionary Directed Graph and Mathematical modeling of tumor evolution.

### Evolutionary Model

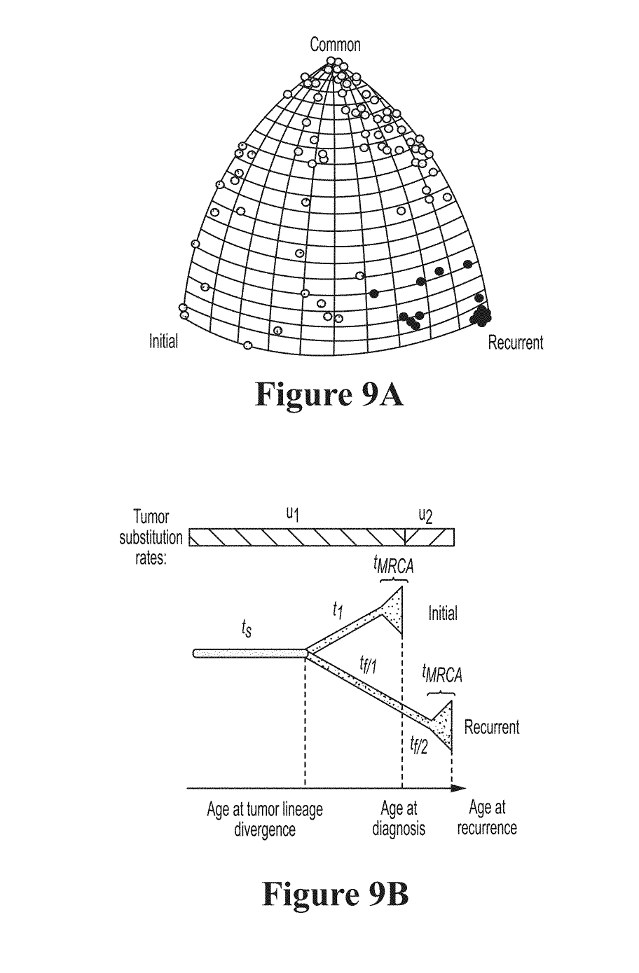

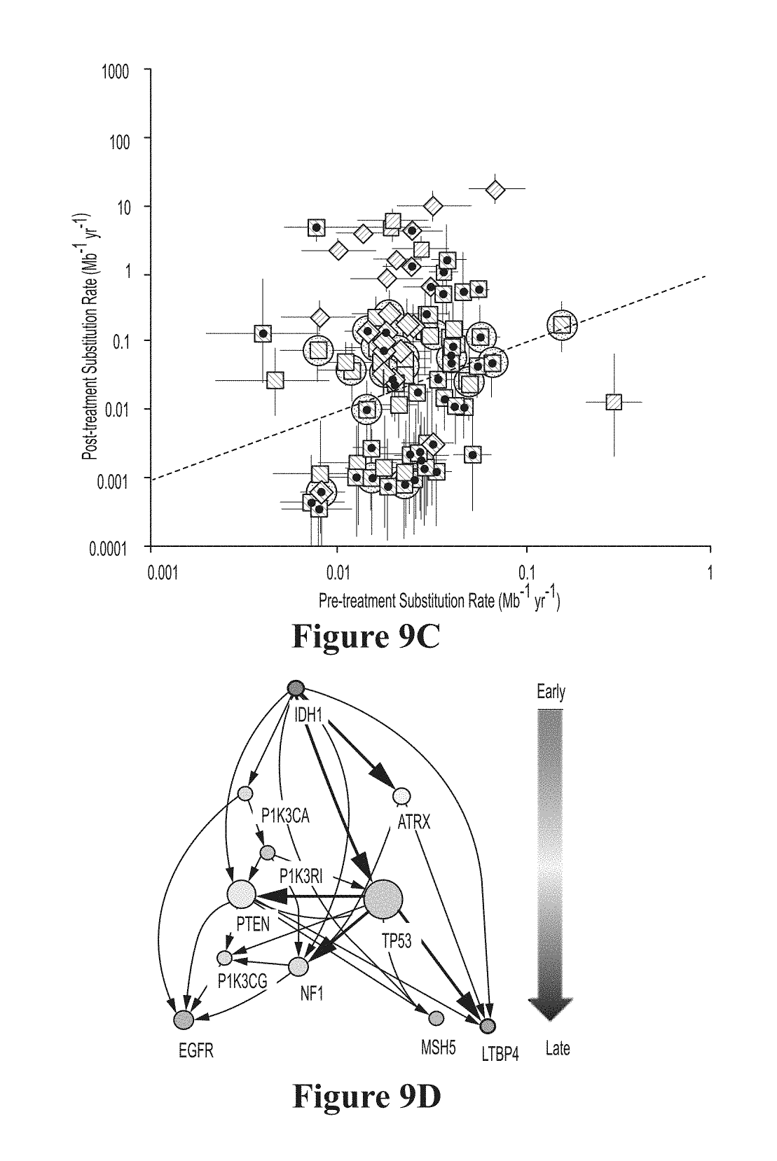

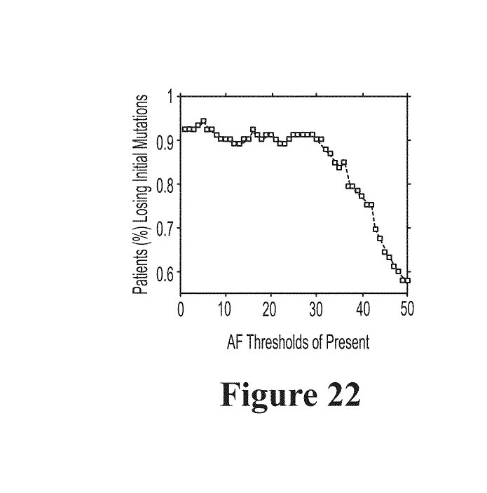

All 92 patients for whom mutations were sequenced in both the initial and recurrent tumor samples were included. To exclude false positives, only variants with an allele frequency of at least 5% were used. Variants occurring at a cellular fraction of at least 95% were classified as clonal in a sample, and others were considered subclonal. FIG. 22 is a graph showing the fraction of patients losing initial mutations. Different allele frequency cutoffs were applied to identify patients with at least one mutation in the initial tumor that is absent from the recurrent tumor. For any presence/absence cutoff below 30%, about 90% of patients lose at least one mutation, suggesting that the tumor at diagnosis is not a direct ancestor of the clone at relapse. In FIG. 22, sensitivity analysis is performed using alternate cutoffs for clonality. Below the abbreviations are as follows: “C” for clonal, “S” for subclonal, and “X” for absent. Clonality status provides evidence for the timing of the mutation: Clonal mutations occur prior to the most recent common ancestor of the sample (defined as occurring within that sample's branch), while subclonal mutations occur after that common ancestor's lineage has bifurcated (defined as occurring within that sample's diversification). Considering both the initial and recurrent samples, there are five mutational patterns that can be explained by a single mutation event in the model depicted shown in FIG. 9B:

CC: The variant occurs clonally in both the initial and recurrent samples. (Mutation event in the shared branch.)

CX: The variant occurs clonally in the initial sample and is absent from the recurrence. (Mutation event in the initial branch.)

XC: The variant occurs clonally in the recurrent sample and is absent from the initial. (Mutation event in the recurrent branch.)

SX: The variant occurs subclonally in the initial sample and is absent from the recurrence. (Mutation event in the initial diversification.)

XS: The variant occurs subclonally in the recurrent sample and is absent from the initial. (Mutation event in the recurrent diversification.)

The other three mutational patterns can be explained by positing at least two mutation events:

CS: The variant occurs clonally in the initial sample and subclonally in the recurrence. (Case 1: Mutation in the shared branch, back-mutation in the recurrent diversification. Case 2: Mutation in the initial branch, same mutation in the recurrent diversification.)

SC: The variant occurs subclonally in the initial sample and clonally in the recurrence. (Case 1: Mutation in the shared branch, back-mutation in the initial diversification. Case 2: Mutation in the initial diversification, same mutation in the recurrent branch.)

SS: The variant occurs subclonally in both the initial and recurrent samples (Mutation in the initial diversification, same mutation in the recurrent diversification.)

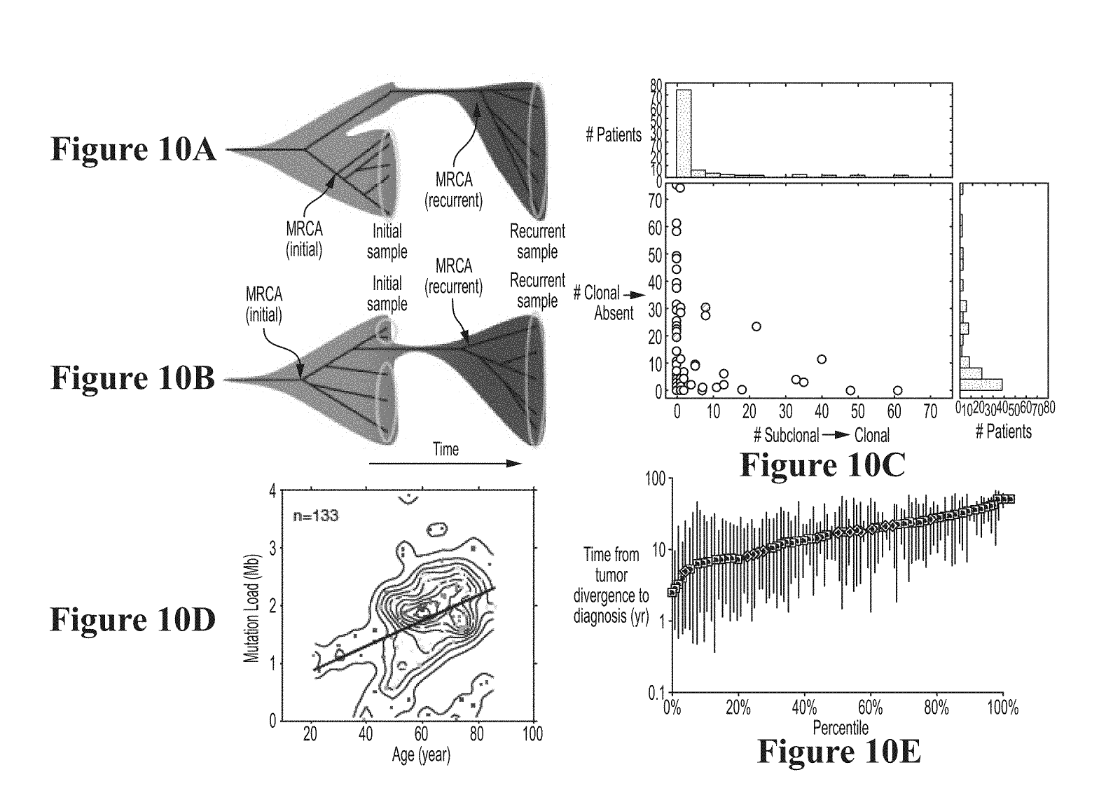

Notably, this model assumes that the initial and recurrent samples are monophyletic—that is, each forms a distinct evolutionary Glade, having diverged from a common ancestor sometime in the past. In reality, they can exhibit more complex evolutionary patterns. For example, the recurrent sample can be nested within the initial Glade (as in FIG. 10B), or the two samples can be evolutionarily intertwined. Of the 92 patients studied, 45 fit the monophyletic model well (see “Assessing Model Fit,” below). Of the remaining 47 patients, many had mutational patterns that could not be explained by the monophyletic model, unless unrealistically many mutations occurred twice, in two separate lineages. As an extreme case, Patient R009 had 58 of 100 mutations appear subclonally in both samples. This mutational pattern suggests that the two samples are evolutionarily interwined. The model does, however, allow for less extreme levels of recurrent- or back-mutation: there are well-fitting patients with up to 23 mutations clonal in one sample and subclonal in the other.

The expected number of mutations of each pattern was computed using a second-order approximation, i.e., the probability that the same mutation occurs (or back-mutates) three or more times is zero. The following parameters are needed for the computation:
Substitution rates:
   Per-site, per-year substitution rates u₁ (pre-treatment) and u₂ (post-treatment);
   Per-site, per-year back-substitution rates v₁ (pre-treatment) and v₂ (post-treatment);
Times:
   Branch lengths, in years: t_{S} (shared branch), 6 (initial sample branch), t_{R1} (recurrence branch prior to treatment), and t_{R2} (recurrence branch after treatment)
   Time between the most recent common ancestor of a sample and the collection of that sample (t_{MRCA}), assumed to be the same for both samples.
The times are constrained so that the age at diagnosis equals i_{S}+t₁+t_{MRCA}, the age at recurrence equals i_{S}+t_{R1}+t_{R2}+t_{MRCA}, and t₁+t_{MRCA}=t_{R1}.
The effective genome length, L. If substitutions are equally probable at all sites, then this parameter simply equals the length of the entire sequenced genome (exome length, 3×10⁷ bp). Since mutations are not necessarily possible at many sites, and since not every mutation is equally probable, the fitted value is typically smaller. Given the same genomic substitution rate (product of L and a per-site rate), a larger value of L decreases the probability that the same mutation occurs twice or is reversed by back-mutation.
The effective sample sizes s_{I} (initial) and s_{R} (recurrence). A larger sample increases the probability that subclonal variants can be found. Given the average exome coverage for most patients (˜150×), the effective samples sizes was capped at 200.

As a first approximation, the initial sample branch length, pre-treatment forward substitution rate, post-treatment substitution rate, and effective genome length are related to the number of clonal mutations by n_{CC}≈u₁Lt_{S}, n_{CX}≈u₁Lt_{I}, and n_{XC}≈u₁Lt_{R1}+u₂Lt_{R2}, where each subscripted n is the number of mutations of a particular pattern and the time t_{MRCA} is ignored. Solving these three equations produces reasonable point estimates for the time t_{I} and the genomic substitution rates u₂L, u₁L. The actual parameter estimates accounted for the number of mutations of all eight patterns and used a negative binomial likelihood for the number of mutations, where the overdispersion of the negative binomial distribution was also fitted. (This negative binomial fit is related mathematically to the notion of gamma rate variation commonly used in phylogenetics.) Each patient was considered separately and a Bayesian MCMC approach was used to obtain posterior distributions for each parameter. The model was implemented using PyStan v2.8.0.2, an interface for the Bayesian inference programming language Stan. A total of 250,000 Hamiltonian Monte Carlo iterations (burn-in of 125,000) was sufficient for convergence in nearly all patients (effective sample sizes >200, \hat{p}<1.001).

### Assessing Model Fit

Patients were deemed to fit the model well if they passed all the following criteria:
MCMC convergence. The effective sample size for all fitted parameters must be at least 200, and the \hat{p} for all fitted parameters must be at most 1.001.
Genome length. The 95th percentile of L must be at least 10⁶. Some patients had surprisingly many subclonal variants shared between the untreated and recurrence samples. These patients were fitted with a short genome length L, as a small “target size” could explain the occurrence of the same exact mutation twice.
Overdispersion. The median overdispersion for the negative binomial distribution must be at most 15.
Similarity of forward- and back-substitution rates. The two rates must not differ by more than a factor of 10, or, if they do, then the p-value corresponding to this difference must exceed 0.05.
No outlier mutation data. All eight mutation pattern counts must lie between the 1st and 99th percentiles of the fitted negative binomial distributions.
Two-hit approximation valid. The Stan code (Supplementary Material 3) computes the probability that a mutation occurs a second time (or back-mutates) along any branch or within either diversification. The 97.5th percentile of each of these probabilities must not exceed 0.14, and the sum of all of these 97.5th percentile values must not exceed 0.5. Larger values would indicate that the same mutation is likely to occur three or more times.

### TEDG Reconstruction

In order to reconstruct the order of events during tumor progression the strategy in Wang et al. 2014 was followed. Genes that were recurrently mutated and expressed in the samples were selected. In hypermutated cases, only mutations of MSH6 and LTBP4 were considered. A mutation that was predicted to be clonal (cellular fraction>0.8) in both initial tumor and recurrent tumor was defined as an early event, while a mutation that was only present (variant allele fraction>5%) in one sample was defined as a late event. To represent the order of clonal mutations, for each sample, directed edges were added to connect early and late events. Then all directed edges from different patients were combined to show a global landscape of GBM evolution. A copy number alteration was defined as clonal if the absolute value of segmean was larger than one. A copy number alteration was defined as present at the threshold 0.5, and as absent at the threshold 0.1.

### Hypermutation Score

Hypermutation (HM) score was defined as

HM=e^{-(∥WMH-wM∥}^{_{F}}⁾−e^{-(∥WMN-wM∥}^{_{F}}⁾

where WM is the weight matrix of the DNA sequence logo of a given sample; WMH is the weight matrix of all mutations in hypermutated sampels; and WMN is the weight matrix of mutations from all non-hypermutation samples.

### Validation of Mutations

The genomic regions surrounding the predicted mutations were amplified using AccuPrime Taq DNA Polymerase High Fidelity (Invitrogen, USA) and the following primers:

MT-ND4
G > A G320E and MT-ND4 ACAT > A TS322T
Fw: 5′-TTACGGACTCCACTTATGACTCCC-3′
and
Rv: 5′-GAGAGAGGATTATGATGCGACTGTG-3′;
MT-ND4
G > GA K92K?
Fw: 5′-GTTCCCCAACCTTTTCCTCCGA-3′
and
Rv: 5′-GCAGTGAGAGTGAGTAGTAGAATGTTTAG-3′;
SYK
C > T P329S
Fw: 5′-CACCACACCCTCTGAACACCTC-3′
and
Rv: 5′-GGATTCGTGAACCAACTCTACAAAC-3′;
SYK G > A G118E
Fw: 5′-CTTCTTTTTCGGCAACATCACC-3′
and
Rv: 5′-TTTGGGCACACAGGGCACATAG-3′;
SYK
G > A R616K
Fw: 5′-ATCTGAACCTGGGGCTAAAACAC-3′
and
Rv: 5′-TTTGCTTTGTGGAGGGTGAGTC-3′;
LTBP4
G > A G1060S
Fw: 5′-GCCCAGCGTTGTGAGAACAC-3′
and
Rv: 5′-TTGGTCCCATCCACCTCCTG-3′;
LTBP4
G > A S35N
Fw: 5′-TGCTGTTGCTGCCGCTCTTC-3′
and
Rv: 5′-CGAACTAACCCCAGGATTCTCTG-3′;
LTBP4
G > A G1415S
Fw: 5′-CCCAGTCTCAGCCTTCAGATTCTC-3′
and
Rv: 5′-AGGAGCCTCAGATTCGCCATAG-3′;
LTBP4
G > A E292K
Fw: 5′-CCCGTAAGAACCCGTGTAGAC-3′
and
Rv: 5′-AAGTCGCCTCCACAATGG-3′;
LTBP4
C > T L446L
Fw: 5′-CAACCCCAGAACCATTCCCC-3′
and
Rv: 5′-TGACGCTCAGGAGACAAAAACTAAC-3′.

The PCR products were purified with ExoSAP-IT (Affymetrix, USA) and subjected to Sanger Sequencing (Macrogen, USA). The amplicons containing the predicted genomic mutations were sequenced using BigDye Terminator Cycle Sequencing Kit v3.1 on the ABI Prism 3730xl DNA Analyzer (Applied Biosystems, USA).

To assess the sensitivity of judging absence of a mutation in one phase that is present in the other phase, 15 variants in the panel that are absent in one of the samples in WES, with median WES depth 117 [10-402] were studied. Using CancerScan, it was found that no read reported the variant in the sample where it was deemed absent by WES, median CancerScan depth 563 [217-1377].

### Cell Culture, Lentivirus Production and Cell Growth Analysis

U87 (ATCC HTB-14) cell line was acquired through American Type Culture Collection. U251 (Sigma, catalogue number 09063001) cell line was obtained through Sigma. Cell lines were cultured in DMEM supplemented with 10% fetal bovine serum (FBS, Sigma). Cells were routinely tested for mycoplasma contamination using Mycoplasma Plus PCR Primer Set (Agilent, Santa Clara, Calif.) and were found to be negative.

Lentivirus was generated by co-transfection of the lentiviral vectors with pCMV-CMV-ors wpMD2.G plasmids into HEK293T cells as previously described (Niola et al. JCI 2013; Carro et al. Nature 2010). shRNA sequences for LTBP4 are:

shRNA-1:
CAACCGGCTTTGAAAGAGTTACTCGAGTAACTCTTTCAAAGCCGGTTG;
shRNA-2:
CCCAGACTTAGGTCCACCTTACTCGAGTAAGGTGGACCTAAGTCTGGG

After infection cells were selected with Puromycin (Sigma) at concentration of 2 mg/ml for 48 h. Cells were analyzed by western blot, qRT-PCR and growth assay 3 days later.

Evaluation of cell growth was performed using the MTT assay. Cells were plated at density of 2.5×10³ cells/well into 96 well plates in 6 replicates and allowed to adhere for 24 h. Viability was assessed daily by adding MTT ((3-[4,5-dimethylthiazol-2-yl]-2,5-diphenyltetrazolium, Sigma 5 mg/ml in PBS). Following 4 h incubation period, medium was removed and formazan crystal were solubilized with acidic isopropanol (0.1 N HCl in absolute isopropanol. The absorbance at 550 nm was measured with a plate reader.

### RT-PCR

Total RNA was prepared with Trizol reagent (Invitrogen) and cDNA was synthesized using SuperScript II Reverse Transcriptase (Invitrogen) as described (Carro et al. Nature 2010; Zhao et al. Nature Cell Biol 2008). The quantitative RT-PCR was performed with 7500 Real-Time PCR system, using SYBR Green PCR Master Mix from Applied Biosystem. Primers used in qRT-PCR are:

LTBP4
Fw 5′_ACCAGTCATTGTGCCCTCAC_3′
and
Rv 5′_ACATTCGTCCACGTCTCTCC_3′;
ID1
Fw 5′_CGCATCTTGTGTCGCTGAAG_3′
and
Rv 5′_GAGACCCACAGAGCACGTAA;
ID2
Fw 5′_TATTGTCAGCCTGCATCACCAG_3′
and
Rv 5′_GGAATTCAGAAGCCTGCAAGGA_3′;
GADD45A
Fw 5′_AGCAGAAGACCGAAAGGATGG_3′
and
Rv 5′_TACACCCCGACAGTGATCGT_3′;
RHOB
Fw 5′_GTCATTCTCATGTGCTTCTCGG_3′
and
Rv 5′_ATGATGGGCACATTGGGACAG_3′.

Results are presented as the mean±s.d. of three independent experiments each performed in triplicate (n=9). Statistical significance was determined by using unequal variance t-test (two-tailed).

### Western Blot

Cells were lysed in RIPA buffer (50 mM Tris-HCl, pH 7.5, 150 mM NaCl, 1 mM EDTA, 1% NP40, 0.5% sodium dexoycholate, 0.1% sodium dodecyl sulphate, 1.5 mM Na₃VO₄, 50 mM sodium fluoride, 10 mM sodium pyrophosphate, 10 mM β-glycerolphosphate and EDTA-free protease inhibitor cocktail (Roche)). Lysates were cleared by centrifugation at 15,000 r.p.m. for 15 min at 4° C. Protein samples were separated by SDS-PAGE and transferred to nitrocellulose membrane. Membranes were blocked in TBS with 5% non-fat milk and 0.1% Tween20, and probed with primary antibodies. Antibodies and working concentrations are: LTBP4 (1:200, sc-393666) obtained from Santa-Cruz Biotechnology)

### Gene Fusion Validation

For validation of fusion transcripts and RT-PCR assays were performed. Total RNA was extracted from the tissues by AllPrep DNA/RNA Mini kit according to the manufacturer's instructions (Qiagen). The total RNA (0.5 μg) was reverse transcribed to synthesize template cDNA by a random primer using the SuperScriptIII First-Strand System (Life Technologies), and 20 μl synthesized cDNA was diluted 10 times with DW. For RT-PCR, EzWay Taq PCR MasterMix (Komabiotech, KOREA) and 5 μl synthesized cDNA as template were used. Thermal cycling was carried out under the following conditions: 1 min at 95° C. followed by 30 cycles of 30 sec at 95° C., 30 sec at 55° C., 30 sec at 72° C. The primer pairs used in this experiment were designed to make the amplification product including the breakpoints of the fusion genes. PCR products were analyzed by agarose gel electrophoresis. The primers were summarized as shown in Table 1.

TABLE 1

| Position | | |
|---|---|---|
| of primer | Name | sequence |
| BTRC exon 1 | BTRC-MGMT-F1 | TATGTGCTCTATGCCCAGGTC |
| and 2 | | |
| MGMT exon 1 | BTRC-MGMT-R1 | GTCCAGTGTGGTGCGTTTCA |
| and 2 | | |
| BTRC exon 2 | BTRC-MGMT-F2 | TGCCTGTATAACCCAGGGAC |
| MGMT exon 2 | BTRC-MGMT-R2 | CCCTTGCCCAGGAGCTTTATT |
| NFYC exon 2 | NFYC-MGMT-F1 | TCCTGAGCAGAGTTGTCGAG |
| MGMT exon 1 | NFYC-MGMT-R1 | GACCCTGCTCACAACCAGAC |
| and 2 | | |
| NFYC exon 2 | NFYC-MGMT-F2 | CCCAGCAAAGCCTACAGTCG |
| MGMT exon 2 | NFYC-MGMT-R2 | TTATTTCGTGCAGACCCTGCT |

### Results

### Longitudinal Mutational Landscape of GBM Identifies Recurrence-Specific Genetic Alterations

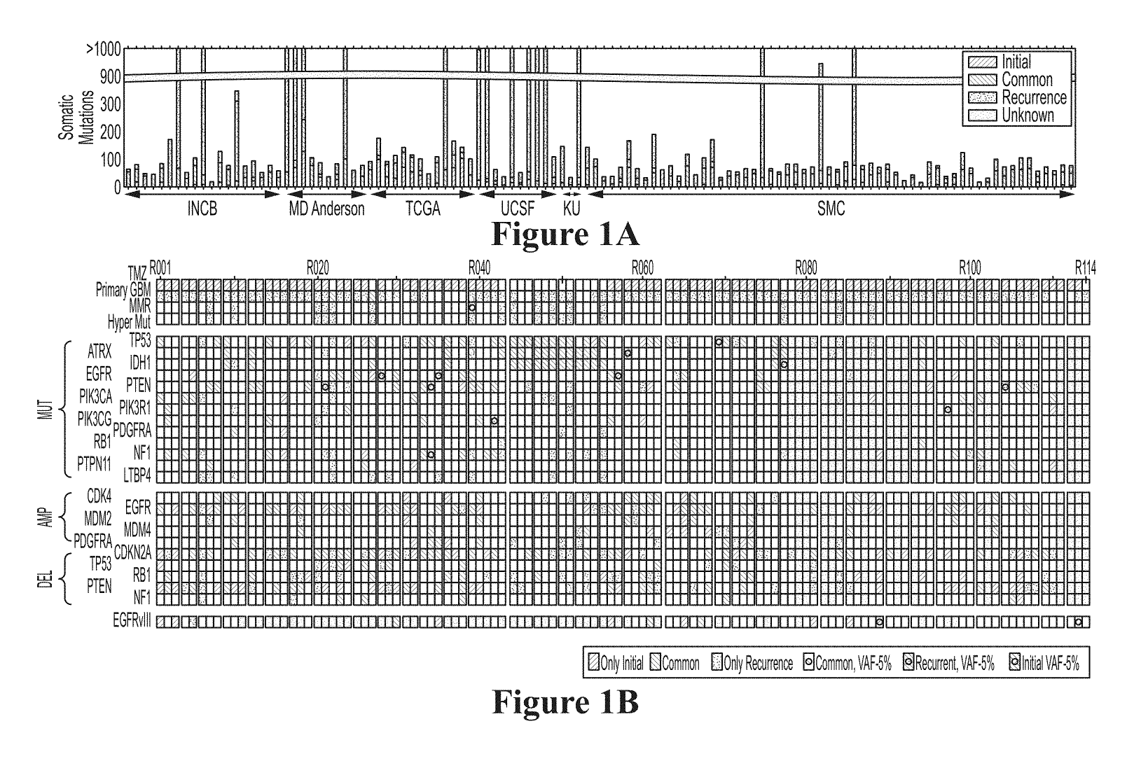

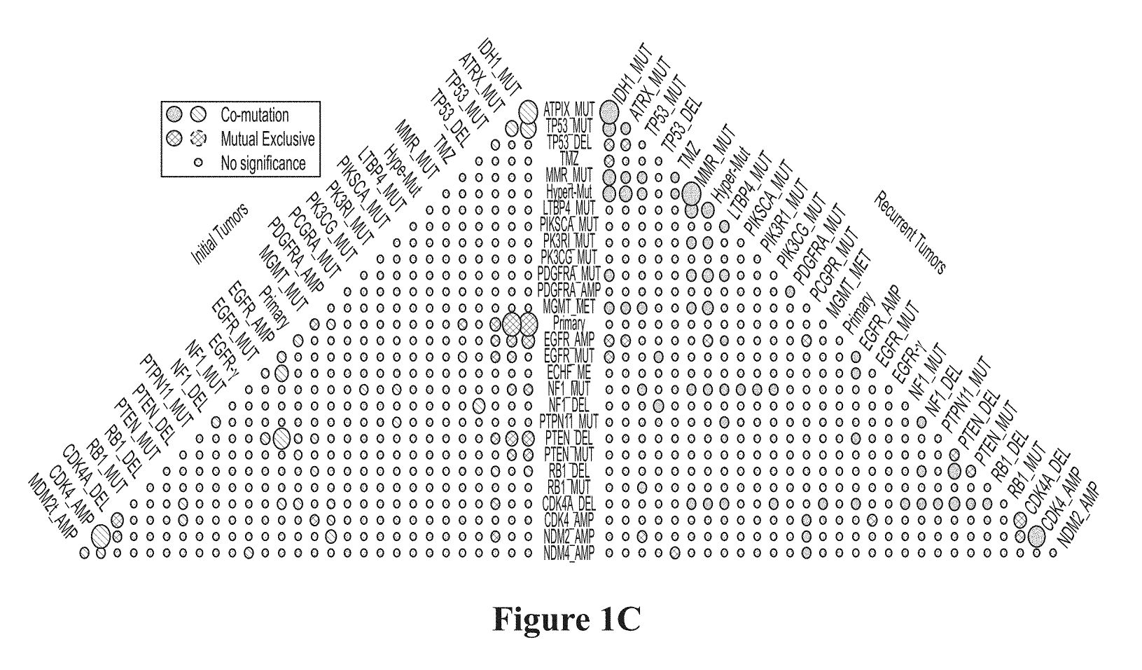

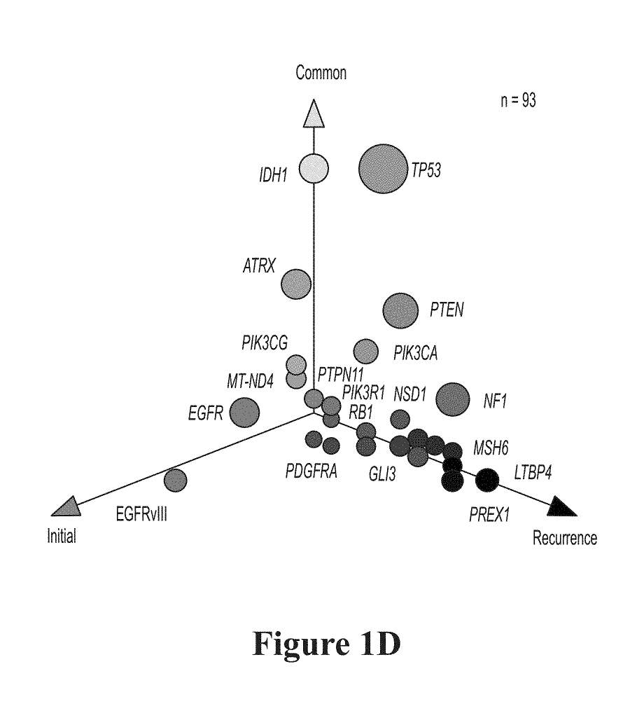

To elucidate the mechanisms driving the evolution of high-grade glioma under therapy, 293 whole-exomes and 141 transcriptomes from longitudinal tumor/matched normal samples in 114 GBM patients were analyzed. FIG. 1A is a graph depicting the number of somatic mutations (Single Nucleotide Variants and small INsertions/DELetions) in 114 Patients from six sources (Instituto Neurologico C. Besta, MD Anderson Cancer Center, The Cancer Genome Atlas, University of California San Francisco, Kyoto University, and Samsung Medical Center). All somatic mutations called by SAVI2 with allele frequency >5% were considered in this analysis. Common somatic mutations from Patients R078-R082 were unknown due to the lack of normal DNA. The initial and recurrence exclusive mutations were calculated based on the difference between initial and recurrent tumor DNA. Tumor DNA of Patient R083-R093, R102, R111-R114 were not complete. The somatic mutations of these patients were estimated based on RNA sequencing.

Recurrent GBM patients (89 diagnosed with primary GBM) were collected from Istituto Neurologico C. Besta (INCB, R001-R019), MD Anderson Cancer Center (R020-R029), The Cancer Genome Atlas (TCGA, R030-R042), University of California San Francisco (UCSF, R043-R052), Kyoto University (KU, R053-R055), and Samsung Medical Center (SMC, R056-R114). Whole-exome triplets of initial tumor sample, recurrent tumor sample, and normal genomic DNA were sequenced from 93 patients. Transcriptomes of initial and recurrent tumor were sequenced from 65 patients. All but 14 patients received standard treatment, including TMZ. Greater than 200 fold mean target coverage was achieved in 84% of samples (246 out of 293). On average, 76% of coding bases within the exome were covered by at least 100 high-quality reads.

To identify somatic single nucleotide variants (SNVs) as well as short insertions and deletions (INDELs), the variant-calling software SAVI2 was used. Only those somatic variants with mutant allele frequency of 5% or more were included. From these variants 40 mutations from the INCB cohort were selected for validation. Sanger sequencing successfully validated 98% (39/40) of the mutational calls as well as changes in allele frequency between untreated and recurrent tumor. Untreated tumor samples harbor an average of 60 somatic mutations. Recurrent tumor samples have 585 somatic mutations on average, but this figure is unrepresentative due to the presence of 17 patients (6 primary GBM and 11 secondary GBM) with hypermutated recurrent tumors (>500 mutated genes per tumor). The remaining non-hypermutated tumors have only 50 mutations on average. All hypermutated tumors originated within TMZ treated patients. 16 out of 17 hypermutated samples gained mutations in genes coding DNA mismatch repair proteins (MSH6, MSH2, MHS4, MSH5, PMS1, PMS2, MLH1, and MLH2). FIG. 1B is a graph illustrating the clinic and genetic profile of patients. TMZ indicates Temozolomide; MMR represents mismatch repair pathway (MSH6, MSH2, MSH4, MSH5, PMS1, PMS2, MLH1, MLH3 were considered). Hyper Mut represents hypermutation. MUT indicates somatic non-synonymous mutations with allele frequency >5% in at least one sample. AMP/DEL indicates copy number change with segmentation mean >0.5, computed either by SNP/CGH array data or by whole-exome sequencing data.

Mutations found in the initial and recurrent samples were compared for each of the 93 patients for whom whole-exome triplets were available. Appearance of the same mutation in both the initial and recurrent samples for a patient suggests that the mutation originated relatively early in that patient's tumor development, while appearance only in one sample suggests that the mutation could have originated after the clonal lineages leading to the two samples diverged. The mutations occurring in only one of a patient's two GBM samples outnumber the common ones in more than half of all patients (57%, 53/93) (FIG. 1A: single-sample mutations versus shared mutations). Next, pairwise co-occurrence and mutual exclusivity of genomic/clinic features were assessed across all patients. FIG. 1C is a graph providing a Pyramids plot highlighting the correlation between different features. A hypergeometric test was performed for each pair of elements by considering initial and recurrent tumors separately. The size of the circle indicates the significance level of the correlation. Any associations with p-value<0.1 were illustrated in this plot. In addition to previously reported association shown in FIG. 1C, a number of significant associations not previously reported for GBM were observed that were exclusive to recurrence. These associations include co-occurrence of MGMT promoter methylation and hypermutation (p-value=4×10⁻³, Fisher's exact test, only TMZ treated patients), co-deletion of RB1 and PTEN (p-value<10⁻⁴, Fisher's exact test); and co-mutation of NF1 and TP53 (p-value=10⁻², Fisher's exact test).

Overall, the mutational analysis reveals both known and potentially novel driver gene mutations in GBM. Mutations in known drivers of GBM were observed, including TP53, PTEN, EGFR, PIK3CA, ATRX, IDH1, PIK3R1, and PDGFRA with similar frequency in both untreated and recurrent tumors. FIG. 1D is a graph providing a 3-D bubble plot illustrating the mutation frequency of somatic non-synonymous mutations in exclusively initial (left axis), exclusively recurrence (right axis), and in common (upper axis). 93 patients with exome-sequencing data in matched normal, initial tumor, and recurrent tumor were considered in this analysis. Hotspot mutations were also identified in unreported potential driver genes in GBM. In particular, seven patients had PTPN11 nonsynonymous mutations (SHP2 protein) in the first SH2 and PTP domains with a similar distribution to what has been found in juvenile myelomonocytic leukemia. A few genes appear exclusively mutated and expressed in recurrent tumors, including LTBP4 (10/93), DNA mismatch repair gene MutS Homolog 6, MSH6 (8/93), PRDM2 (10/93), and IGF1R (9/93). Interestingly all eight cases with mutations in MSH6 occurred in hypermutated recurrences (p-value<10⁴, Fisher's exact test), and three of these cases include nonsense mutations in the gene, indicating that loss of function of MSH6 is related to genomic hypermutation in GBM. This finding is consistent with previous observations of the induction of a hypermutant genotype following treatment of glioma. Recurrence-only mutated gene LTBP4 has been reported to be an activator of TGF-β signaling by promoting the assembly, secretion and targeting of sites where TGF-β1 is stored and/or activated. Disruption of this gene causes abnormal lung development, cardiomyopathy, and colorectal cancer in mice.

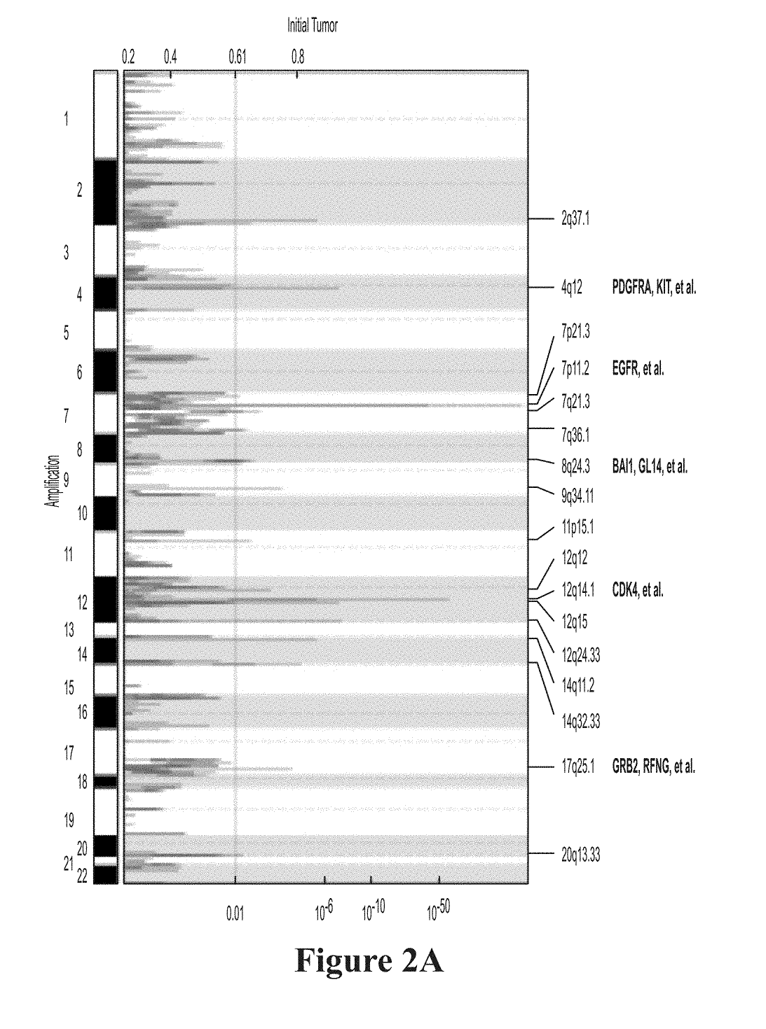

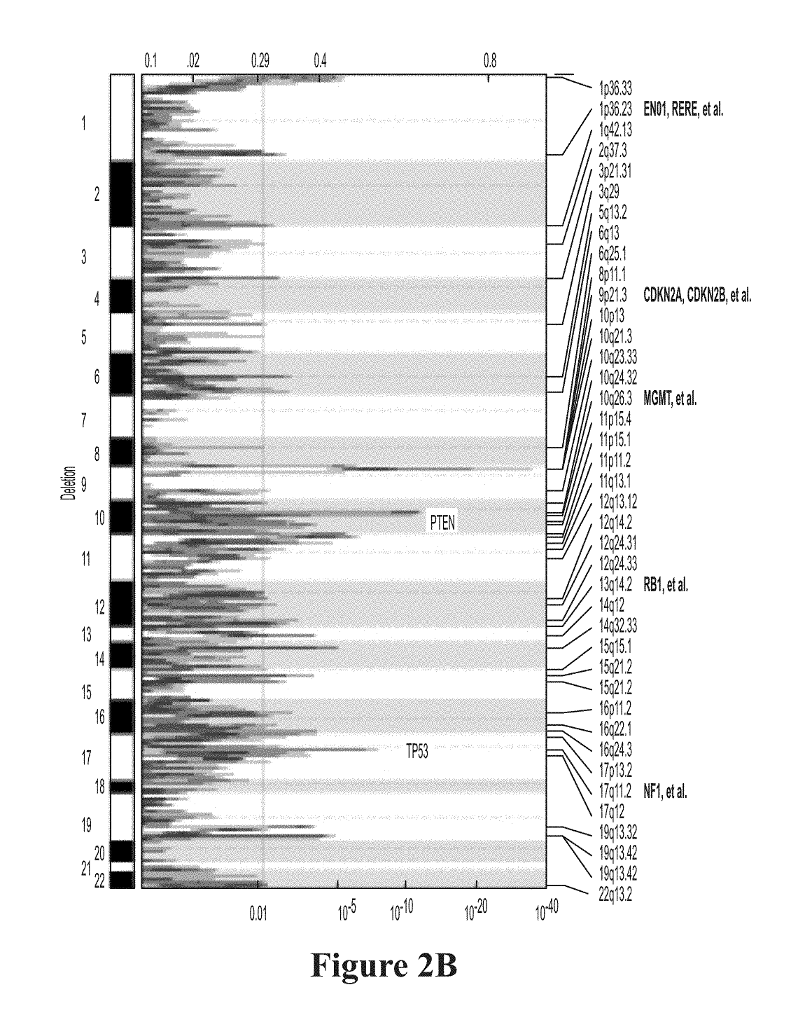

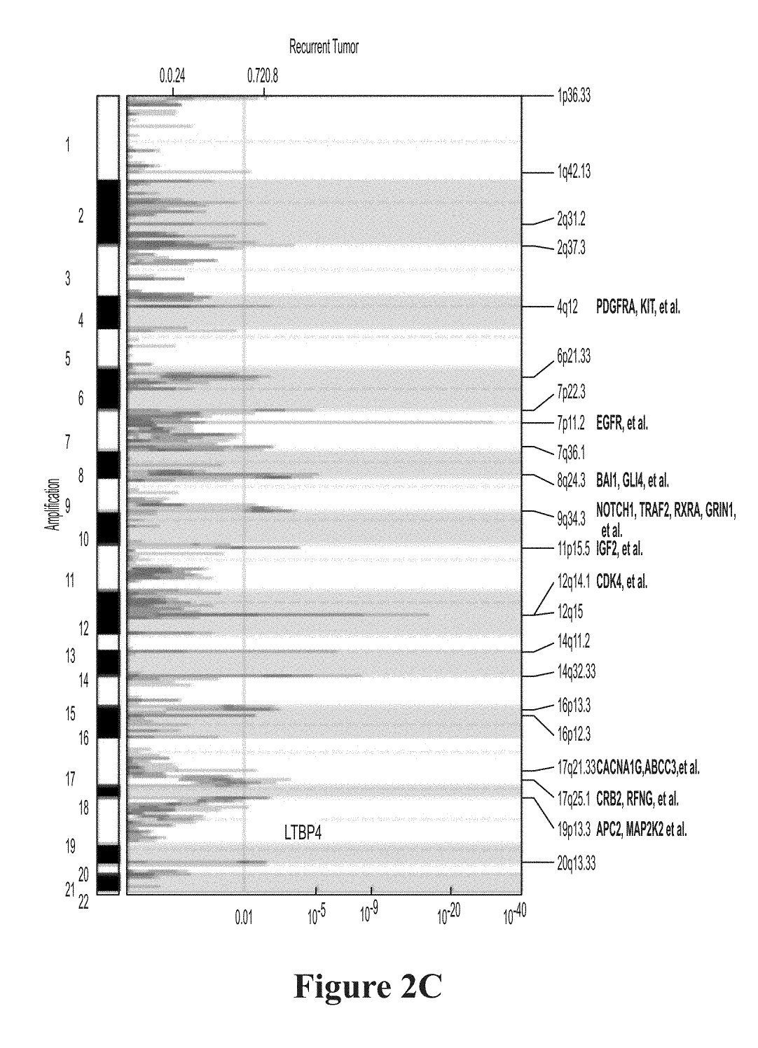

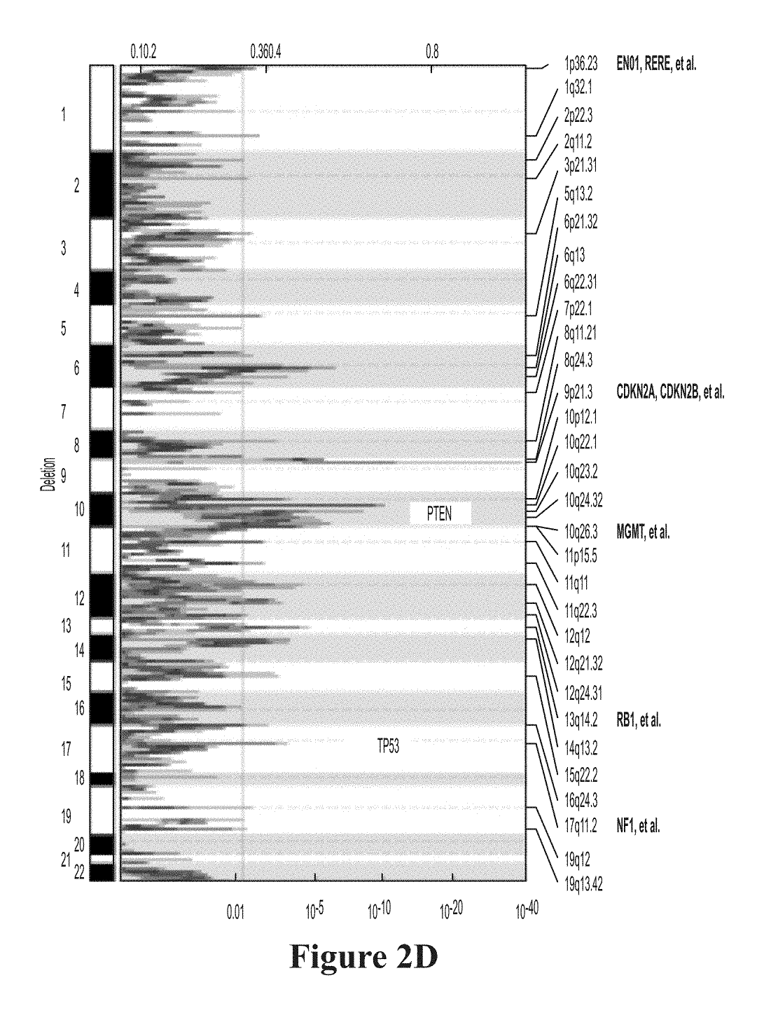

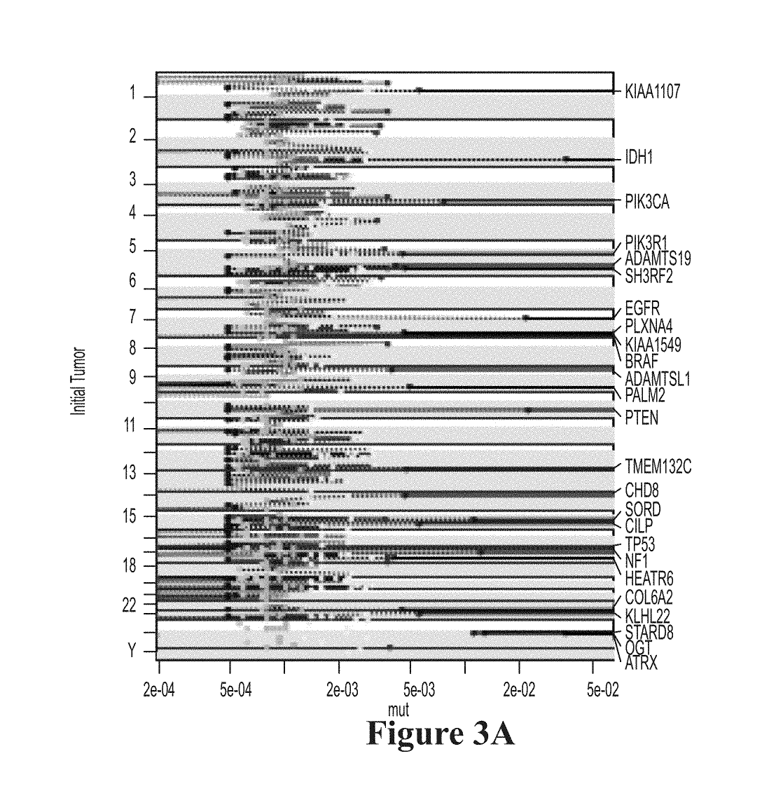

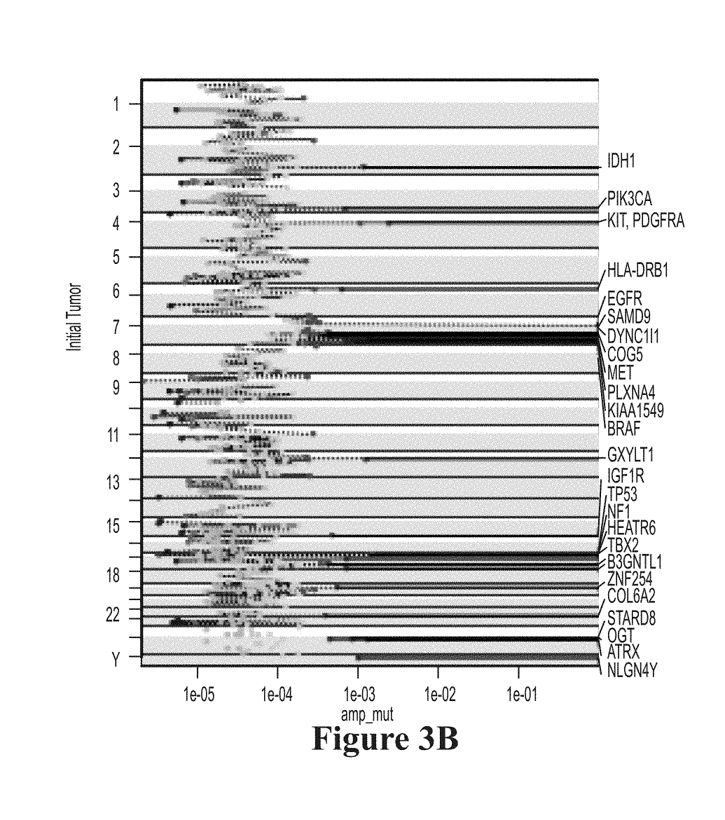

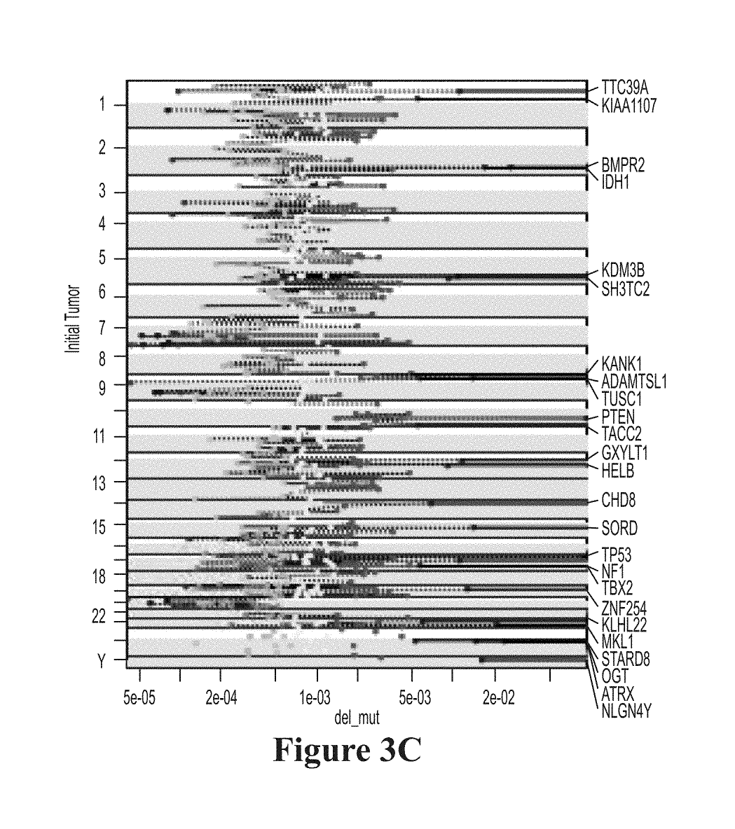

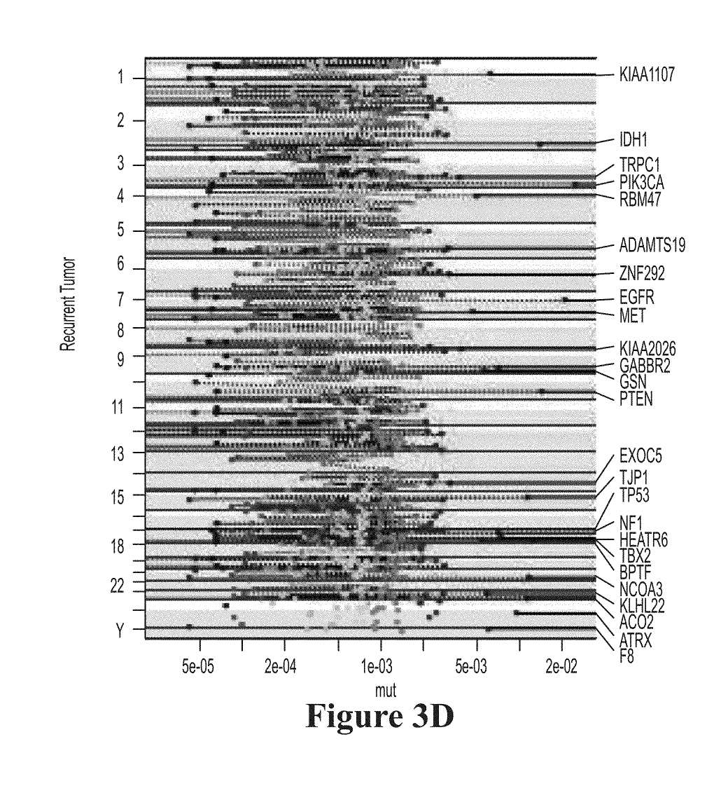

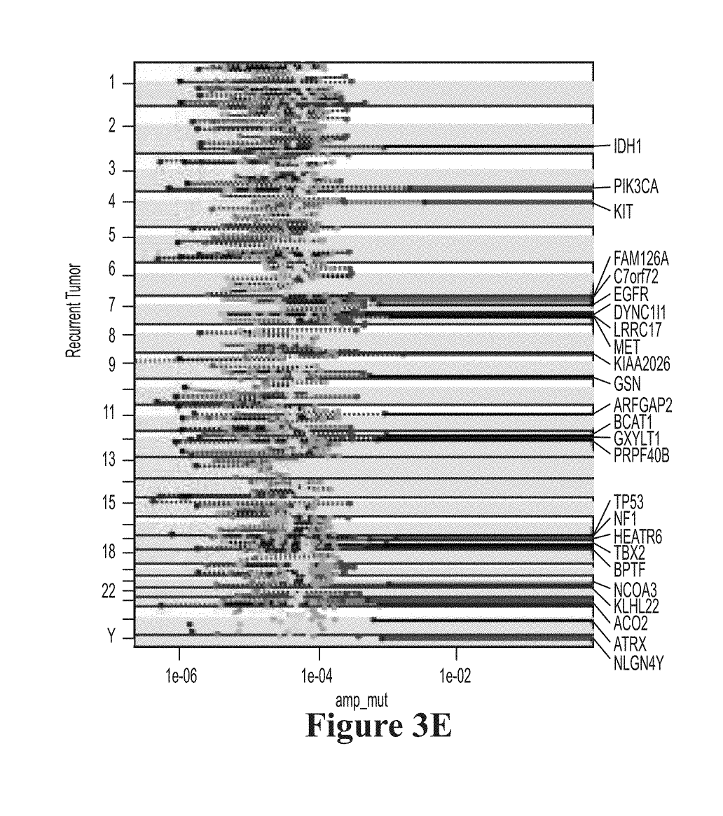

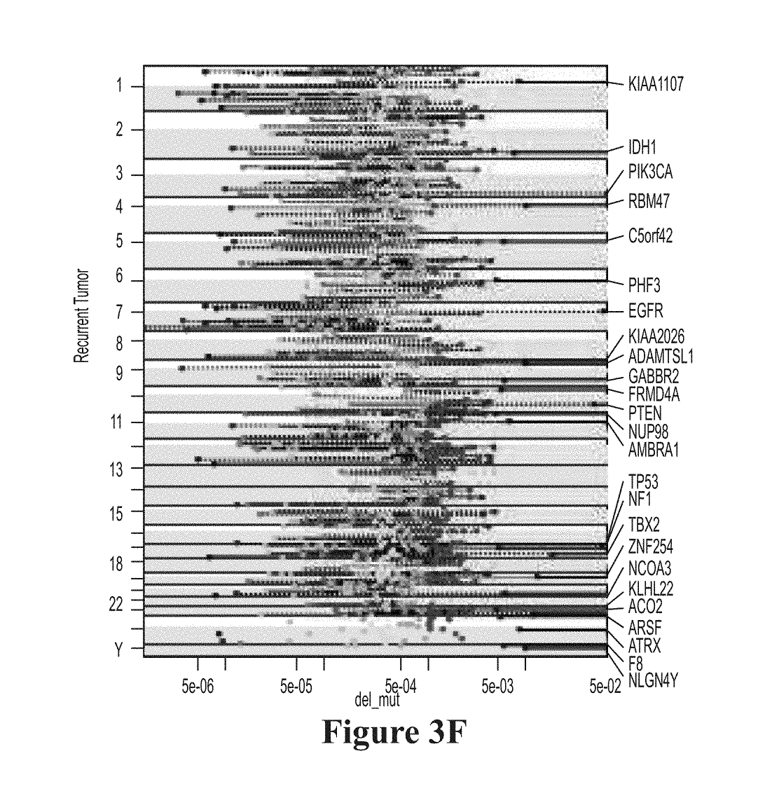

To explore copy number variations (CNVs) of initial and recurrent GBM, recurrence-based analysis, GISTIC2 (FIG. 2) and MutComFocal (FIG. 3) were applied. FIGS. 2A-2D show GISTIC2 qplots of tumors. FIGS. 2A-2B are graphs providing qplots of initial tumors. FIGS. 2C-2D are graphs providing qplots of recurrent tumors. Copy number segmentation files were generated by EXCAVATOR base on Whole Exome Sequencing data. The resulting seg files (genomic intervals), together with the union of whole exome probes from a different platform were used in GISTIC version 2.0.22. FIGS. 3A-3F are graphs providing MutComFocal reports of frequently mutated, amplified, and deleted genes in initial and recurrent GBM samples. The analysis was based on selected mutations and WES-based copy number alteration data. Copy number alterations were found in several well-known GBM drivers. EGFR amplification, which is frequently co-occurrent with EGFR SNVs and EGFRvIII, was observed in 42% of initial tumors (44/104) and 34% of recurrent tumors (35/102), whereas CDK4 amplification was detected in 19% of both initial and recurrent samples (20/104). Deletions in CDKN2A were the most frequent copy deletion in 47% (49/104) of initial samples and 52% of recurrent tumors (53/102). PTEN displayed a similar prevalence of loss in initial 37% (38/104) and recurrent 34% (35/102) samples.

A zygosity score (ZS) was defined to identify regions of loss of heterozygosity (LOH). FIGS. 4A-4B are graphs providing loss of heterozygosity (LOH) analysis based on WES data. FIG. 4A represents LOH regions in initial tumors. FIG. 4B represents LOH regions in recurrent tumors. The median ZS of a normal diploid chromosome is expected to be near to 0.25. To identify potential tumor suppressors associated to a two-hit mechanism, genes with point mutations in regions with LOH in non-hypermutated recurrent tumors were analyzed. This analysis recapitulated known tumor suppressors in GBM, including TP53 (14/78 samples), PTEN (9/78), and NF1 (3/78), and LOH encompassing inactivating mutations in other genes not previously reported in GBM including APC (R876*).

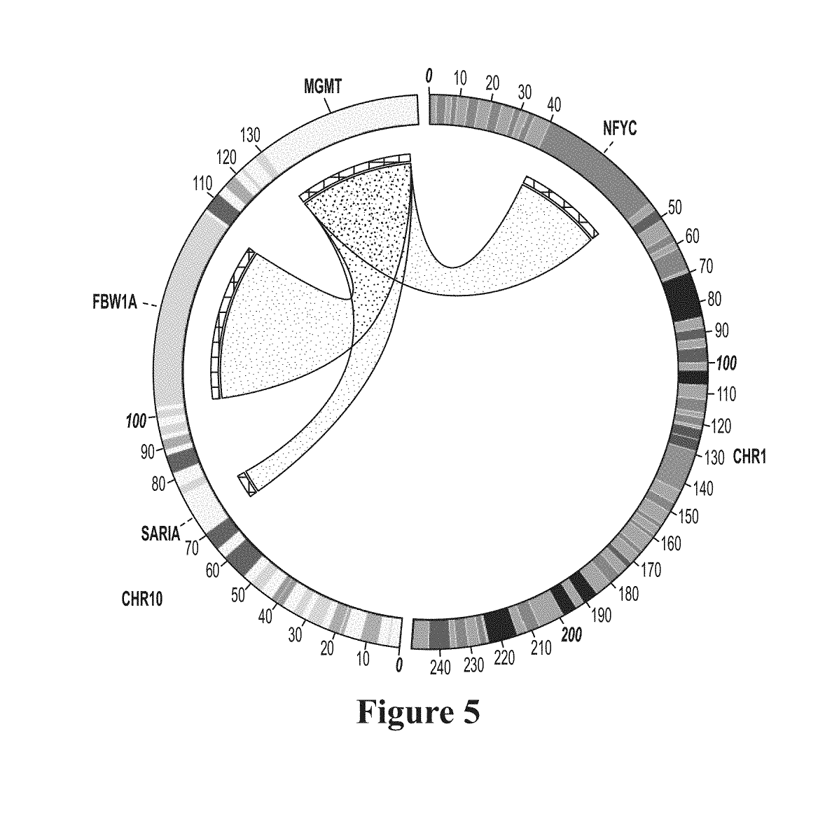

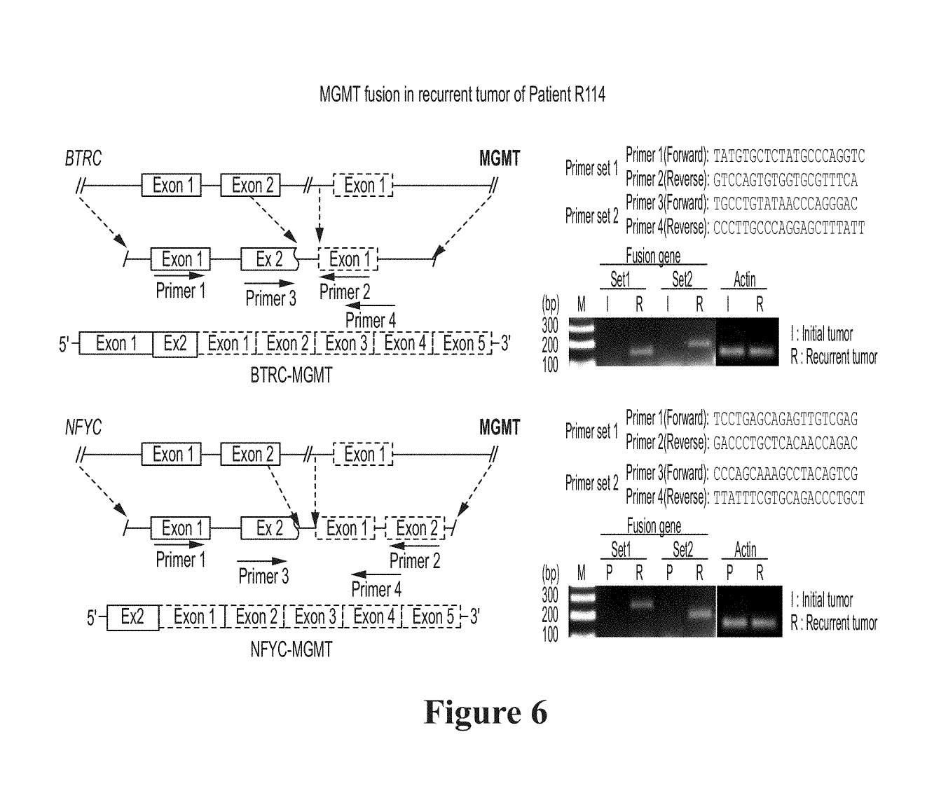

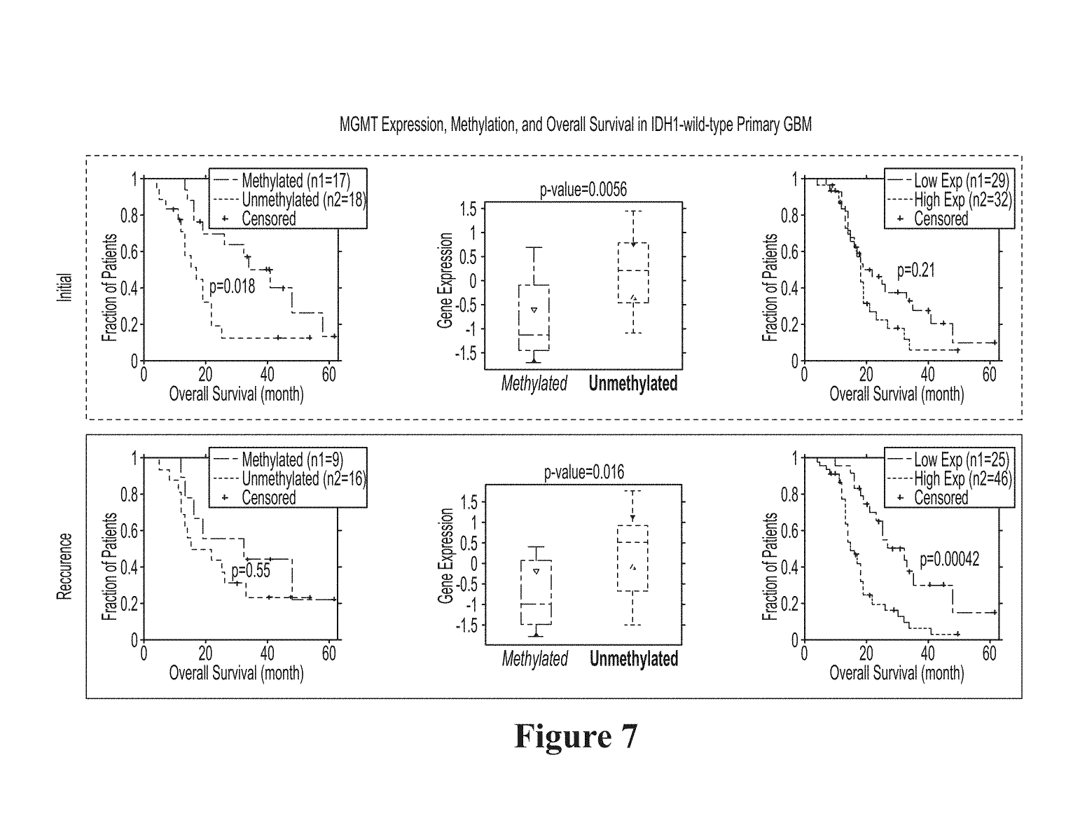

Gene fusions reported as recurrent alterations in GBM were found, such as FGFR3-TACC3 and EGFR fusions with multiple partners. FGFR3-TACC3 fusions were highly expressed in both the untreated and matched recurrent tumors, thus confirming the clonal nature of these fusion events. Rare fusions involving other Receptor Tyrosine Kinase (RTK)-coding genes such as PDGFRA, MET, and ROS were also found. Interestingly, two patients harbored in-frame gene fusions involving MGMT at relapse. FIG. 5 is a graph providing circos plot of MGMT fusions. Patient R114 harbors two highly expressed in-frame fusions NFYC-MGMT, BTRC-MGMT and patient R056 at recurrence presents the fusion SAR1A-MGMT. Of particular significance, these three fusion transcripts carry the same breakpoint in the MGMT gene, and the reconstructed open reading frame preserves the methyl-transferase and DNA binding domain. The fusion transcripts were further validated by RT-PCR. FIG. 6 is a graph depicting experimental validation of MGMT fusion in patient R114. Solid black arrows indicate the position of the fusion genome primers, which generate fusion-specific PCR products. MGMT is a gene that encodes for an O₆-methylguanine-DNA methyltransferase and epigenetic silencing of this gene has been associated with longer overall survival in GBM patients under therapy. Consistently, MGMT methylation at diagnosis predicts longer survival (p-value=0.018). A high correlation between MGMT methylation and expression both at initial and at relapse (p-value=6×10⁻³ in initial and 0.016 in relapse) was also observed. FIG. 7 is a graph showing MGMT expression, methylation, and overall survival in IDH1 wild type primary GBM. P-values of the survival analyses were calculated based on logrank test, and p-values of the boxplot were based on ranksum test. Gene expression level of MGMT was measured by Z-score based on RNA-seq data. High expression means Z>0, and low expression means Z<0. At recurrence, but not in the initial tumor, low expression of MGMT is significantly related to better prognosis (p-value=4×10⁻⁴).

### Hypermutation Related to Temozolomide

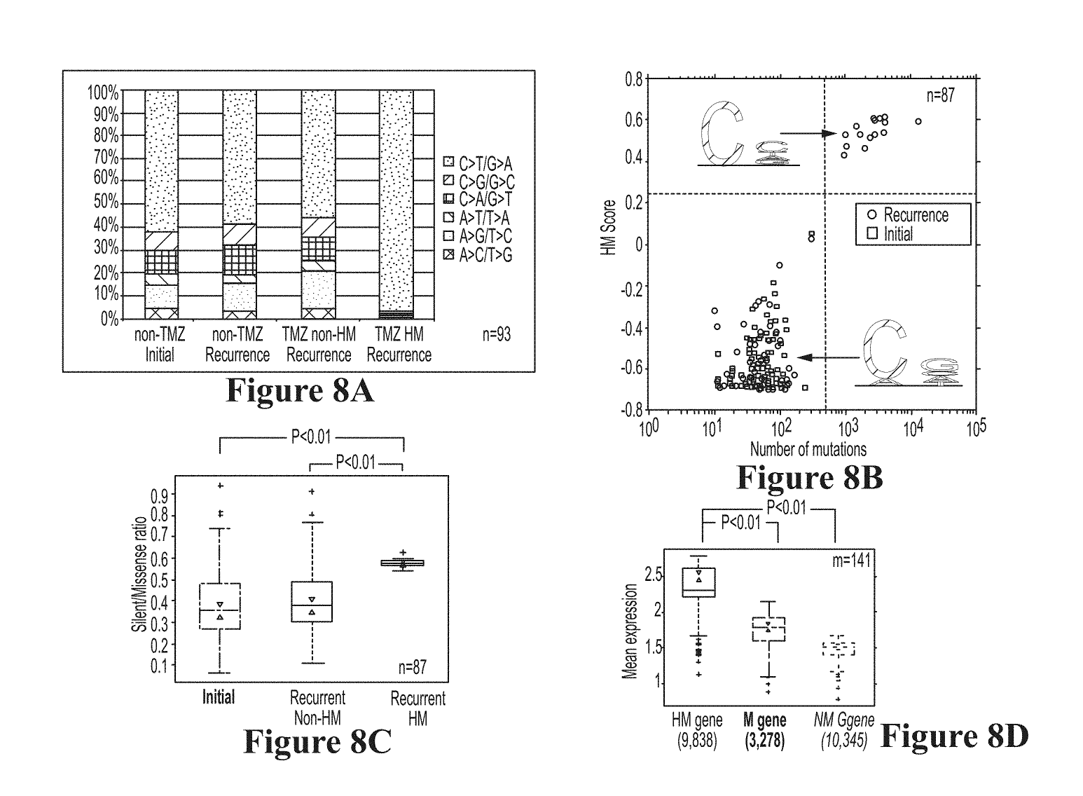

As indicated in FIG. 1A, 17% of TMZ treated GBM patients (17/100) relapsed with hypermutated tumors, yet there is no incidence of hypermutation in non-TMZ treated patients (0/14). The median survival of hypermutated IDH1-wild-type primary GBM patients is 24 months, a slight increase from other IDH1-wild-type primary GBM patients (18 months). The gain of mutations in the mismatch repair pathway as well as the accompanying hypermutations in glioma patients after treatment has been reported before but the pattern of the hypermutated genes and the mechanism causing the mutations remain unclear. To better explain patient mutational variation, all mutations were grouped into four types: those identified in recurrent samples without TMZ, those in untreated tumors, those in TMZ-treated but non-hypermutated cases, and mutations in TMZ-treated hypermutated cases. Hypermutated recurrent tumors are highly enriched with C>T (G>A) transitions. FIG. 8A is a graph showing the fraction of different types of nucleotide changes that are related to Temozolomide (TMZ). In this analysis, 93 patients with trios of normal, initial, and recurrent DNA data were considered. To identify additional markers of hypermutation 10 bp of DNA sequence was extracted from the coding strand of hypermutated loci. The motif analysis shows that hypermutation occurs predominantly in the coding strand at the first cytosine of CpY elements. FIG. 8B is a graph depicting HM Score and mutation load. HM logo and non-HM logo were separately calculated based on all substitutions from HM and non-HM samples. Given this, HM score of each sample was defined based on its mutation pattern. If mutations in a sample follow the pattern of HM logo, the sample will have higher HM score. Patients with less than ten mutations in either initial or recurrent samples were not considered in the analysis of FIGS. 8B and 8C. By contrast, a pattern of mutations at CpR elements can be seen in all other tumor types tested. Although no significant association in ratios of silent/missense mutations in non-hypermutated tumors was found, recurrent hypermutated samples contained significantly greater numbers of silent mutations. FIG. 8C is a graph providing silent/missense ratio analysis. P-value was calculated by Ranksum test. Moreover, hypermutation can be related to expression of genes involved in tumor recurrence: In hypermutated tumors, those genes containing hypermutated loci are more highly expressed than are mutated genes with no hypermutated loci and non-mutated genes. FIG. 8D is a graph providing expression comparison between three gene clusters: HM genes, mutated (M) genes, and non-mutated (NM) genes. Mean expression of three gene clusters in samples with expression data available (m=160) was calculated to generate the box plot. P-values were calculated by Ranksum test.

### Clonal Evolution and Reconstruction of the Main Routes of GBM Evolution

The number of mutations exclusive to untreated tumors, recurrent tumors, or those in common can be used to describe an evolutionary tree. A method was developed to illustrate and perform statistics on the space of evolutionary trees, termed “moduli spaces” by embedding normalized information of all patients within a sphere. FIG. 9A is a graph providing moduli space of GBM evolution trees. Somatic SNVs or small INDELs with at least 5% allele frequency were considered in this analysis. Each ball represents one patient, and different patterns represent three clusters in moduli space. Here, the upper corner (north pole) represents the fraction of mutations that are common to both samples; the left corner (west) represents the fraction exclusive to the untreated sample, and the right corner (east) the fraction exclusive to recurrence. Unsupervised clustering of the different phylogenies identifies three types of clonal evolution. The lower left group contains many mutations in untreated tumors that are lost in the recurrence, whereas the lower right group is highly enriched with hypermutated recurrences. The top group extends from the north pole to midway along the eastern border, consisting of patients for whom the recurrence includes most of the mutations gained prior to diagnosis as well as additional mutations that could have occurred or become prominent following treatment. The portion of the top group lying along the eastern border represents the limiting case where few mutations are lost from diagnosis, similar to the classical model of linear tumor evolution typified by previous treatment-naïve studies of colon cancer. Treatment however can change linear patterns. The highly branched evolutionary profiles of patients in the central region, far from the eastern border, do not fit this classical model; in these cases, the clones dominant prior to treatment appear to be replaced by new clones sharing few of the same mutations. The possibility of branched evolution makes it difficult to predict the characteristics of the recurrent disease from analysis of the untreated tumor alone.

If many mutations in the initial sample are lost at recurrence, this suggests that the clone dominant at recurrence originated (i.e., diverged from the clone dominant at diagnosis) relatively long before the initial sample was taken. Consistent with epidemiological observations and classical models of tumor evolution of Armitage-Doll and Nordling, the number of mutations in the untreated tumor increases with the patient's age at diagnosis (average of 0.6 protein changing mutations per year or 0.02 per Mb-year). FIG. 10D is a graph providing a correlation between mutation load and age of primary GBM WES-based on TCGA samples. Encouraged by this concordance, a mathematical model of branching tumor evolution was developed to analyze the time at which the recurrence clone originated.

FIG. 9B is a graph providing a model of branching tumor evolution. Initial and recurrent tumors share an ancestral clonal lineage (duration t_{S}), after which they branch off from one another (durations t_{I} and t_{R1}+t_{R2}). After this clonal evolution, the lineage leading to each sample diversifies for a duration t_{MRCA}, during which subclonal variants can accrue. Somatic variants accrue according to substitution rates u₁ and u₂ before and after treatment, respectively. In the branching model, if a mutation occurs along the lineage common to both the initial and recurrent samples, it will be clonal in both of these samples. If a mutation occurs along the lineage leading to the most recent common ancestor (MRCA) of a single sample, then it will be clonal in that sample and absent in the other. If a mutation occurs in a descendant of the MRCA of a sample, then it will be subclonal in that sample and absent in the other. Any other pattern—a mutation that appears subclonally in both samples, or one that appears clonally in one sample and subclonally in the other, would require either recurrent mutation or “back-mutation”. An alternate model, in which the recurrence stems from a lineage nested within the initial sample, can be considered, perhaps selected by therapy. In this case, a single mutational event could produce a variant that is present subclonally at diagnosis and clonally at recurrence, but two events would be needed to explain loss of a clonal mutation. FIG. 10A is a graph illustrating the branching model used in Example 1 of the present disclosure. FIG. 10B is a graph showing an alternative model in which a lineage descended from the most recent common ancestor (MRCA) of the initial sample is selected by therapy to be a progenitor of the recurrent sample (“selection model”). Shaded grey region represents tumor growth, branches represent ancestry of individual cells sampled at the two time points, and light grey loops are drawn around the cells sampled. Though bulk sequencing data was used, this diagram shows individual cellular lineages to illustrate possible relationships. The two models differ in the mutational patterns that can be achieved with a single mutational event. Specifically, only the branching model allows a mutation to be clonal in the initial sample and absent from the recurrence, while only the selection model allows a mutation to be subclonal in the initial sample and clonal in the recurrence. It was found that 59% of patients (54/92) have at least four clonal mutations at diagnosis that are lost in the recurrence, supporting the branching model as the typical scenario. The picture is nuanced, however, as 17 patients have at least four subclonal mutations at diagnosis that become clonal at recurrence, supporting the alternate model as a minority scenario. FIG. 10C is a graph providing a scatterplot/histogram of mutational patterns that distinguish the two models. Each point represents a single patient (shaded points: hypermutated relapse; hollow points: non-hypermutated relapse). The x-axis gives the number of mutations that follow a single-event pattern unique to the selection model, and the y-axis gives the number that follow a single-event pattern unique to the branching model. Most points lie along the y-axis, indicating that the branching model provides a better overall explanation of the cohort.

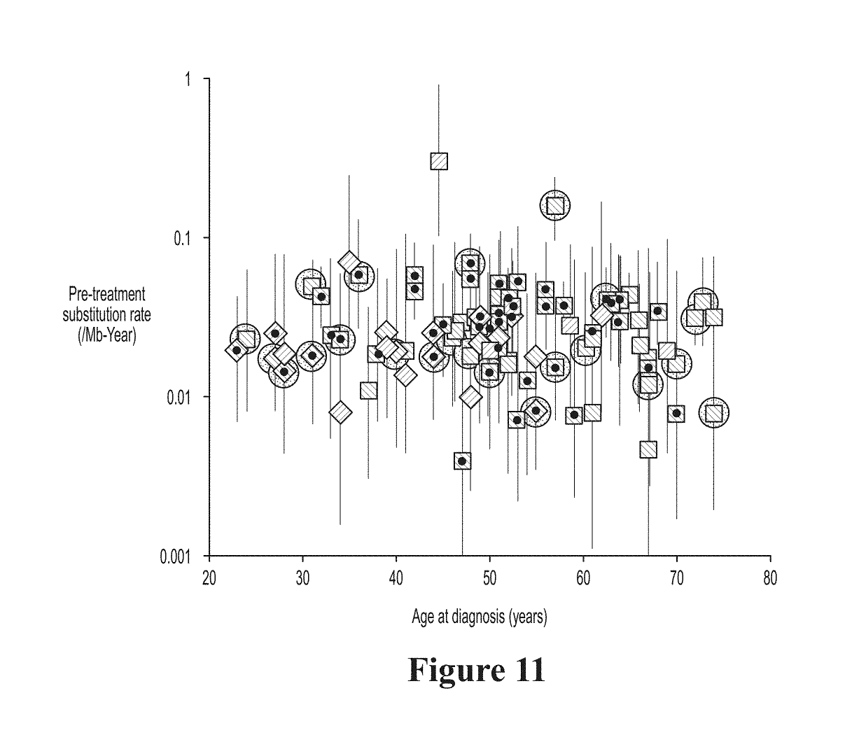

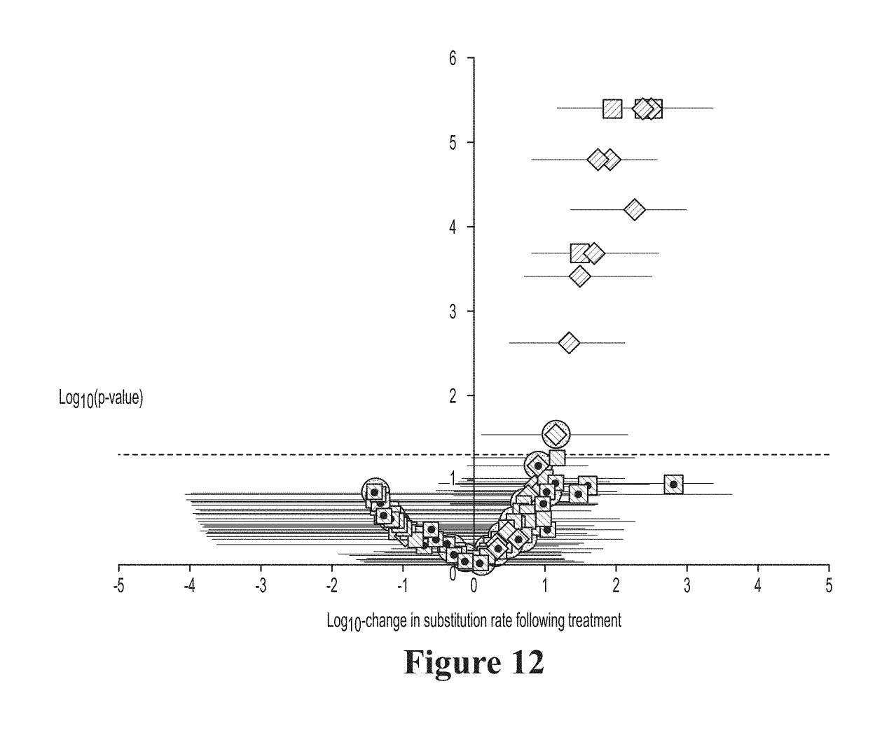

By accounting for the likelihood of each mutational pattern within the branching model, substitution rates before and after treatment, as well as the amount of time before diagnosis that the untreated and recurrence lineages diverged were fit. Using a collection of statistical criteria, it was found that 49% of patients analyzed (45/92) fit well to the model, without requiring an unrealistic frequency of recurrent mutation or “back-mutation”. The pre-treatment substitution rate was consistent among these 45 well-fitting patients, having a median and interquartile range of 0.028 subs Mb-1 yr-1 and 0.018-0.041 subs Mb-1 yr-1. FIG. 9C is a graph showing the relationship between estimated substitution rates before and after treatment, in substitutions per Mb-yr (median and interquartile range for each patient). Dashed line shows diagonal (pre- and post-substitution rates equal). Hypermutated tumors shown in forward slashes, non-hypermutated tumors in backward slashes. Primary GBM diagnoses shown as squares, secondary GBM diagnoses as diamonds. Black dot in the center of symbol shows patients who fit the model well. Circle shows patients with TP53 mutated in both the initial and recurrent samples. Patient R069 was not considered for evolutionary analysis as no valid mutations were detected in the initial sample. Statistics for all 92 patients were similar, with a median (IQR) of 0.024 (0.018-0.035) subs Mb-1 yr-1. No relationship was observed between the substitution rate and age of diagnosis. FIG. 11 is a graph showing that no relationship is evident between pre-treatment substitution rate and age at diagnosis (median and 95% credible interval shown for each patient). The symbol legend is the same as described in connection with FIGS. 9C and 9D. Considerably more variation was observed in post-treatment substitution rates, with 15 of the 92 patients exhibiting significantly higher mutation rates after treatment. FIG. 12 is a graph depicting a volcano plot of fold-change in substitution rate following treatment (median and 95% credible interval of ratio of post- to pre-treatment substitution rates). Horizontal dashed line shows p=0.05. The symbol legend is the same as described in connection with FIGS. 9C and 9D. All but one of these patients showed hypermutation, with over 500 variants found in the recurrent sample.

Estimates of divergence time suggest that the recurrent clone diverged from the untreated clone many years before disease was detected. FIG. 10E is a graph showing the distribution of estimated time before diagnosis when the initial and recurrent lineages diverged, in years (median and 95% credible interval for each patient). The median among the 45 well-fitting patients had a divergence time of 12.6 years (range 2.3-50.5, IQR 7.2-22.6). Since the remaining 47 non-fitting patients can fail the model's assumption that untreated and recurrent tumors be evolutionarily distinct (i.e., monophyletic), divergence time for these patients be interpreted only as a heuristic measure of genetic difference between the two tumor samples. In general, uncertainty in the divergence time was large, with the median well-fitting patient showing a 95% CI 24 years wide. Still, even the bottom of the 95% CI exceeded three years for a majority of patients.

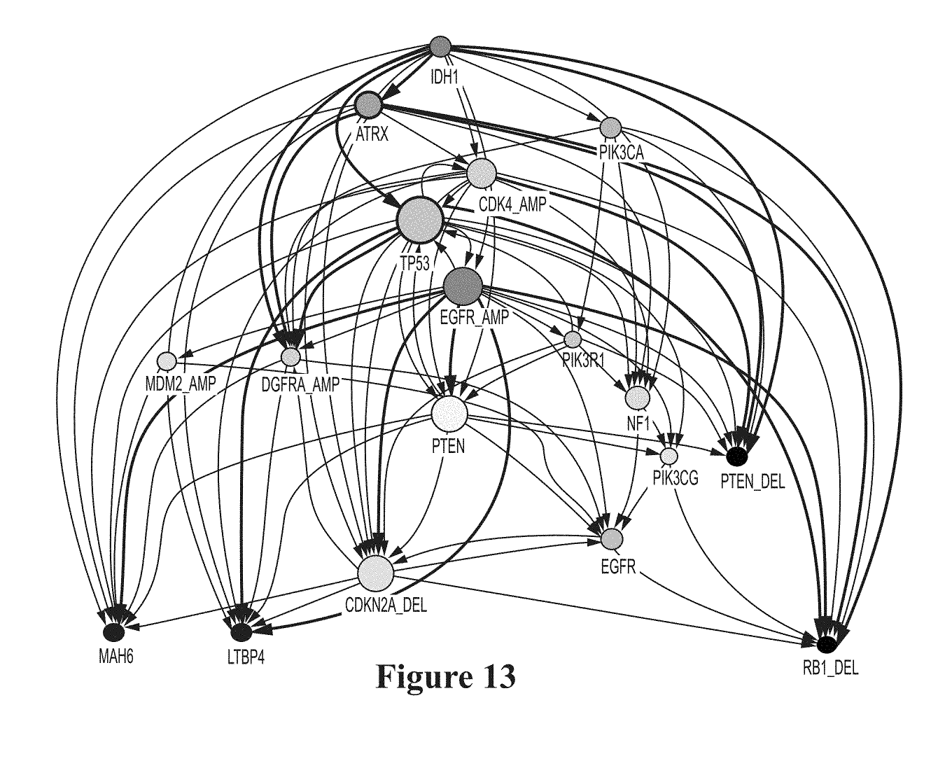

To reveal the potential evolutionary trajectories of GBM under therapy a tumor evolutionary directed graph (TEDG) was constructed for the 93 triplet samples. As this analysis uses as input the fraction of cells harboring a particular mutation, the purity of the tumor was estimated using ABSOLUTE and PyClone. The resulting TEDG indicates that mutations in IDH1, PIK3CA and ATRX are early events, mutations in TP53, NF1, and PTEN occur later, and mutations in MSH6 and LTBP4 are relapse-specific events. FIG. 9D is a graph showing cross-sectional integration of longitudinal data by tumor evolutionary directed graph. Arrow represents the time order of mutations. Thicker arrows represent that there are more independent patients containing the same order of mutation. The size of the node indicates the frequency of the mutations in the cohort. A more complex set of possible evolutionary trajectories appears when copy number information is included in the analysis. FIG. 13 is a graph providing tumor evolutionary directed graph of both gene mutations and copy number alterations. The symbol legend is the same as described in connection with FIG. 9D.

### Clonal Replacement Events are Common Throughout GBM Evolution

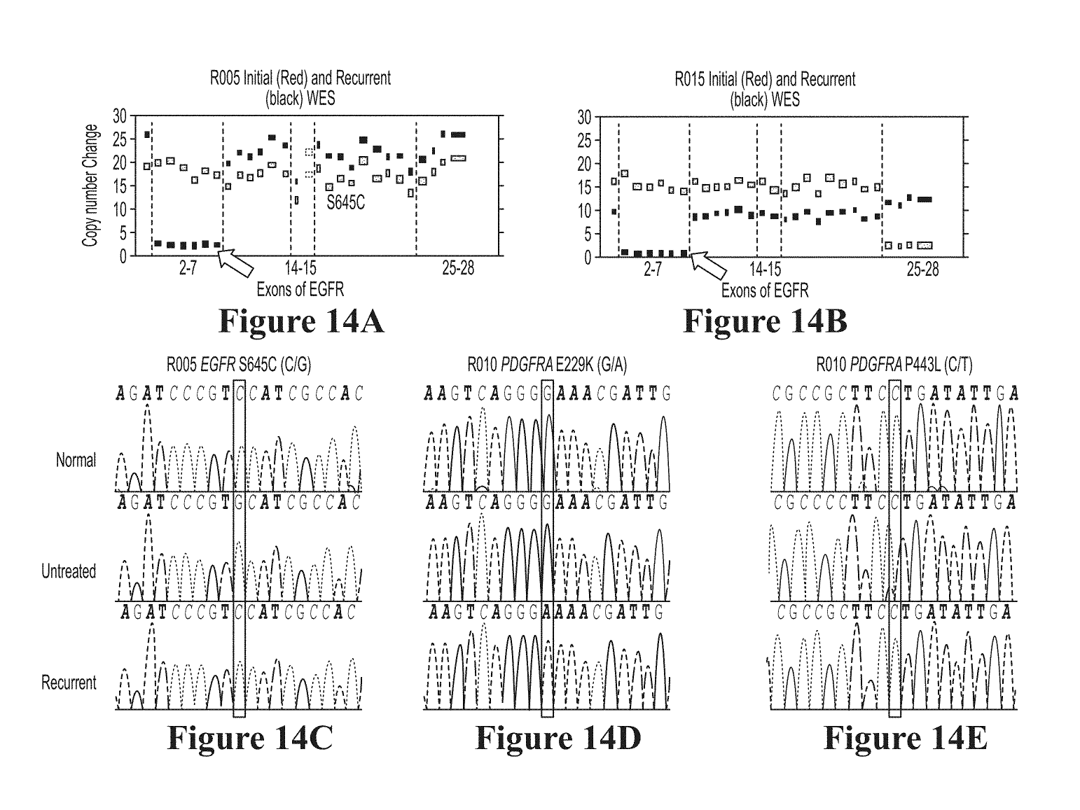

To discover the pattern of alterations in recurrent GBM compared with untreated tumors, in-depth investigations into any gains or losses of genetic alterations were performed. The epidermal growth factor receptor (EGFR) gene is known to be frequently amplified, mutated, and rearranged in untreated gliomas. To uncover the role of EGFR alterations in GBM evolution, PRADA was applied to detect EGFR structure variance from RNA sequencing data. By calculating junction reads at least one junction read of EGFRvIII was found in 18% (12/67) of initial tumors and 11% (8/76) of recurrent tumors. Interestingly, nine patients lost EGFRvIII and one patient gained EGFRvIII at relapse (transcribed allelic fractions>5%), indicating, first, that EGFRvIII is a late event originated after the clonal lineages leading to the two samples diverged and, second, that EGFRvIII is more common in initial tumors and lost during treatment (FIG. 1D). An example is patient R005 whose untreated tumor harbors EGFR amplification and the S645C mutation. The EGFR S645C mutation was lost in the recurrent tumor and replaced by EGFRvIII. FIGS. 14A-14E provide evidence of clonal switching of key driver genes during GBM evolution. FIGS. 14A-14B are two graphs showing rearrangement of EGFR exons based on WES data. The x-axis indicates 28 exons of EGFR gene, and the y-axis indicates the copy number change compared with corresponding normal control. Shaded dots indicate untreated samples, while black dots indicate recurrent samples. Gain of EGFRvIII was highlighted. FIGS. 14C-14E are graphs providing Sanger validation of mutations of some key driver genes.

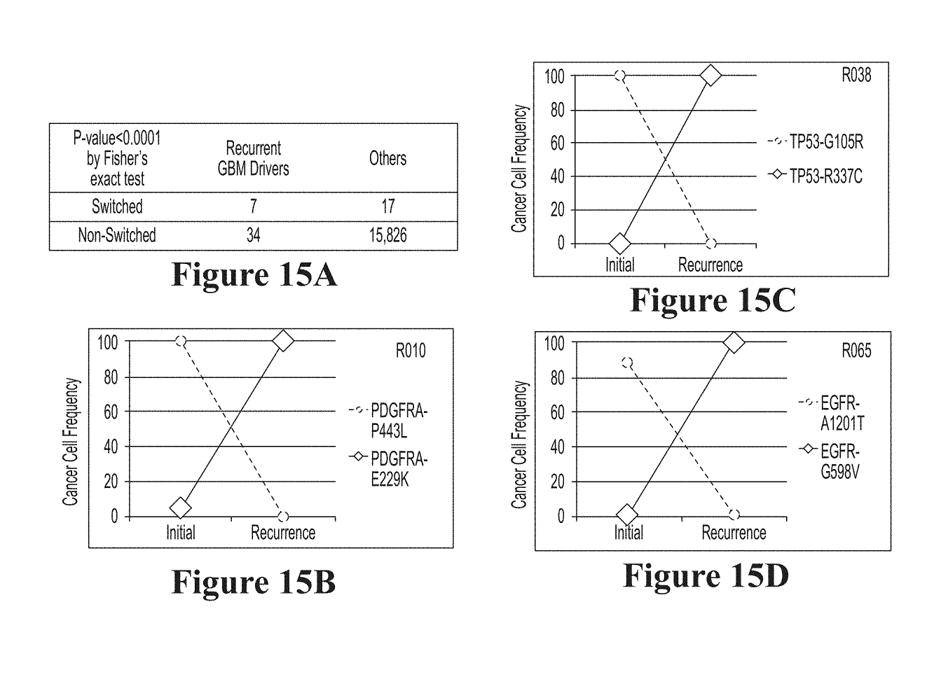

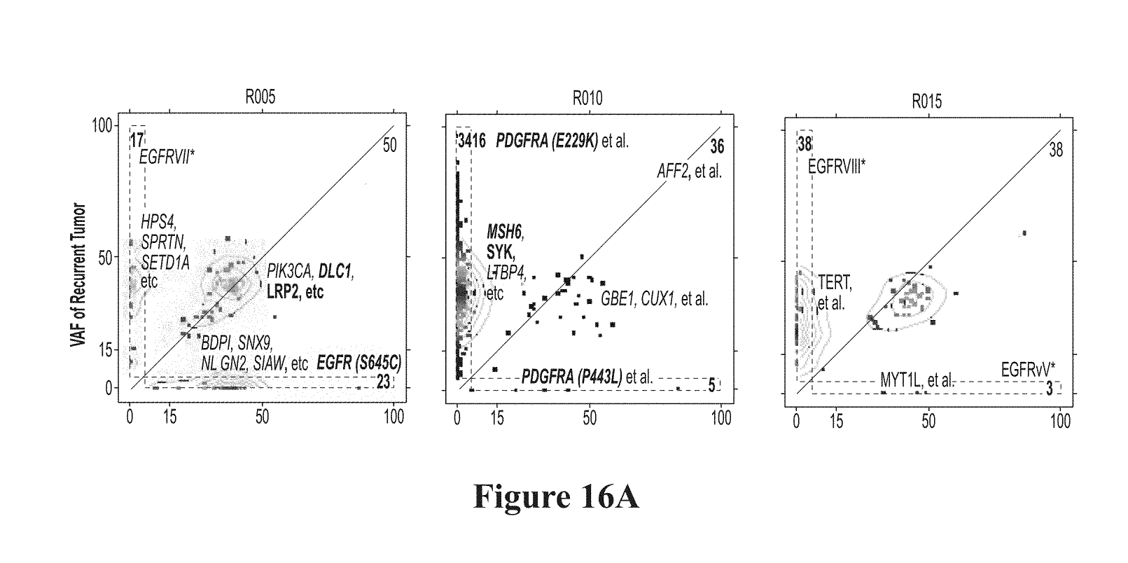

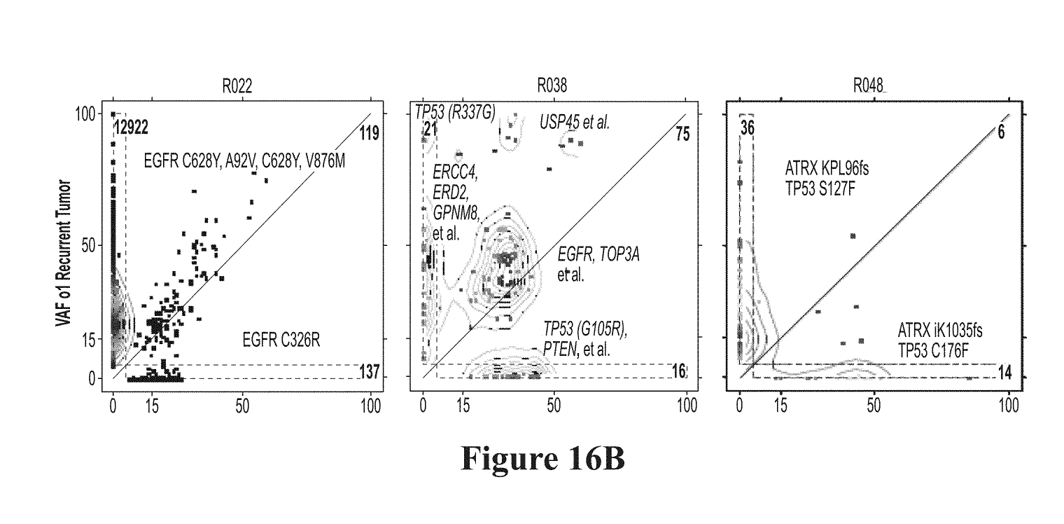

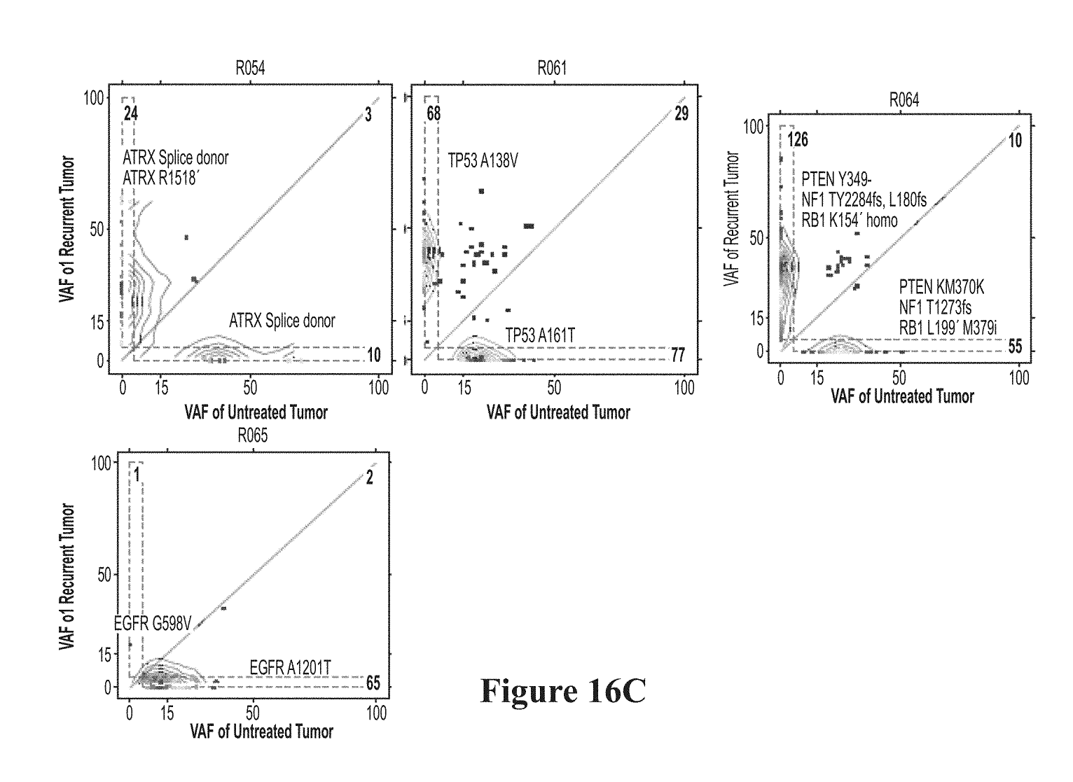

A switch between differently mutated versions of the same gene also occurs in platelet-derived growth factor receptor alpha polypeptide (PDGFRA), another RTK-coding gene frequently activated in GBM (FIG. 14). FIG. 15 show clonal replacement in key driver genes. Cancer cell frequency was estimated by Pyclone. FIG. 15B is a graph showing PDGFRA mutational replacement in one patient. Mutation E229K, which is relatively common in cross-sectional mutation databases (e.g., TCGA), appears to be a relatively late event, as it is exclusive to recurrence and replaces the initial mutation P443L. Mutational replacement also occurs in the tumor suppressor TP53 (G105R to R337C in Patient R038, FIG. 15C is a graph showing TP53 mutational replacement in the patient) and in EGFR (A1201T to G598V in Patient R065, FIG. 15D is a graph showing EGFR mutational replacement in the patient). In all, 11% (10 out of 93) of recurrent GBM patients have clonal replacements within key drivers. FIGS. 16A-16C are graphs providing mutation contour plots, illustrating the variant allele fraction (VAF) of somatic SNVs and small INDELs in untreated and recurrent tumors of patient with clonal switching in key driver genes. The gray dashed rectangles indicate mutations undetectable (<5%) in one of those two samples. The numbers inside the square indicate the number of mutations in common, only in recurrent, and only in untreated tumor. Mutations in bold were validated by Sanger, and those in italicized texts were expressed. The dot shades and the contours indicate node density. These clonal switching events within the same gene occur preferentially in genes known to play a role in GBM (p-value<10⁻⁴). FIG. 15A is a table showing that mutations of seven key GBM drivers (EGFR, TP53, PDGFRA, PTEN, ATRX, NF1, and RB1) were replaced by different mutations in the same genes. The strong association between switching alterations and key driver genes (EGFR, TP53, PDGFRA) suggests (1) some of these genes contribute to a late expansion both with treated and untreated tumors and (2) converging evolution is associated to these genes.

### Expression Analysis and Subtype Switching

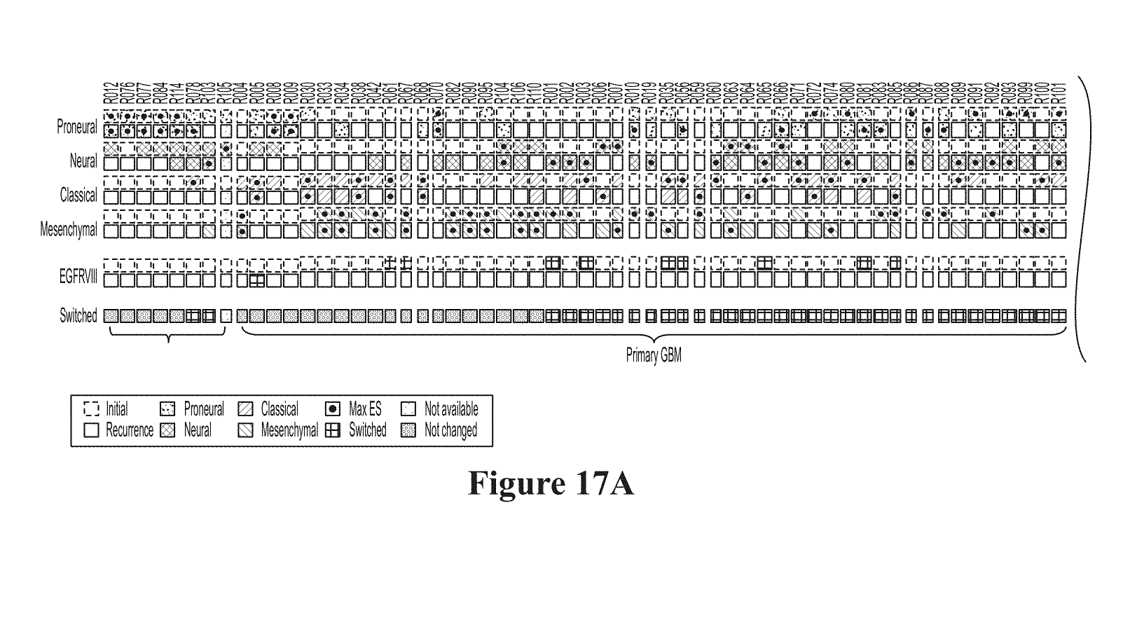

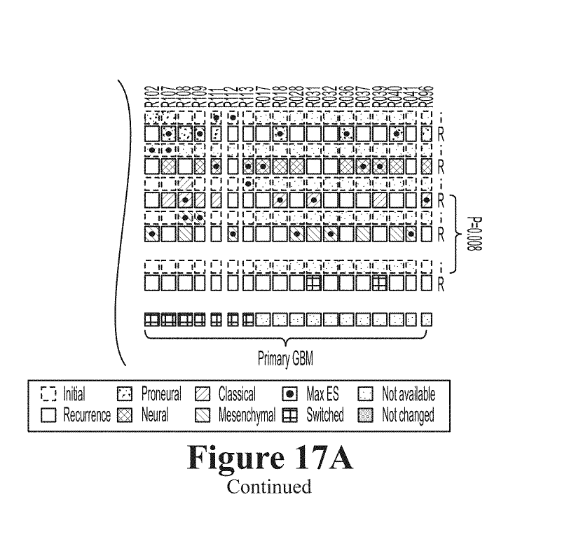

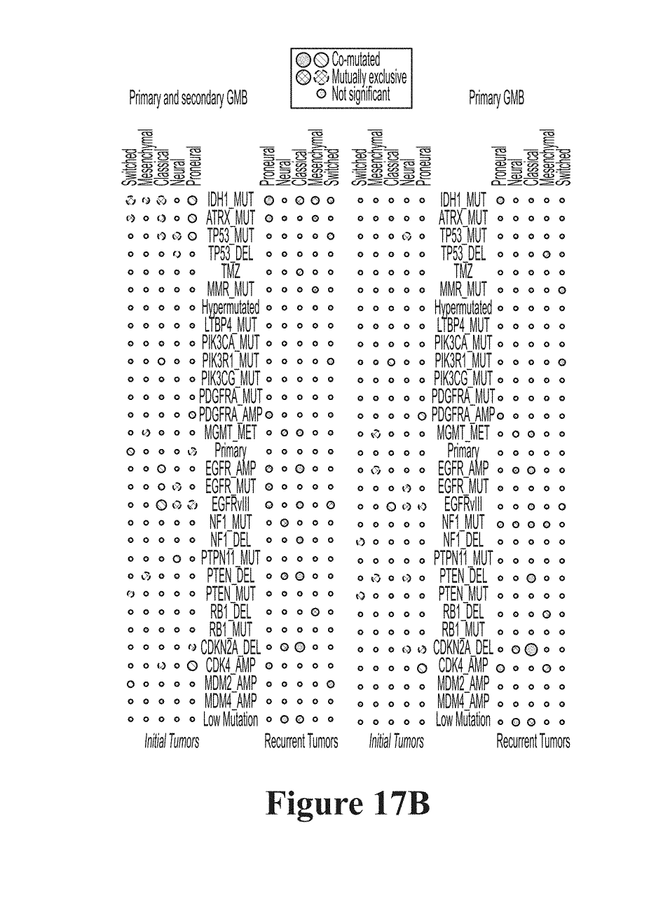

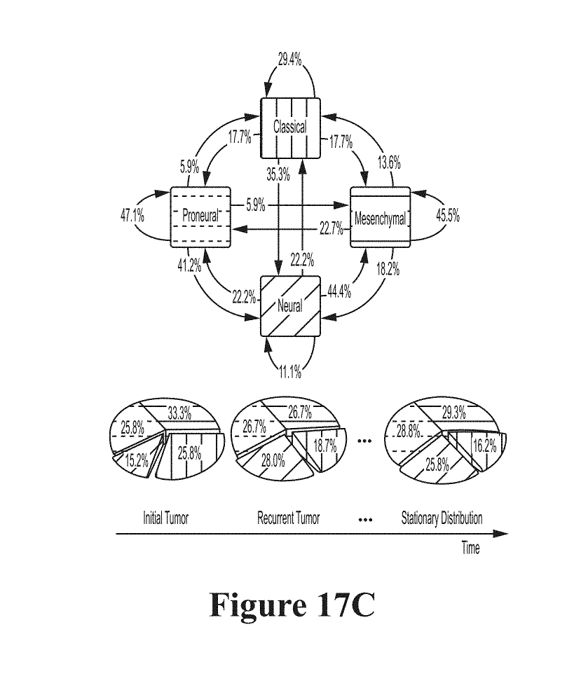

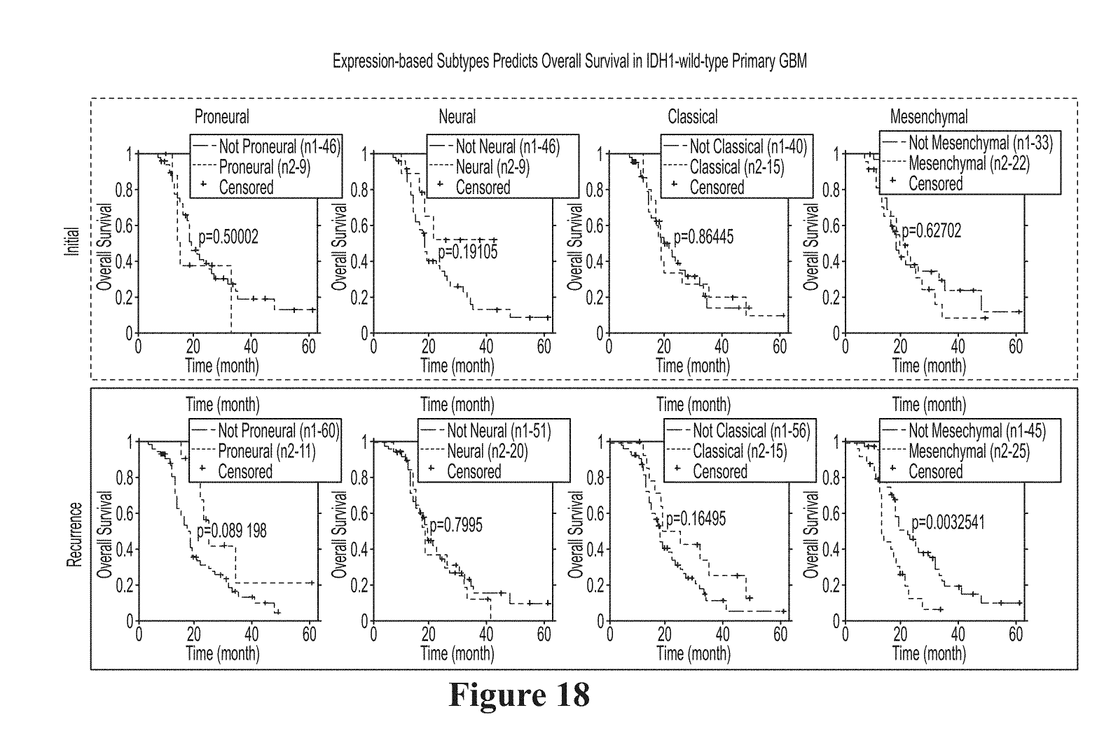

Based on its pattern of gene expression, GBM is commonly divided into four subtypes, which display different responses to treatment. To study evolution of gene expression in GBM, the ssGSEA method was used to subtype each tumor sample. FIG. 17A is a graph depicting expression based GBM subtyping. ssGSEA was performed to cluster each sample into four subtypes (proneural, neural, classical, and mesenchymal). “*” indicates subtypes with maximal enrichment score (ES). If the optimal subtype in initial and that in recurrent tumor is different, a patient was labeled as switched. P-value was calculated by Fisher's exact test. As expected, it was found that IDH1 mutated patients are mostly classified as proneural gliomas; EGFR alterations are associated to classical subtype; and NF1 alterations to mesenchymal subtype. FIG. 17B is a graph illustrating the association between expression-based subtype switching and genetic/clinic features. The same analysis as described in connection with FIG. 1C was performed. It was observed that all five hypermutated primary GBM cases switched their subtypes (two to mesenchymal, one to neural and two to proneural). Strikingly, it was found that two-thirds of primary GBM cases (39/58) switch transcriptional subtype at relapse, while secondary GBM cases are more stable (2/7 switched) (FIG. 17A). Interestingly, mesenchymal subtype is the most stable primary GBM, switching in 55% (12 of 22 primary GBM) of cases at recurrence; and mesenchymal subtype at recurrence is associated with worse overall survival (p-value=3×10⁻³). FIG. 18 is a graph showing that expression-based GBM subtyping predicts GBM overall survival in recurrent tumor. P-values were calculated based on logrank test. Only IDH1 wild type primary GBM was considered in this analysis. As EGFRvIII is associated to the classical subtype (FIG. 17B), loss of this alteration in the recurrent tumor is consistently associated to the transition from classical to other expression subtypes (FIGS. 17A and 17C, p-value=8×10⁻³, Fisher's exact test). FIG. 17C is a graph providing the stochastic matrix of GBM subtypes. The large cohort of longitudinal GBM samples allows the construction of a probability transition matrix between four subtypes. The arrows indicate the frequency of a patient to stay a subtype or to be switched from one subtype to another. A stationary distribution was calculated based on this stochastic matrix, indicating the proportion of these four subtypes after treatment.

### LTBP4 Expression Promotes Tumor Growth and Reduces Survival

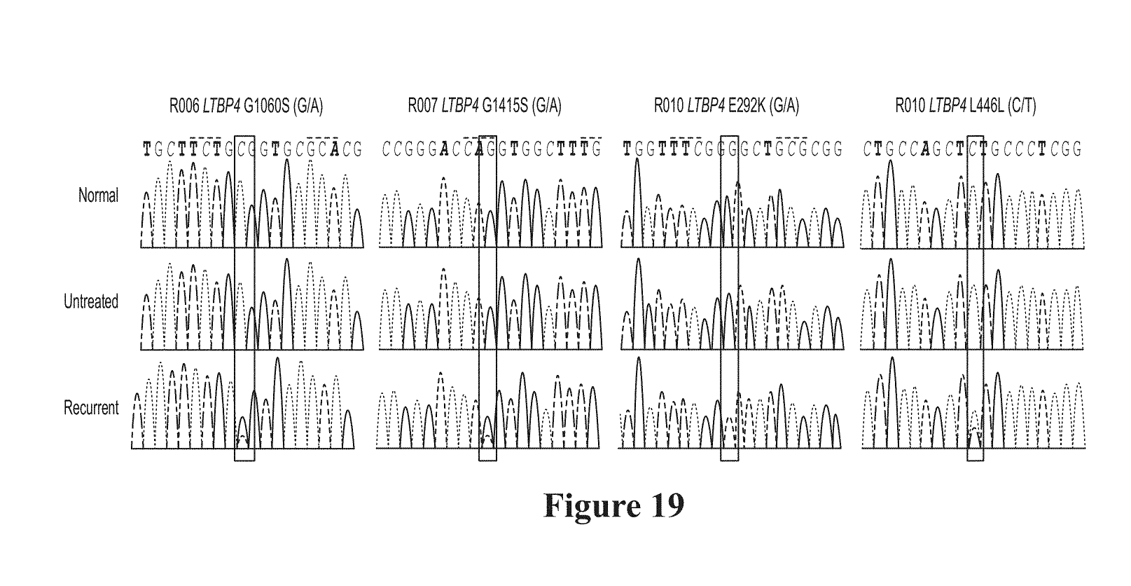

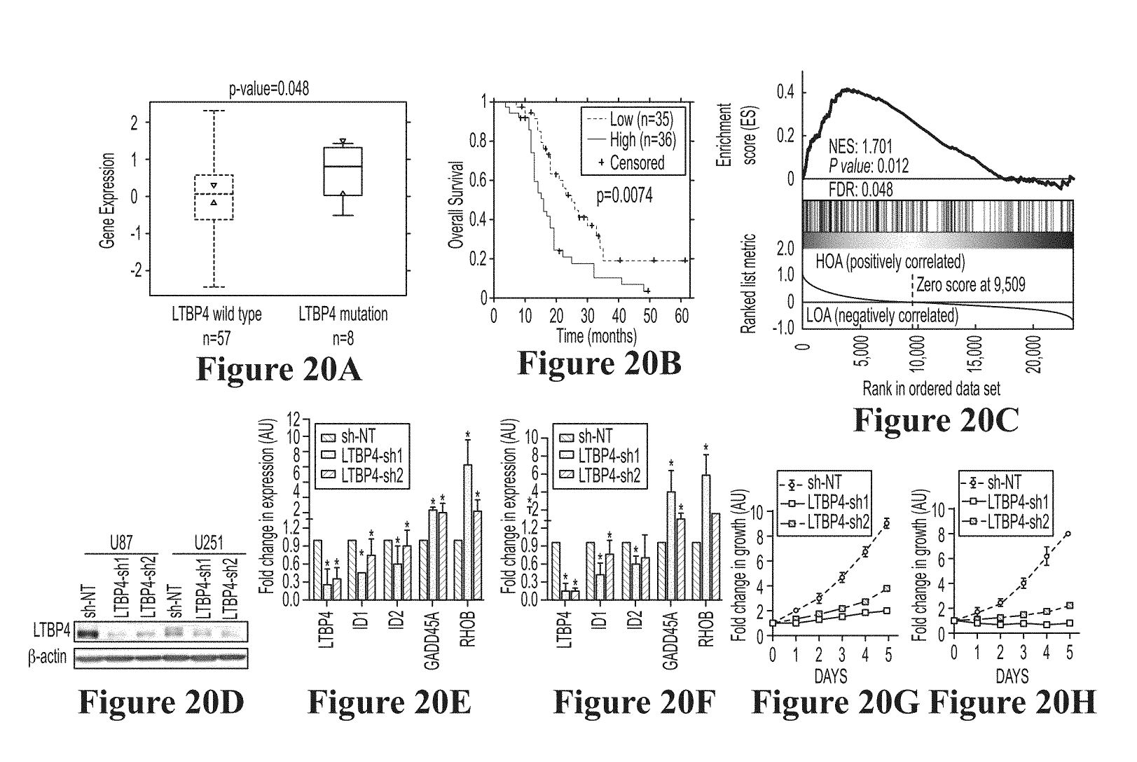

It was found that the gene Latent transforming growth factor beta binding protein 4 (LTBP4) harbors significantly more mutations in recurrent than untreated GBM (FIG. 1D, FIG. 19). FIG. 19 is a graph providing Sanger validation of LTBP4 mutations in cohort INCB. The LTBP4 gene codes for a protein that belongs to the LTBP family, which is implicated in the regulation of the TGF-β pathway, typically acting as activator of TGF-β signaling. Interestingly, activation of TGF-β is known to drive aggressiveness of malignant glioma, and it was found that high expression of LTBP4 in recurrent tumors is associated with worse prognosis in IDH1-wild-type primary GBM patients (p-value=7×10⁻³). FIG. 20B is a graph showing survival analysis of LTBP4 expression in IDH1-wild-type primary GBM patients. High indicates z-score of LTBP4>0, while low are LTBP4<0. P-value was calculated by log rank test. Only IDH1-wild-type primary GBM patients were considered in this analysis. Furthermore, mutations of LTBP4 are correlated with higher expression of this gene (p-value<0.05). FIG. 20A is a graph showing that LTBP4 mutation was related to its high expression in recurrent GBM. P-value was calculated by Ranksum test. Further strengthening the case that LTBP4 expression could drive tumor growth via TGF-β activation, elevated expression of LTBP4 in GBM is associated with elevated expression of genes implicated in the TGF-β pathway (Gene Set Enrichment Analysis FDR<0.05). FIG. 20C is a graph providing gene set enrichment analysis. Recurrent tumor samples from IDH1-wild-type primary GBM were grouped according to LTBP4 expression. Samples with high LTBP4 expression (z score>0) were enriched with TGF-β activity.

To experimentally validate the functional link between LTBP4 and TGF-β, lentiviruses carrying two independent LTBP4 shRNA cassettes were used to silence the LTBP4 gene in the human glioma cell lines U87 and U251. FIG. 20D is an image depicting knocking down of LTBP4 in two cell lines. Two independent shRNA cassettes were used. LTBP4 silencing in both cell lines resulted in reduced expression of the ID genes ID1 and ID2, which are positively regulated by TGF-β in glioma. Conversely, LTBP4 silencing also led to the up-regulation of RhoB and GADD45a, two genes repressed by TGF-β in glioma. FIGS. 20E-20F are two graphs showing gene expression changes in U87 (FIG. 20E) and U251 (FIG. 20F). Consistent with the pro-tumorigenic role of TGF-β in GBM, LTBP4 silencing markedly impaired proliferation of U87 and U251 glioma cells. FIGS. 20G-20H are two graphs showing cell proliferation of U87 (FIG. 20G) and U251 (FIG. 20H).

### Discussion

Using longitudinal genomic and transcriptomic analysis of 114 GBM patients, the major routes of GBM evolution under therapy were detailed. GBM evolution is highly branched, and specific alterations and evolutionary patterns are associated with treatment. Despite 45% of mutations (in non-hypermutated tumors) being shared between diagnosis and relapse samples, the dominant clone at diagnosis is generally not a lineal ancestor of the dominant clone at relapse. Instead, these two clones diverged from a common ancestor more than a decade before diagnosis in most patients (FIG. 10E).

Since 11% of patients (10/93) exhibit replacement of one mutated version of a gene (at diagnosis) with another, differently mutated version of the same gene (at relapse), it is conceivable that genes associated with undergoing clonal completion are late driver events. In fact, this mutational switching phenomenon is enriched ˜200-fold in genes known to be implicated in GBM, including EGFR, TP53, and PDGFRA (FIG. 15A, FIG. 18). This scenario of convergent evolution suggests that the common ancestor of diagnosis and relapse clones had fewer driver alterations and therefore a less aggressive phenotype. The accumulation of alterations in GBM cells therefore seems to occur over a decade(s)-long growing phase that leads to a highly diverse population, each clone experiencing a parallel series of expansions.

Related to mutational switching, it was found that two-thirds of primary GBM patients exhibit different transcriptional subtypes at diagnosis and relapse. This observation of subtype switching, considered together with recent findings that different parts of the same tumor can exhibit different GBM subtypes, also calls into question the significance of the expression-based classification as a prognostic marker prior to relapse.

Evolutionary dynamics generally appear similar before and after treatment: the mathematical model estimates typical substitution rates of ˜0.03 substitutions per Mb per year during both periods, except in the 16% of cases that recur with hypermutated tumors. Hypermutated tumors, which are highly enriched for mutations at CpC dinucleotides, harbor mutations in mismatch repair (MMR) genes, most commonly in MSH6, and can exhibit 100-fold higher substitution rates (˜3 substitutions per Mb per year). It was found that hypermutation preferentially targets highly expressed genes, suggesting that the mutagenic mechanisms related to TMZ treatment and subsequent MMR alteration act more efficiently in highly expressed regions of open chromatin.

Finally, and of particular relevance to discovery of novel GBM treatment, unique alterations associated with relapsed GBM were uncovered. In addition to previously reported mutations in MMR genes in 15% of patients (14/93), mutations in the LTBP4 gene were found in 11% of relapsed tumors (10/93). LTBP4 encodes a protein that binds to transforming growth factor beta (TGF-β). The TGF-β signaling pathway has been associated in a variety of biological contexts including proliferation, epithelial to mesenchymal transition, and apoptosis. Both clinical and in vitro evidence exists that LTBP4 activates this signaling pathway to drive tumor growth: Higher expression of LTBP4 in IDH1 wild-type primary GBM associated to poorer survival (FIG. 20B, p-value=7×10⁻³), and silencing LTBP4 in two different cell lines decreases both proliferation and activity of TGF-β target genes. These results are consistent with recent animal studies showing that TGF-β inhibitors reduce viability and invasion of gliomas and advance the case for these molecules as potential anti-tumor therapeutics.

In conclusion, this study sketches the main routes of GBM evolution under therapy, identifying a highly branched process with specific alterations and evolutionary patterns associated to treated tumors.

### Example 2: Spatiotemporal Genomic Architecture Informs Precision Oncology in Glioblastoma

This Example provides methods of identifying mutations in PAM pathway in patients with GBM. Precision medicine in cancer proposes that genomic characterization of tumors can inform personalized targeted therapies. However, this proposition is complicated by spatial and temporal heterogeneity. Here, genomic and expression profiles across 127 multisector or longitudinal specimens from 52 individuals with glioblastoma (GBM) are studied. Using bulk and single-cell data, it was found that samples from the same tumor mass share genomic and expression signatures, whereas geographically separated, multifocal tumors and/or long-term recurrent tumors are seeded from different clones. Chemical screening of patient-derived glioma cells (PDCs) shows that therapeutic response is associated with genetic similarity, and multifocal tumors that are enriched with PIK3CA mutations have a heterogeneous drug-response pattern. It is shown that targeting truncal events is more efficacious than targeting private events in reducing the tumor burden. In summary, this Example demonstrates that evolutionary inference from integrated genomic analysis in multisector biopsies can inform targeted therapeutic interventions for patients with GBM.

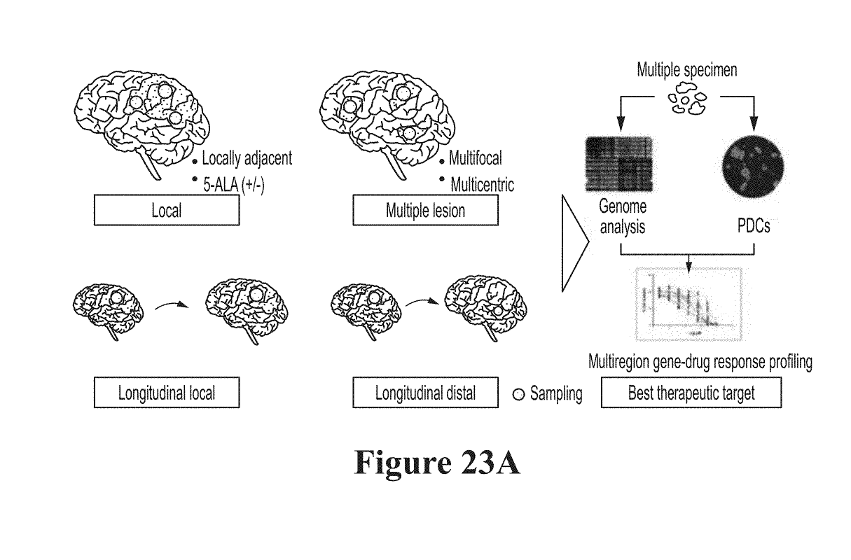

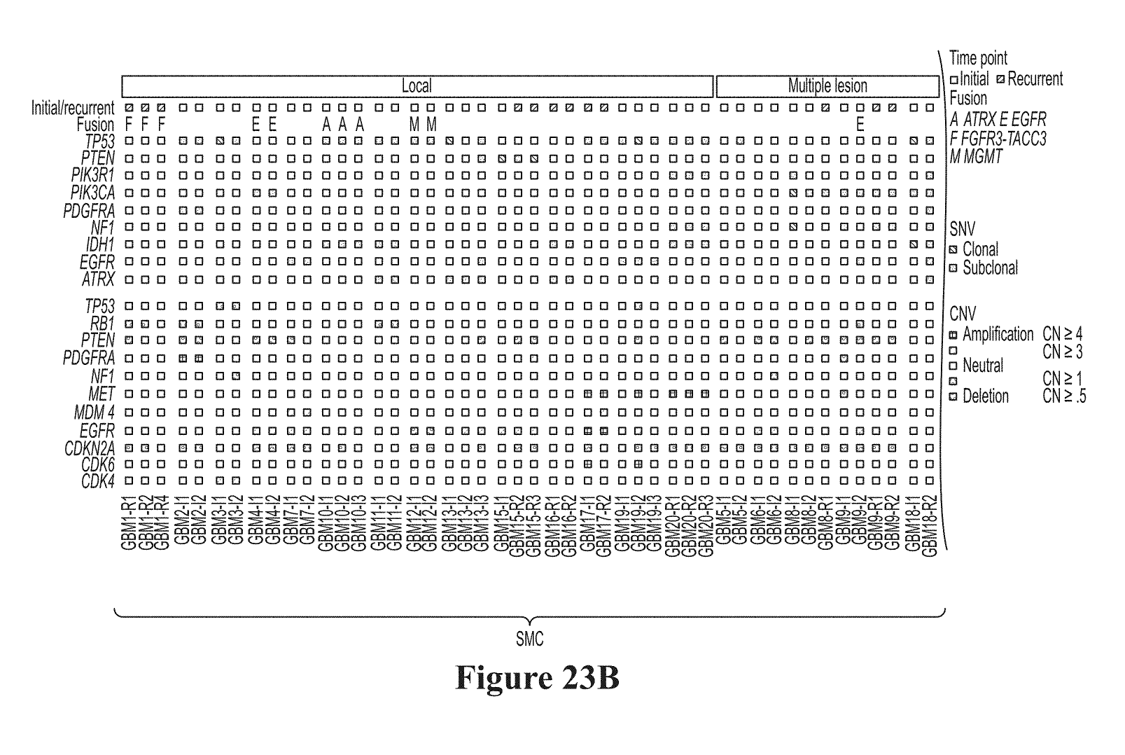

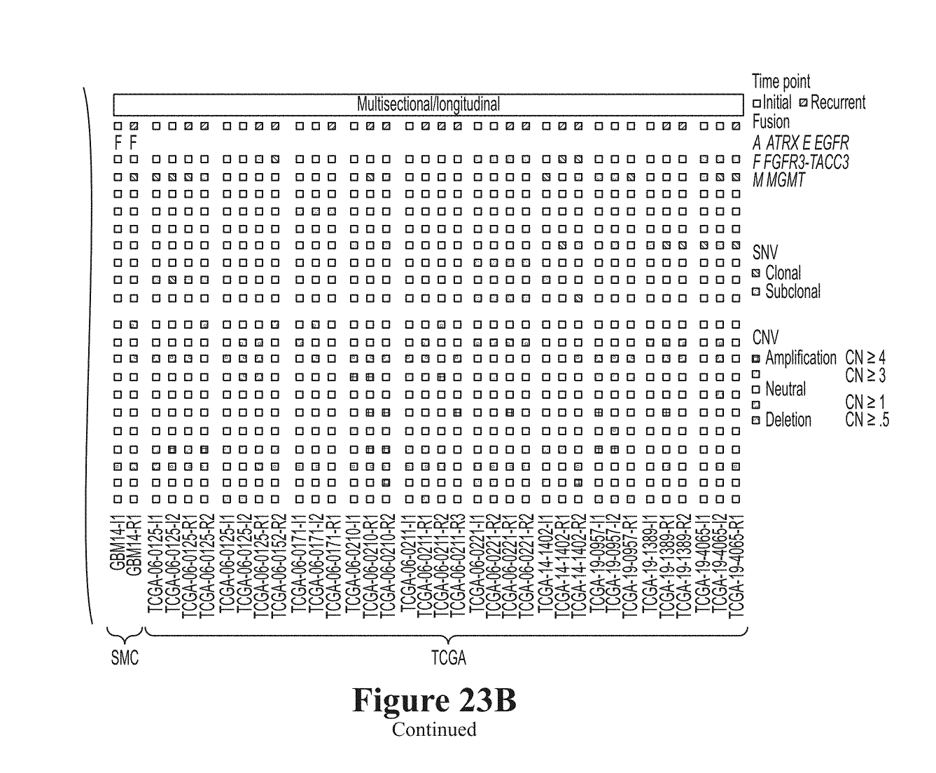

Most clinical trials for targeted therapy in GBM have shown limited clinical success. Although recent genome-wide studies evaluating regional heterogeneity and longitudinal GBM pairs have suggested potential evolutionary models of the tumors, there is little understanding regarding which strategies can effectively use genomic data to inform targeted therapies. To identify such strategies, somatic variants in 127 multiregion or longitudinal tumor specimens from 52 individuals with glioma were analyzed: 42 individuals from the Samsung Medical Center (SMC), Seoul, and 10 individuals from The Cancer Genome Atlas (TCGA) GBM cohort. Additionally, the transcriptomes of 83 tumor specimens from 41 individuals (bulk) and 305 single cells from 7 samples for 3 individuals were analyzed. Tumors were classified into four distinct groups according to the spatial and temporal features of tissue acquisition: tumors obtained from the same location at the same time (locally adjacent), tumors obtained from different locations at the same time (multifocal/multicentric; referred to as multiple), and tumors obtained from local and distant recurrences at different times (longitudinal local and distant, respectively). FIG. 23A is a graph showing a schematic representation of glioma genomic heterogeneity and differential drug-response analysis. Human glioma specimens were acquired on the basis of their spatial order or longitudinal pairing and were subjected to genomic analysis for identification of tumor-initiating (truncal) events. PDCs refer to patient-derived cells and 5-ALA refers to 5-aminolevulinic acid.

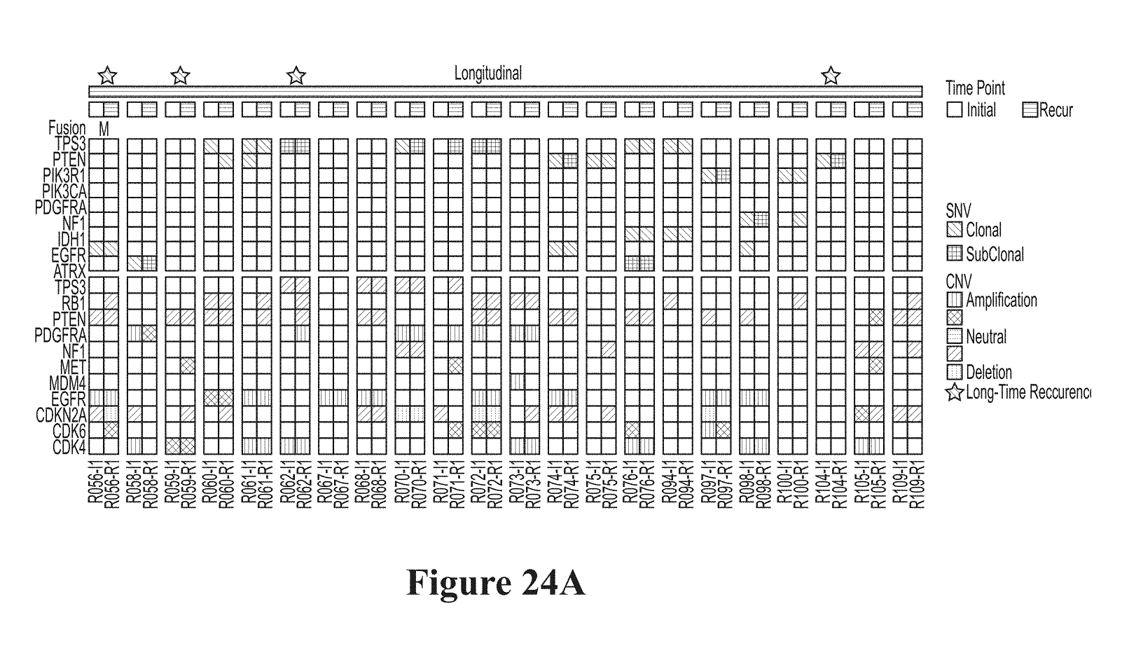

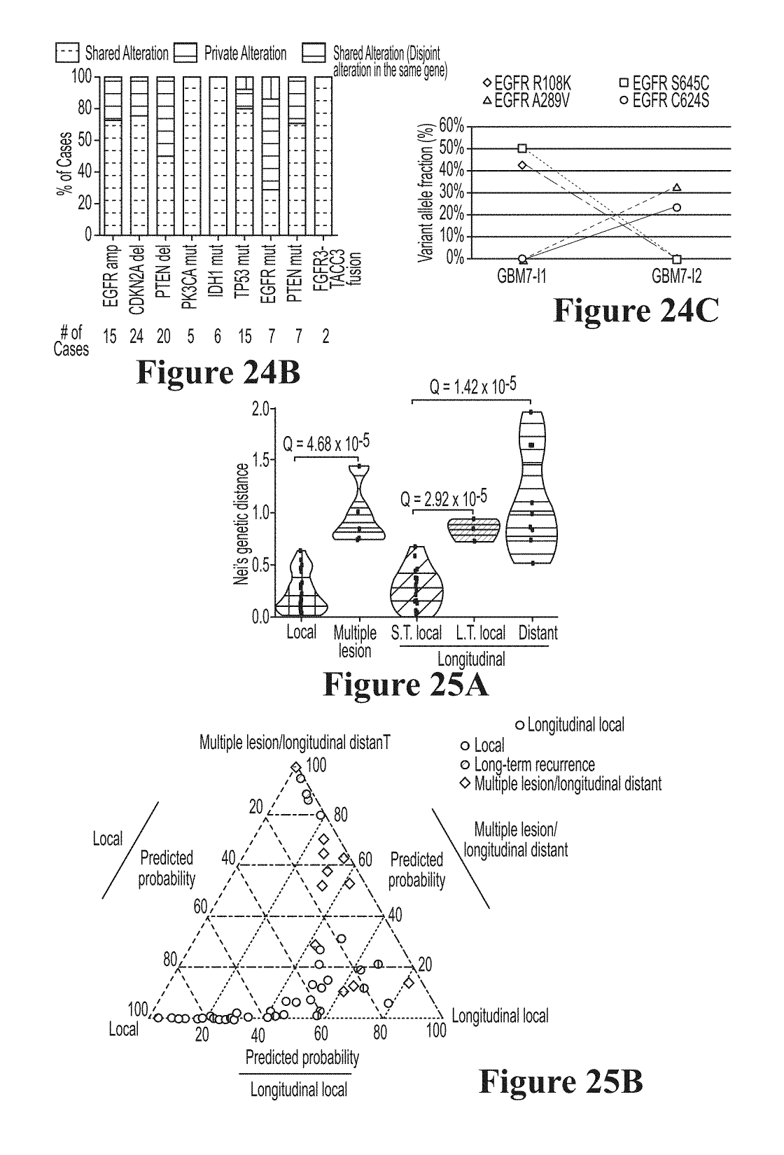

Clonal and subclonal alterations from cancer cell fractions in multiple sectors were inferred (see Methods of “Somatic mutation”, “Copy number” and “Cancer cell fractions and clonality”). The average mutation rate was 2.2 mutations/Mb for nonhypermutated samples, which is consistent with previous studies. IDH1 mutations mapping to Arg132 were clonal across all of the regions in IDH-mutant tumors2, 14 (6/6). PIK3CA mutations were always clonal and shared by all sectors (5/5), which is consistent with the results of our previous longitudinal analysis from tumor evolution directed graphs (TEDGs), in which it was found that PIK3CA mutations are early events. FIG. 23B is a graph providing somatic mutations, including single-nucleotide variants (SNVs) and small insertions/deletions, copy number alterations, and gene fusions, for 83 glioma multiregion or multisector longitudinal specimens from 30 patients. 34 locally adjacent tumor fragments from 14 patients, 13 multifocal/multicentric (referred to as multiple) tissues from 5 patients, and a longitudinal pair, GBM14, with leptomeningeal seeding were collected from the Samsung Medical Center (SMC). 34 multisector longitudinal tumor exomes and/or RNA sequencing data from 10 patients in The Cancer Genome Atlas (TCGA) cohort were curated. All somatic mutations called by SAVI with an allele frequency of >5% are shown. For each gene, copy number (CN) was calculated on the basis of EXCAVATOR results. Clonal alterations were determined using ABSOLUTE with a cancer cell fraction of >80%. CNV refers to copy number variation. FIG. 24A is a graph showing landscape of somatic mutations for 22 pair longitudinal cohort. FIG. 24B is a graph showing shared or private presence of major GBM driver gene alterations in 29 (excludes case GBM14 from FIG. 1B) multisector cases from SMC and TCGA cohort. A shared event in a multi-sectional case is defined such that all primary and/or all recurrent sections have the same mutational event. Furthermore, FGFR3-TACC3 fusions were highly expressed in all regions from two individuals. These somatic variants, which are shared by all tumor regions, represent promising therapeutic targets, as they reflect truncal alterations in the evolutionary tree that are suspected to be present among all tumor cells. In contrast, PTEN alterations, including copy number deletions and mutations, were shared by 10 of 20 (50%) and 5 of 7 (71.4%) tumor sectors, respectively. Likewise, EGFR amplifications were observed as private events, exclusive to only one or two regions of the multisector sample, in 4 of 15 (26.7%) of the EGFR-amplified tumors, including 2 multiple cases (GBM5 and GBM9). Furthermore, EGFR mutations were shared by 3 of 7 (42.9%) cases, including one harboring disjoint alterations, which are different genomic alterations in the same gene (GBM7-I1: p.Leu62Arg and p.Arg108Lys; GBM7-I2: p.Ala289Val and p.Cys624Ser), suggesting that partial genetic information from a single tumor biopsy can be inconclusive in assessing the potential benefit from EGFR-targeted therapy (FIGS. 24B-24C). FIG. 24C is a graph providing disjoint mutation pattern of EGFR in multisector samples. VAFs of 4 different EGFR mutations (S645C, C624S, R108K and A289V) were plotted on parietal or ventricular tumor fragment in GBM7 patient.

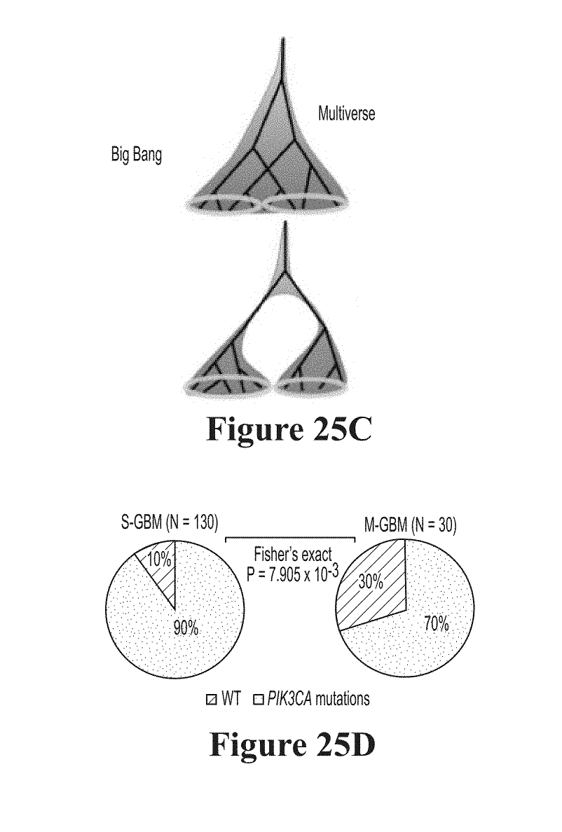

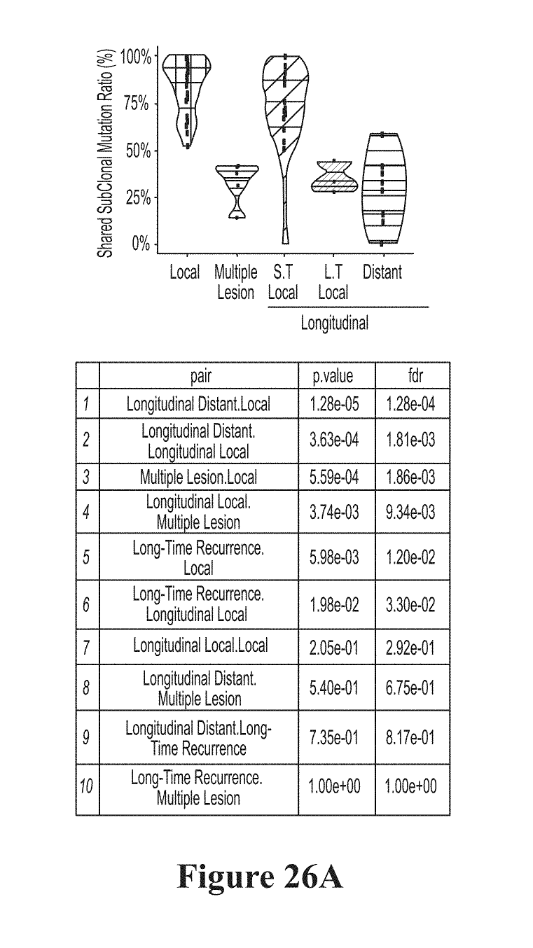

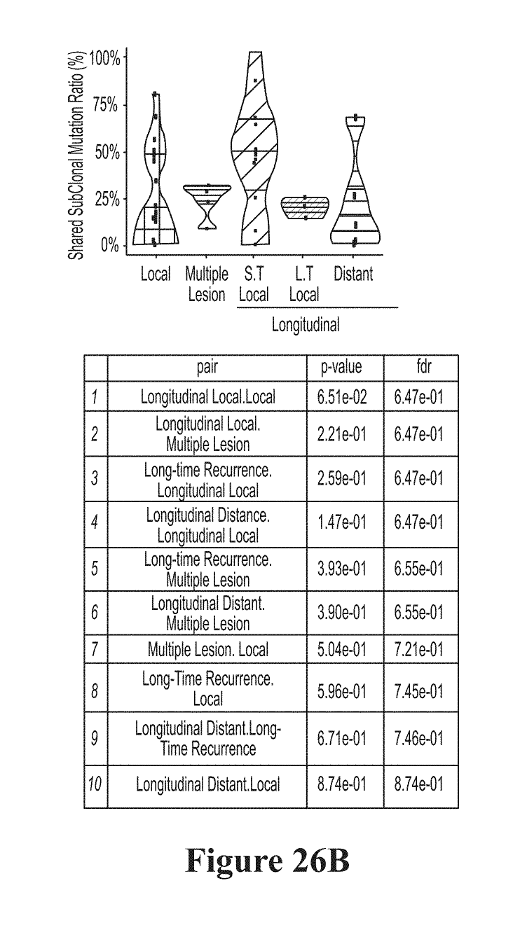

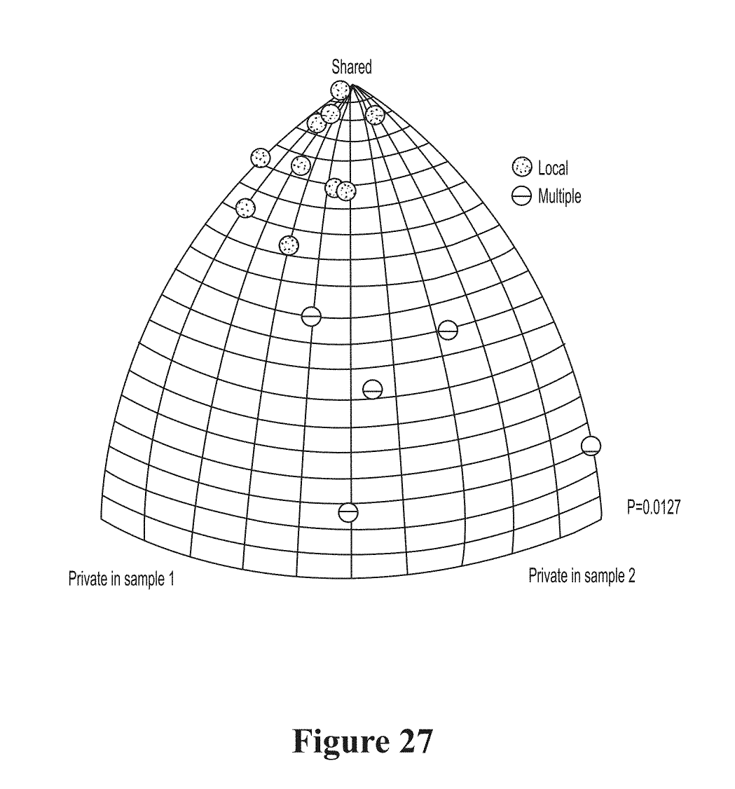

To understand the association between spatiotemporal architecture and genetic relevance, Nei's genetic distances among multisector samples from the same individual was calculated. Genetic diversity was greater in multiple tumors than in locally adjacent tissues (q=4.7×10-5, Wilcoxon rank-sum test), in distant recurrences than in local recurrences (q=1.4×10⁻⁵, Wilcoxon rank-sum test), and in long-term recurrence than in short-term recurrences (q=2.9×10⁻³, Wilcoxon rank-sum test). FIGS. 25A-25D provide comparison of genetic heterogeneity across glioma multisector and longitudinal samples. Patient samples were classified into five groups for comparative analyses: local, multiple lesion, S.T. (short-term) longitudinal local, L.T. (long-term) longitudinal local, and longitudinal distant. FIG. 25A is a graph showing Nei's genetic distances for each of the indicated groups. q values were calculated by Wilcoxon rank-sum test and corrected for false discovery rate (FDR) using the Benjamini-Hochberg method. S.T. and L.T. local correspond to short-term (<18-month surgical interval) and long-term (≧18-month interval) recurrent tumors, respectively. The lines within each ‘violin’ represent the 25th, 50th, and 75th quantiles. n=25 (local), 5 (multiple lesion), 17 (S.T. local), 3 (L.T. local), and 10 (distant). Multinomial logistic regression was applied to classify multisector sample pairs on the basis of their genomic features. This analysis highlighted that tumors from distant regions or long-term recurrences, separated by surgical intervals exceeding 18 months, constitute a distinct evolutionary scenario in GBM. FIG. 25B is a graph illustrating of leave-one-out results from multinomial logistic regression. Each point represents one pair of samples, and the coordinates correspond to the probability that the pair is local, multiple lesion/longitudinal distant, or longitudinal local. L.T. recurrent samples were classified together with multiple lesion/longitudinal distant samples, indicating that they might follow the same evolutionary model. In colorectal tumors, a Big Bang model shows that cells from different biopsies of the same tumor share clonal and subclonal variants (FIG. 25C, left). FIG. 25C is a graph showing tumor evolution behind the Big Bang and multiverse models. The Big Bang model is represented as a mixture of tumor cells that share many clonal and subclonal alterations. The multiverse model is represented by a greater proportion of private events at a clonal level. In accordance with this model, samples taken from locally adjacent tumors shared a large proportion of clonal and subclonal events. FIGS. 26A-26B show shared clonal and subclonal mutation ratios. FIG. 26A is a graph providing the percentage of clonal events shared among tumor fragments of different groups represented as violin plots. Each individual point represents a biopsy pair and lines from bottom up represent the 25^{th}, 50^{th} and 75^{th} percentiles, respectively. P values were obtained using Wilcoxon rank sum test and corrected for multiple tests using FDR (Benjamini-Hochberg). FIG. 26B is a graph providing shared sub-clonal mutation ratio among the groups. Statistics were calculated the same as in FIG. 26A. In contrast, multiple tumors contained fewer shared (more private) clonal mutations when compared to local tumors (q=1.86×10⁻³, Wilcoxon rank-sum test; FIG. 26A). This finding was corroborated by computing statistics on the space of evolutionary trees (evolutionary moduli spaces; see Methods of “The multiverse model of tumor evolution” and “Moduli space analysis”). FIG. 27 is a graph providing moduli space to represent sample pairs from the same patient. Given a pair of samples, the number of common clonal mutations (upper corner), the number of private clonal mutations in sample 1 (left corner), and the number of private clonal mutations in sample 2 (right corner) were calculated. The Ranksum test was used to measure the difference between local and multiple GBM in terms of number of common clonal mutations. Locally adjacent tumors clustered near the tip of the space, indicating a higher shared mutation ratio than that of multiple tumors (P=1.27×10-2, Wilcoxon rank-sum test). These results indicate that, in contrast to locally adjacent tumors, geographically separated multifocal tumors and/or long-term recurrent tumors are seeded from distinct clones, a phenomenon that is called the ‘multiverse model’ (FIG. 25C, right). Unlike the Big Bang model, in the multiverse model, tumor samples that are derived from different tumor masses share very few genomic alterations, indicating that tumor clones are geographically segregated at an early stage of evolution and that each clone acquires distinct private alterations, leading to the construction of multiple universes.

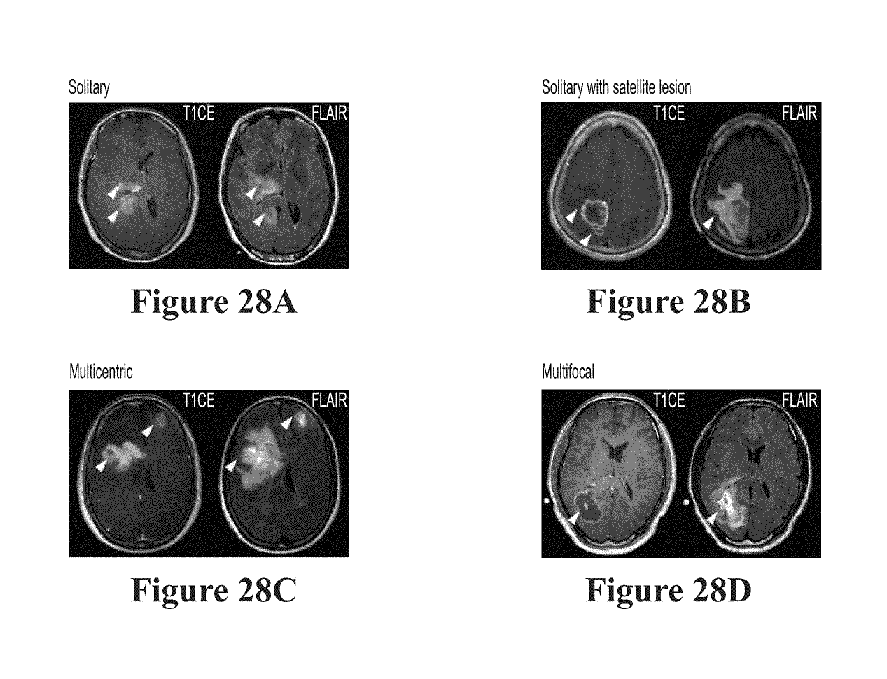

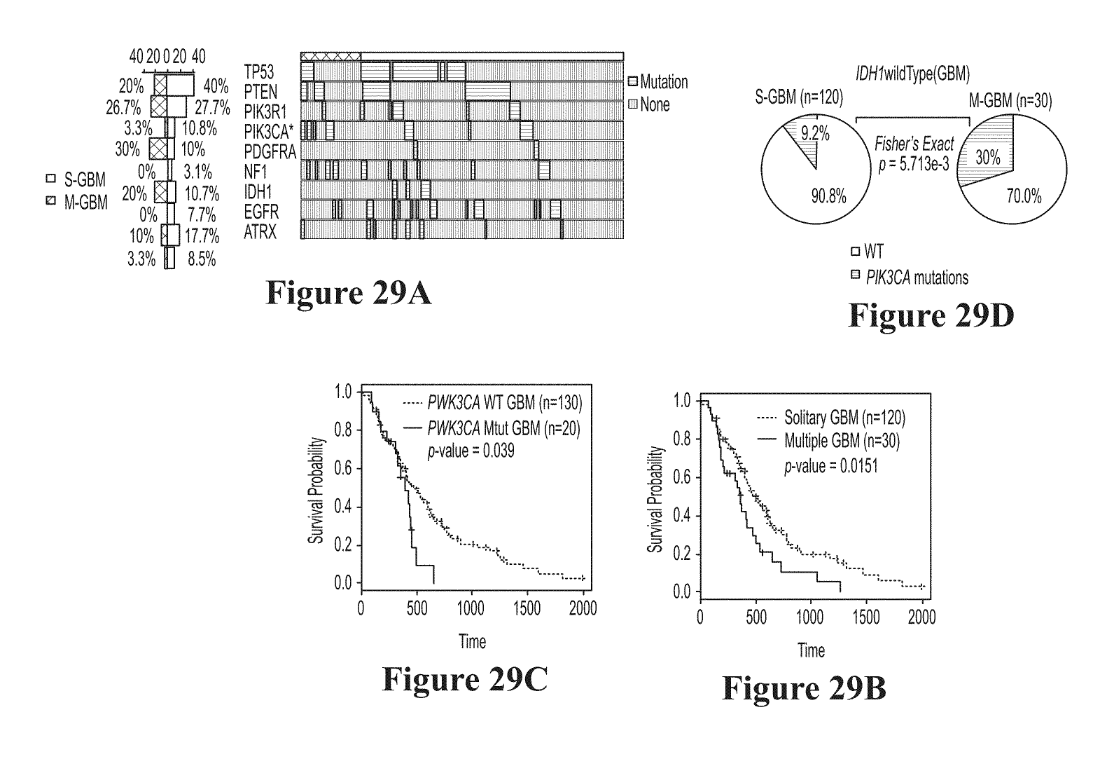

Next, the mutation profiles of GBMs with multifocal/multicentric lesions (M-GBMs) or solitary lesions (S-GBMs) in a total of 160 treatment-naïve individuals from both the SMC and TCGA (17) cohorts were investigated. FIGS. 28A-28D provide representative MRIs of GBM subgroups. FIG. 28A is an image providing typical T1CE and FLAIR MR images for solitary tumors. FIG. 28B is an image showing solitary with satellite lesions. FIG. 28C is an image showing multicentric tumors. FIG. 28D is an image showing multifocal tumors. Solitary combined with satellite lesion are grouped as “S-GBM”. Multifocal and multicentric tumors are considered as “M-GBM” following the criteria that M-GBMs were determined when the non-continues enhancements were independently presented on both T1CE and FLAIR images. FIGS. 29A-29D provide mutation profiles in multifocal/multicentric (M-) and solitary (S-) GBMs. FIG. 29A is a graph showing mutational profiles (right panel) and proportions (left panel) of representative GBM-associated genes in M- (checkered bar, n=30) or S-GMBs (white bar, n=130). Only protein changing mutations were counted (VAF>5% and read depth >X20). *p<0.05 on Fisher's Exact test. Notably, nonsynonymous mutations of PIK3CA were enriched in M-GBM (13/130 S-GBM and 9/30 M-GBM tumors; P=7.905×10⁻³, Fisher's exact test). FIG. 25D are pie charts depicting the frequencies of PIK3CA mutations in multifocal/multicentric glioblastomas (M-GBMs) (30%, 9/30) and solitary glioblastomas (S-GBMs) (10%, 13/130). WT refers to wild type. The P value was calculated using Fisher's exact test. This conclusion remained the same in the IDH1-wild-type cohort. FIG. 29B is a graph showing survival analysis of M-GBM. PIK3CA induces multipotency of mammary tumors, suggesting that it has an associative role in tumor multiplicity. Survival analysis indicated that both patients with M-GBM and PIK3CA-mutant patients had a worse prognosis than patients with S-GBM or wild-type PIK3CA (P=0.0151 and 0.039, respectively, log-rank test). FIG. 29C is a graph showing survival analysis of PIK3CA mutant GBM. p-value was calculated by ranksum test. FIG. 29D are pie charts showing enrichment of PIK3CA mutations in M-GBM in IDH1 wild type GBM.

To further characterize the heterogeneity of expression profiles, single-cell RNA-seq data from a total of seven different samples from three patients were curated. Overall, expression-based cell subtypes were not clearly determined by location or time. To make sure that this observation was not due to the limitations of this classification and to capture the transcriptional similarity among different cells, topological data analysis was used, a recently developed technique that summarizes and reduces the dimensionality of large data sets while retaining local high-dimensional structure.

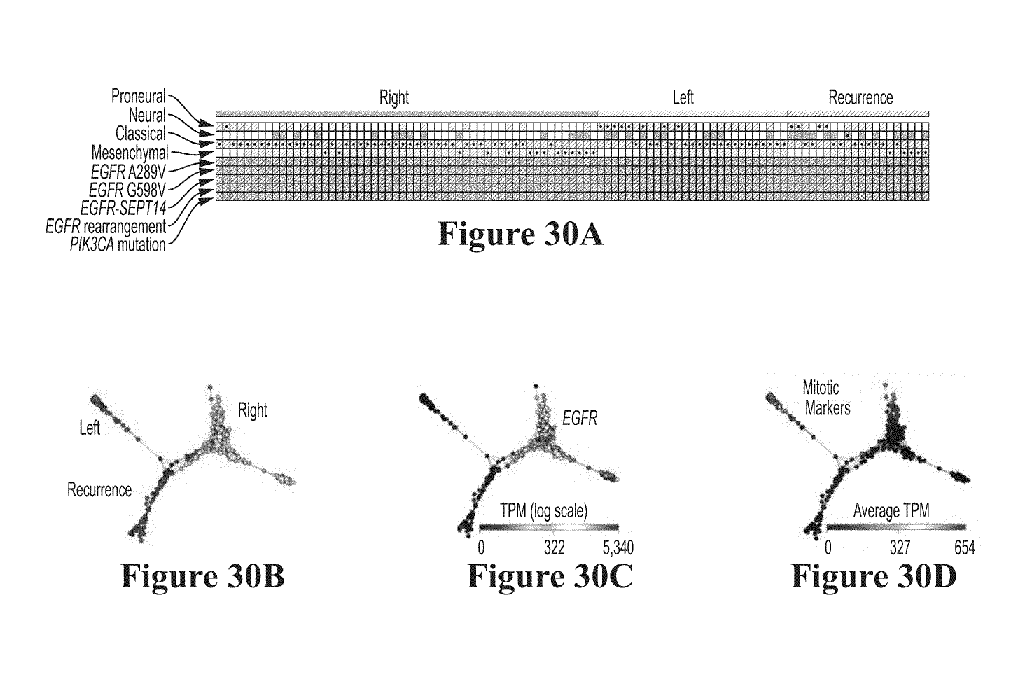

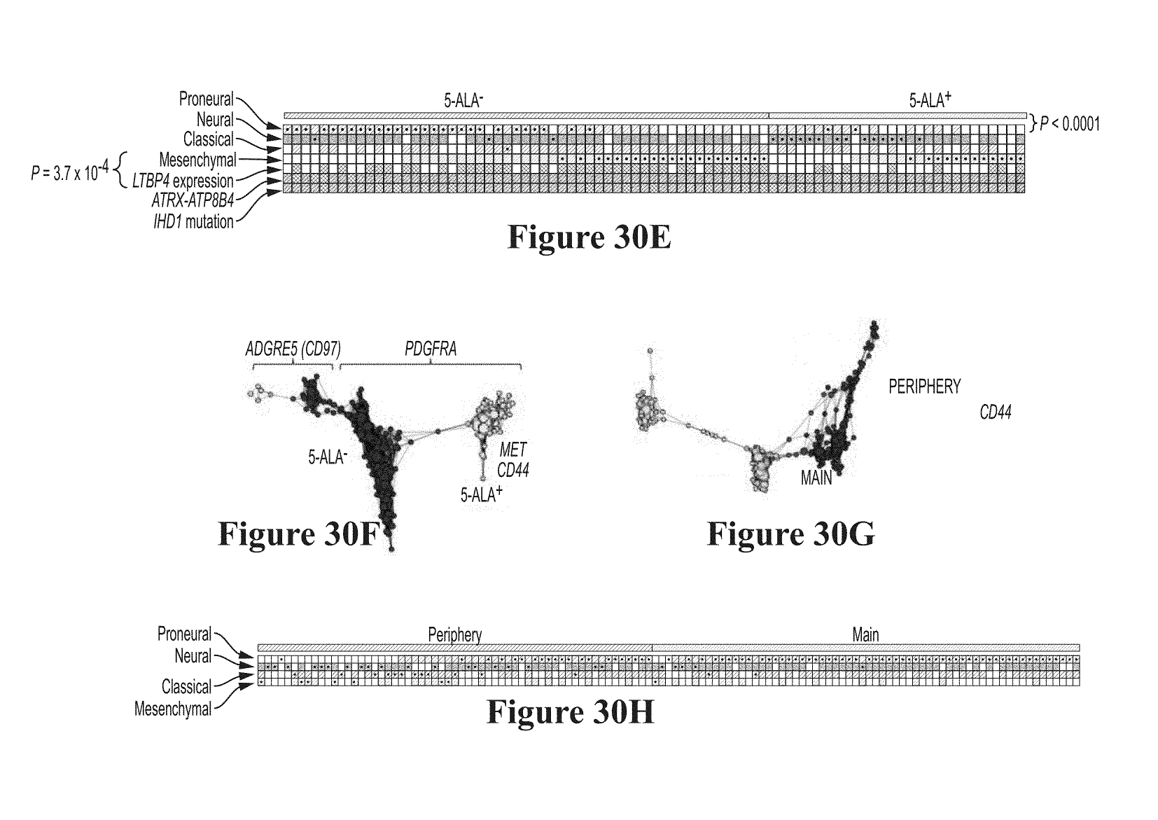

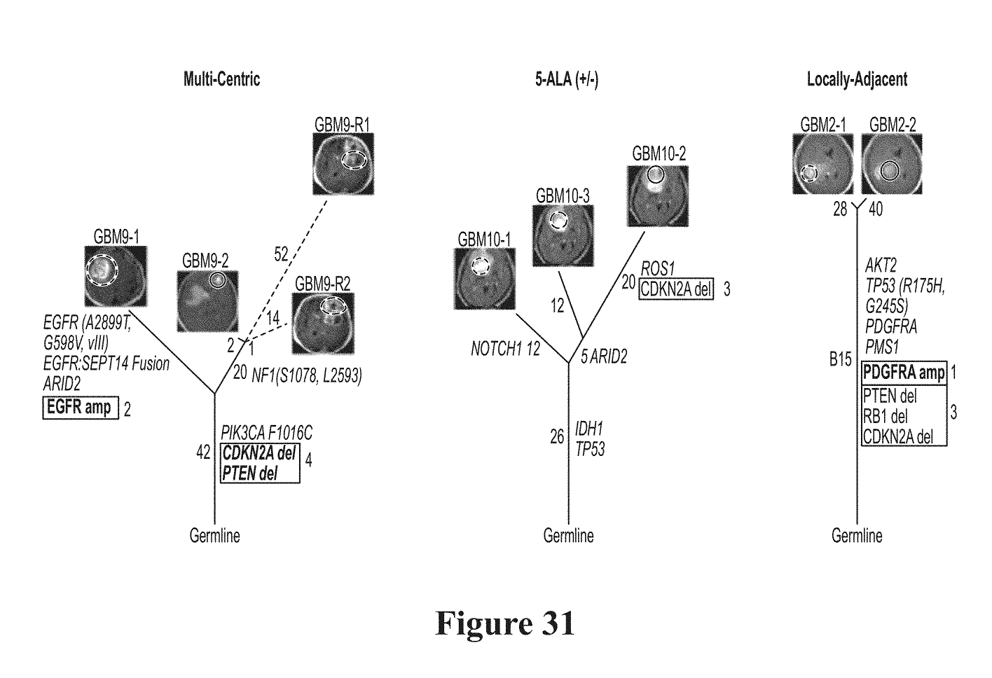

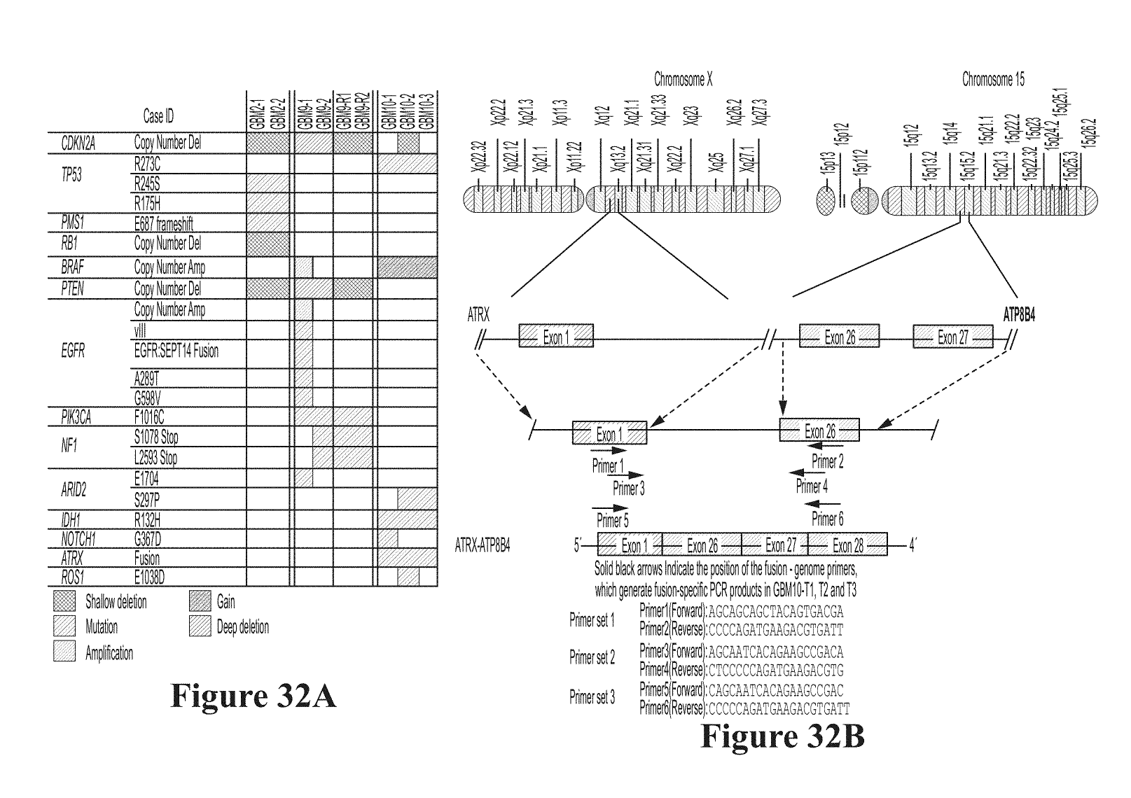

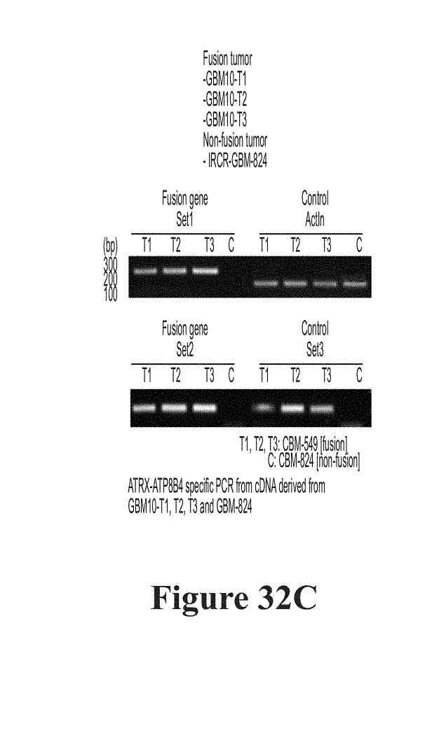

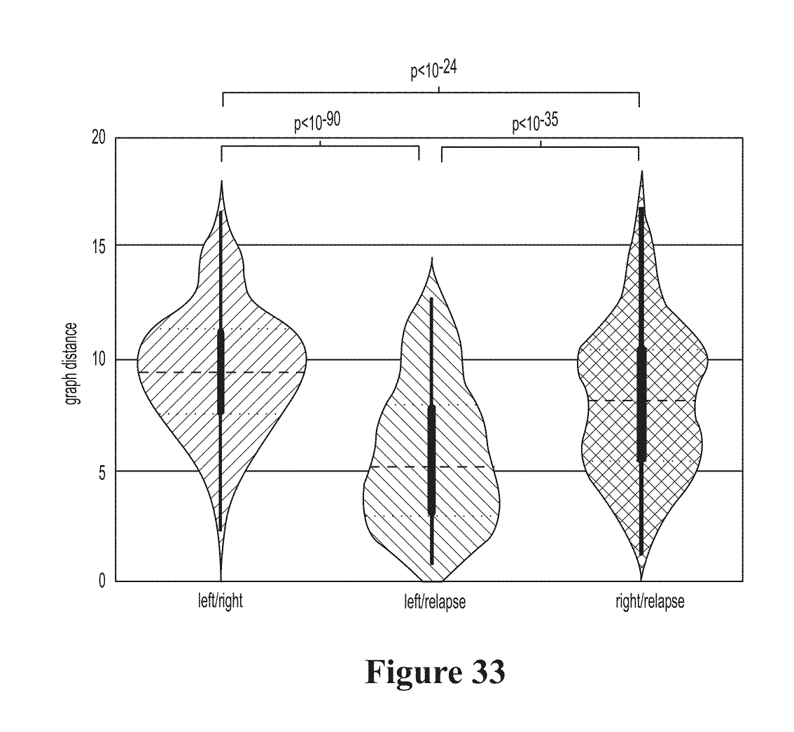

FIG. 30A is a graph providing expression profiles of individual tumor cells from three samples of GBM9 (left initial, right initial, and relapse) according to expression subtype. For each cell, the subtype with the highest expression is marked with an asterisk. Several EGFR genomic alterations could be identified in the single-cell expression data (checkered), despite the abundance of missing data (grey filled). FIG. 30B is a graph providing topological representation of the expression data of individual tumor cells from GBM9, labeled by sample of origin. Each node represents a set of cells with similar transcriptional profiles. A cell can appear in several nodes, and two nodes are connected by an edge if they have at least one cell in common. FIG. 30C is a graph providing topological representations of GBM9 where tumor cells are labeled by expression of EGFR. TPM, transcripts per million. FIG. 30D is a graph providing topological representations of GBM9 where tumor cells are labeled by expression of mitotic genes. TPM, transcripts per million. GBM9 consisted of samples from two initial tumors in the right and left frontal lobes and a recurrent tumor in the left frontal lobe that emerged after concurrent chemoradiotherapy (CCRT) and EGFR-targeted treatment. It was found in bulk whole-exome sequencing (WES) and confirmed using ultra-deep sequencing and single-cell analysis that cells from the recurrent tumor shared genomic and expression features with initial tumor cells from the left frontal lobe (FIG. 30B and FIGS. 31-33). FIG. 31 is a graph showing tumor phylogenies for GBM9 (multicentric), GBM10 (5-ALA), and GBM2 (local) with representative preoperative MR images. Circles indicate the tumor tissues that were resected. The phylogenetic tree represents diverse evolutionary patterns. For each tree, the number of somatic mutations (SNV and Indel, VAF >5% and read depth >X20) and corresponding key drivers are annotated in light purple. The length of the branch is directly proportional to the number of mutations. Genes in box 1 and box 2 represent gain and amplification, respectively (≧0.6 for gain and ≧1.5 for amplification). Genes in box 3 and box 4 represent heterozygous and homozygous deletion, respectively (≦−0.5 for heterozygous and ≦−1 homozygous deletions). FIGS. 32A-32C provide Status of somatic driver mutations, copy number alterations, and structure rearrangement (exon deletion and fusion) for multisector samples from GBM2, 9, and 10. FIG. 32A is a chart showing somatic mutations (SNV and Indel, VAF >5 and read depth >20) and (≧0.6 for gain, ≧1.5 for amplification, ≦−0.5 for shallow deletion and <−1 deep deletions). FIGS. 32B and 32C are graphs showing experimental validation of ATRX fusion in GBM10. FIG. 33 is a graph depicting distribution of average graph distances between cells in the topological representation of FIG. 30B. The graph distance between two cells on the topological representation is defined as the number of edges separating the two cells. The distance between cells from the left initial and relapse tumors is significantly lower than between cells from the right initial and relapse tumors, and between cells from the right initial and left initial tumors, showing that these tumors have more similar expression patterns. Particularly, there were 61 somatic mutations shared by the left initial tumor and the recurrent tumor, while there were only 42 shared by the right initial tumor and the recurrent tumor. Single-cell transcriptome analysis showed EGFR expression predominantly in the right tumor mass, but not in the left initial and recurrent tumors (FIG. 30C). Different single cells harbored different EGFR alterations, implying that these alterations were late events during tumor evolution. PIK3CA mutations were detected from single cells in all three samples, which is consistent with the bulk WES result that PIK3CA mutations are founder events (FIGS. 24A and 31A). Our analysis also showed the presence of transcriptional heterogeneity within the individual samples. A subset of the left initial tumor cells was characterized by upregulation of mitotic genes that was not found in either the right or recurrent sections (FIG. 30D).

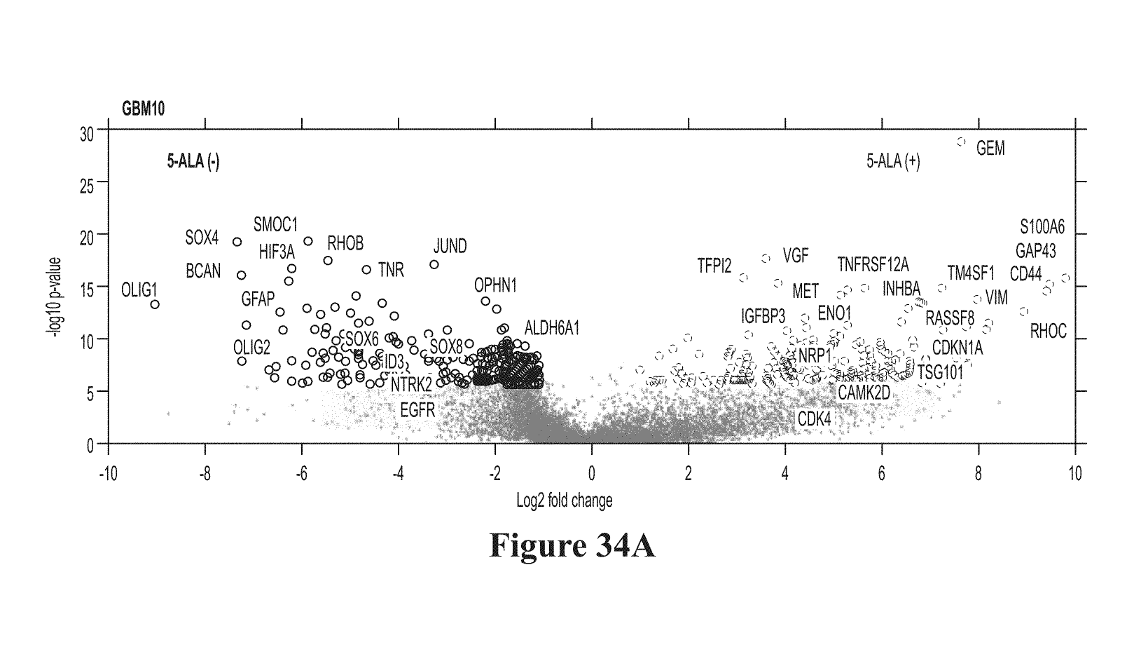

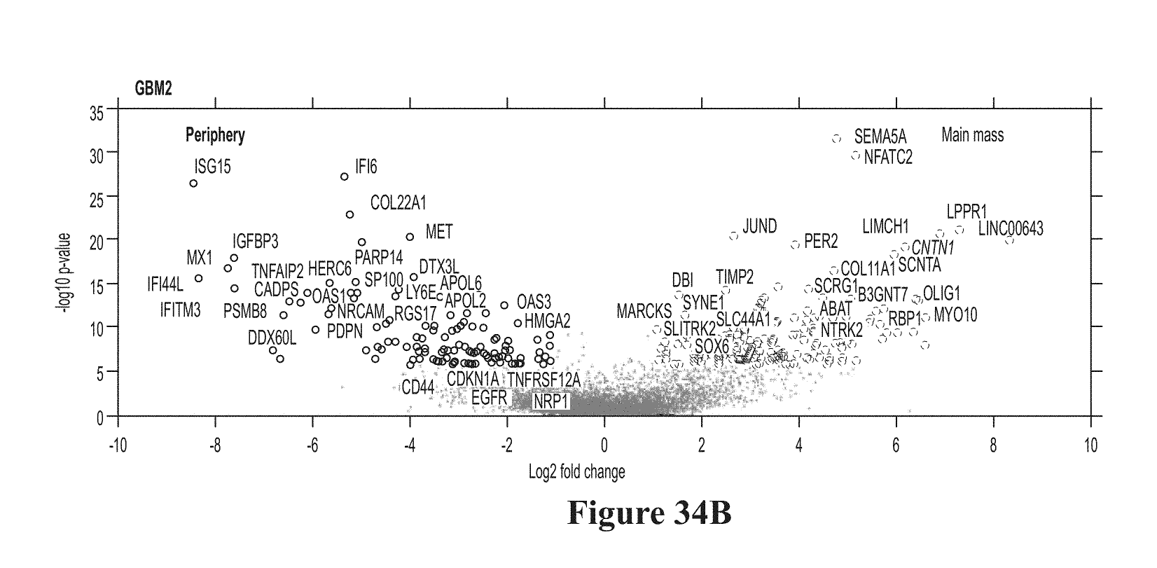

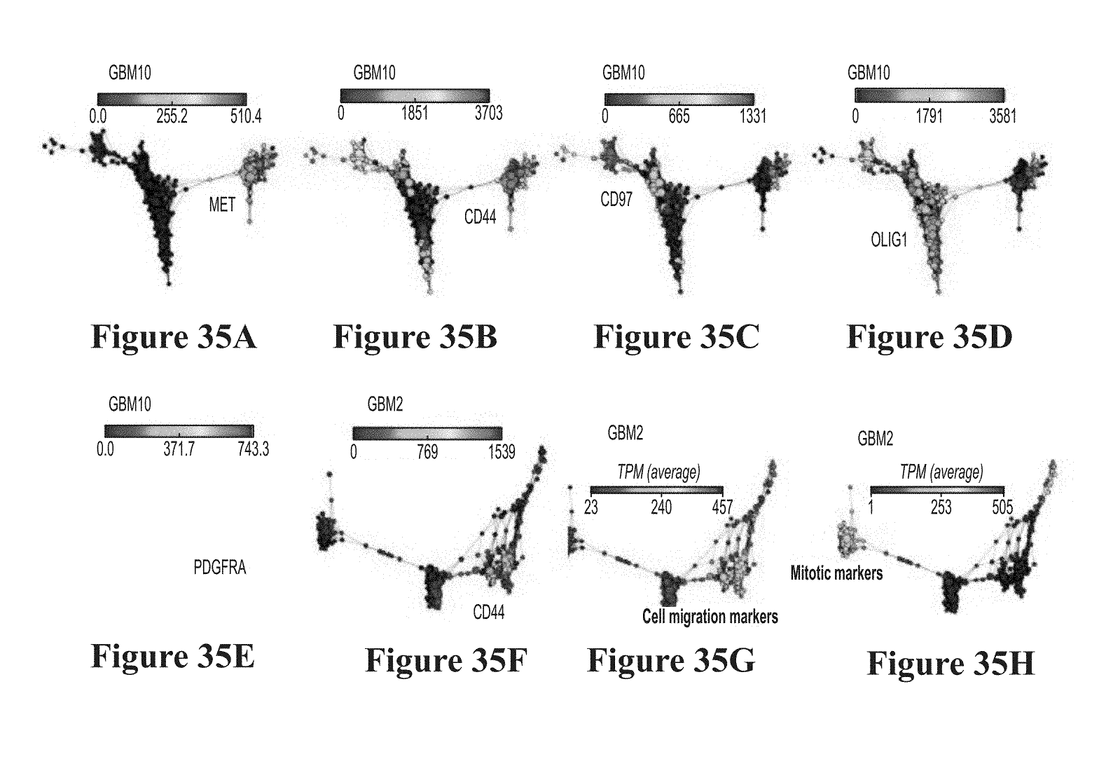

Additionally, IDH1-mutant tumor cells were profiled, which are distinguished by their 5-aminolevulinic acid (5-ALA) uptake pattern (populations stained for tumor cellularity) (GBM10; FIGS. 30E-30F, 31, and 32). FIG. 30E is a graph providing expression profiles of individual tumor cells from GBM10 (two samples: 5-ALA⁺ and 5-ALA⁻). The P value for comparison of the proneural and 5-ALA profiles was obtained using Fisher's exact test. The P value for comparison of the mesenchymal profile and LTBP4 expression was calculated using Spearman's correlation. ATRX fusion was validated by RT-PCR assays (FIGS. 32B and 32C). FIG. 30F is a graph providing topological representations of expression data for individual tumor cells from patients GBM10. Previous glioma studies suggested that a low pathologic grade is associated with a low rate of 5-ALA uptake; however, genomic determinants for 5-ALA uptake remain elusive. Using single-cell transcriptome analysis, predominant enrichment of proneural cells in the 5-ALA− sample was found, supporting previous observations that GBM cells can evolve from proneural precursors (P<0.01, Fisher's exact test; FIG. 30E). Also, enrichment of expression for several cell proliferation and migration markers in the 5-ALA+ section were found, including MET and CD44. Notably, 5-ALA− tumors, which are considered to be less aggressive, are actually fully fledged tumors that harbor driver mutations and express markers for aggressive tumors (FIGS. 30E-30F, 31B, 32, 34A, and 35). FIGS. 34A-34B show volcano plots of single cell expression. FIG. 34A is a graph showing in GBM10, single cells from sample with 5-ALA (−) that were compared with those from 5-ALA (+). FIG. 34B is a graph showing in GBM2, single cells from margin sample that were compared with those from main tumor. p-values were calculated based on t-test. FIGS. 35A-35H show topological representation of the single cell expression data from patients GBM10 and GBM2. FIG. 35A-35E show the topological representation of the expression data of tumor cells from patient GBM10 labelled by the expression level. FIG. 35A is a graph showing MET. FIG. 35B is a graph showing CD44. FIG. 35C is a graph showing CD97. FIG. 35D is a graph showing OLIG1. FIG. 35E is a graph showing PDGFRA. FIGS. 35F-35H show the topological representation of the expression data of tumor cells from patient GBM2 labelled by the expression level. FIG. 35F is a graph showing CD44. FIG. 35G is a graph showing cell migration genes. FIG. 35H is a graph showing mitotic genes. Finally, main tumor and resection margin samples from GBM2—a locally adjacent hypermutated case—were studied and distinct subpopulations of cells expressing mitotic cell markers and migration-associated genes were found, including CD44 (FIGS. 30G, 31, 32, 34, and 35). FIG. 30G is a graph showing topological representations of expression data for individual tumor cells from patients GBM2. FIG. 30H is a graph showing expression profiles of individual tumor cells from patient GBM2 shown according to GBM expression subtype. Asterisks indicate the representative subtype identities of the corresponding cells.

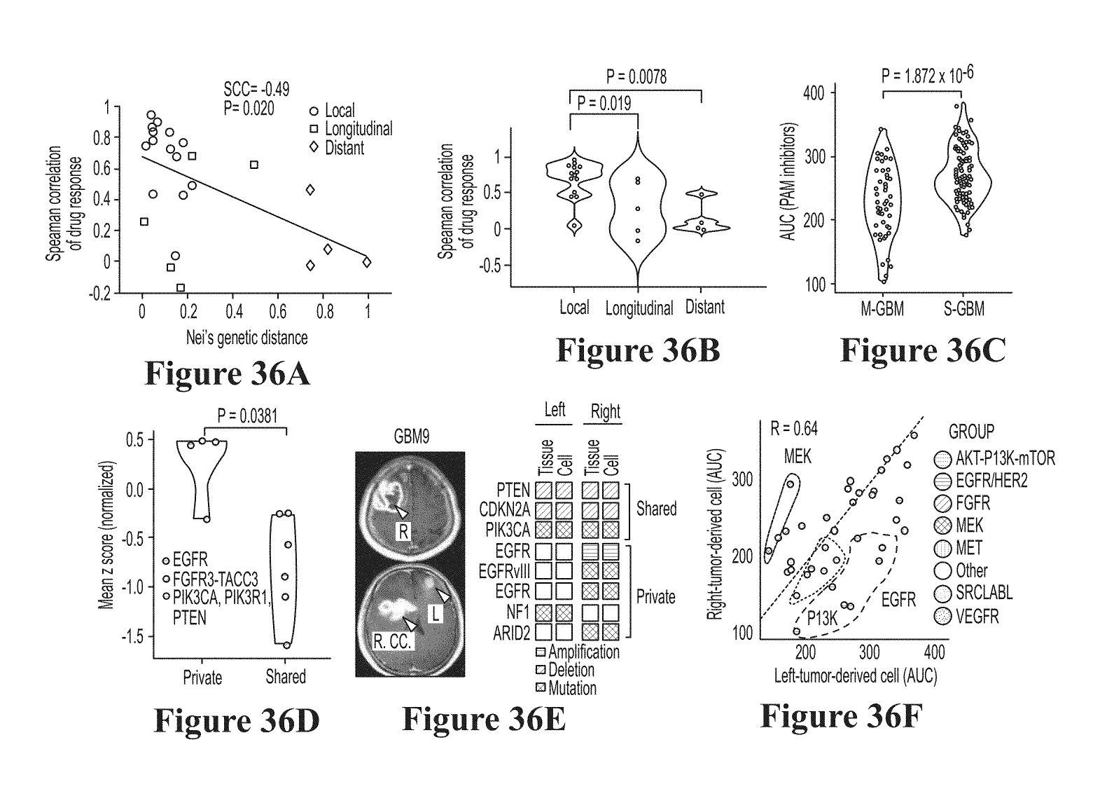

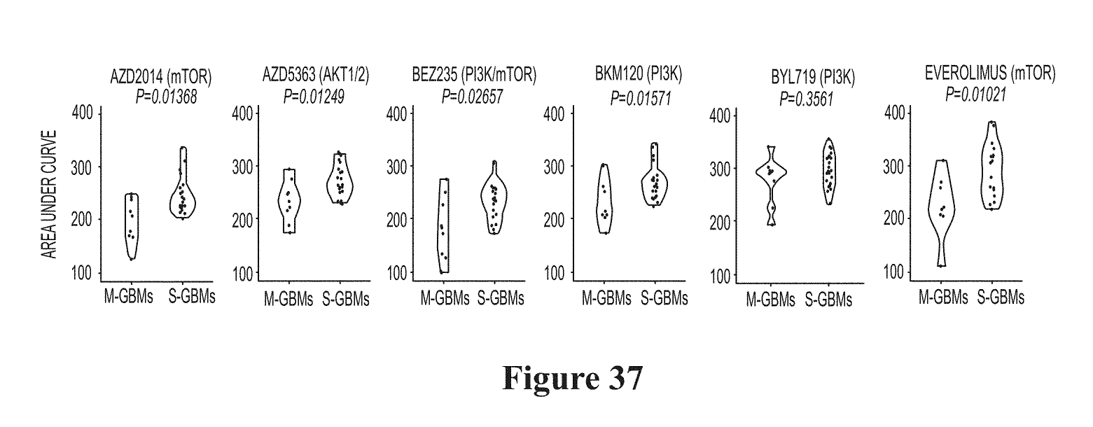

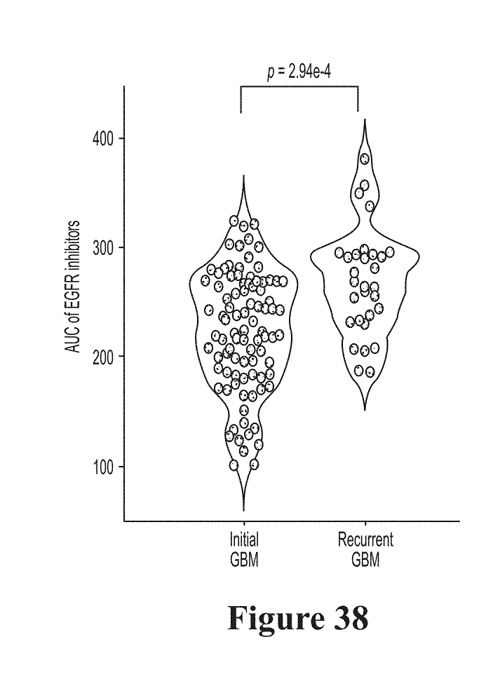

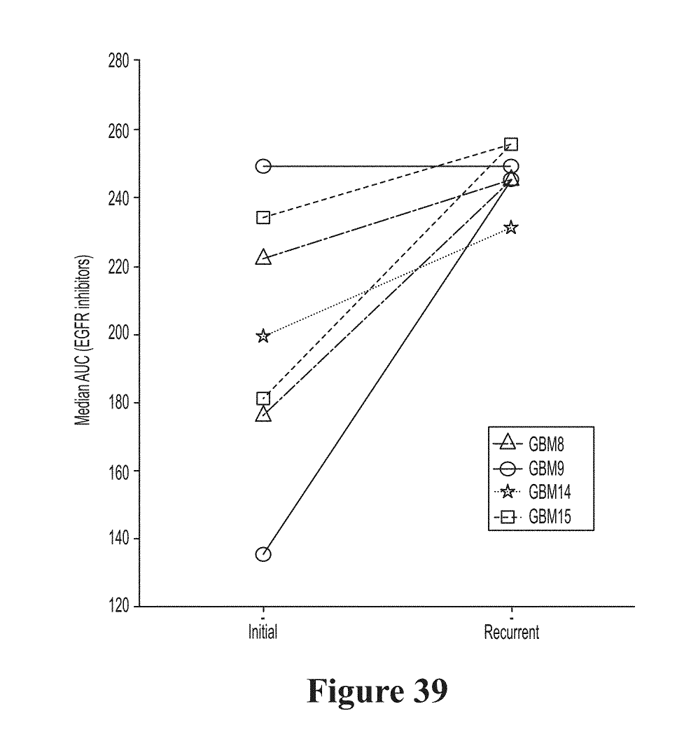

To investigate the influence of genetic heterogeneity on drug response, 28 PDCs from 11 individuals were isolated and 40 different cancer-related compounds were screened (Table 2). It was found that Nei's genetic distance was associated with drug-response correlation (P=0.02, Wilcoxon rank-sum test). FIG. 36A is a graph depicting chemical screening of multiregion PDCs. PDCs were treated with 40 chemical agents targeting oncogenic signaling pathways in dilution series from 20 μM to 4.88 nM. The x-axis shows Nei's genetic distances between fragments from the same patient, and the y-axis shows the Spearman's correlation coefficient (SCC) of corresponding fragments based on drug sensitivities as measured by area under the curve (AUC). Consistently, both distant and longitudinal samples showed significantly broader drug responses than local samples. FIG. 36B is a graph showing violin plots for the SCC of drug responses for the groups described in FIG. 36A. Each black dot represents a tumor sample. n=14 (local), 5 (longitudinal), and 4 (distant). It was found that the PDCs from M-GBMs were more sensitive to PI3K-AKT-mTOR (PAM) pathway inhibitors than PDCs from solitary tumors were (P=1.872×10-6, Wilcoxon rank-sum test; FIGS. 36C and 37). FIG. 36C is a graph showing mean values of the AUCs for six PI3K-AKT-mTOR (PAM) inhibitors (BEZ235, BKM120, BYL719, AZD5363, AZD2014, and everolimus) for PDCs isolated from M-GBMs (n=9) or S-GBMs (n=22). Each black dot represents a mean. FIG. 37 is a graph showing violin plots of AUCs against PAM drugs. AUCs for 6 PI3K-AKT-mTOR (PAM) inhibitors (AZD2014, AZD5363, BEZ235, BKM120, BYL719 and Everolimus) of PDCs from treatment naïve M- (n=9) or S- (n=22) GBM tumors. All p values were obtained by resampling-based Wilcoxon rank-sum test. This indicates that PAM inhibitors could provide a clinical benefit for patients with M-GBM. In addition, it was observed that PDCs from recurrent GBMs were more resistant to EGFR inhibitors than the initial tumor cells were (P=2.9×10-4, Wilcoxon rank-sum test; FIGS. 38 and 39). FIG. 38 is a graph showing AUC values for 6 representative EGFR inhibitors (afatinib, erlotinib, dacomitinib, gefitinib, lapatinib and neratinib) from initial or recurrent GBMs. P value was calculated using Wilcoxson Ranksum test. FIG. 39 is a graph providing median values of AUCs of 6 EGFR inhibitors against PDCs from initial and recurrent tumors of indicated patients.

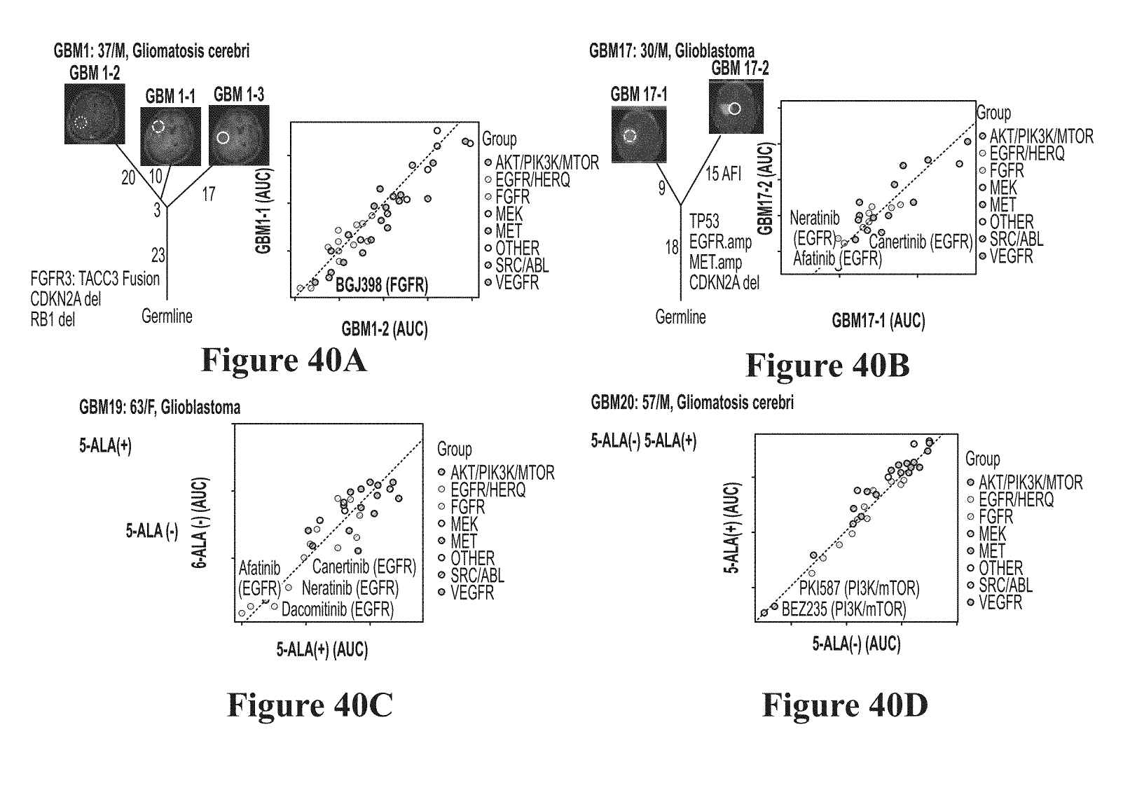

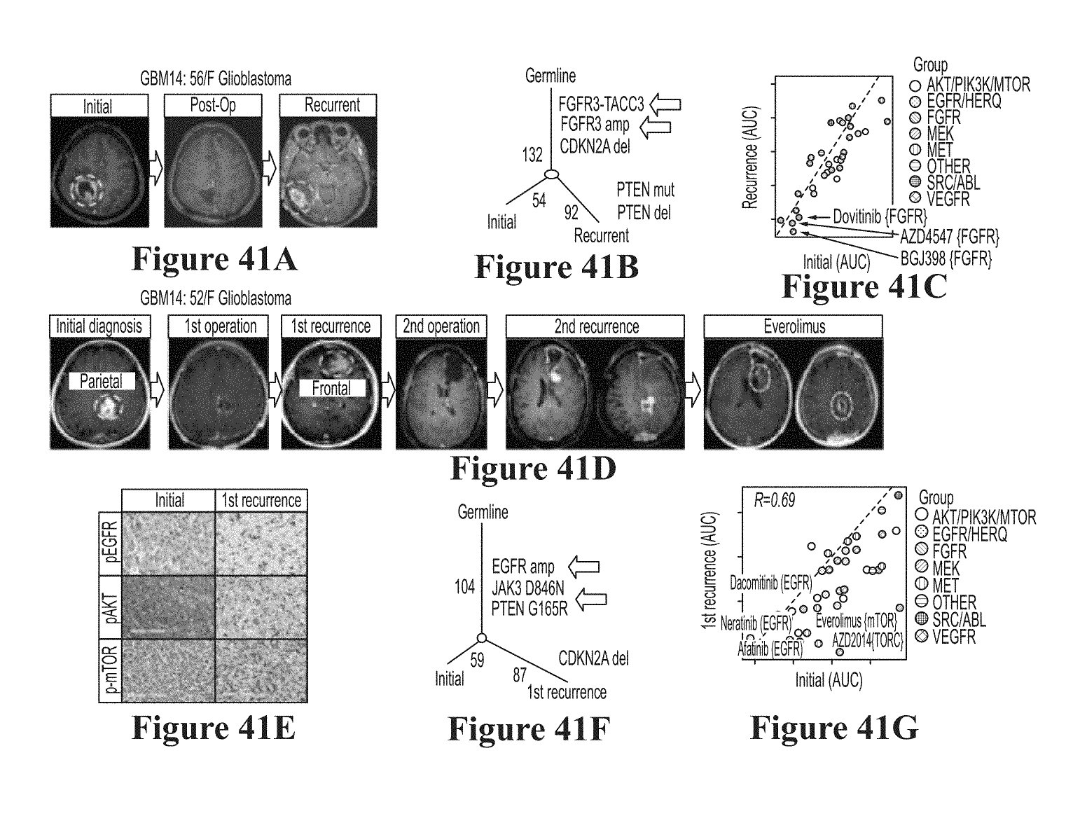

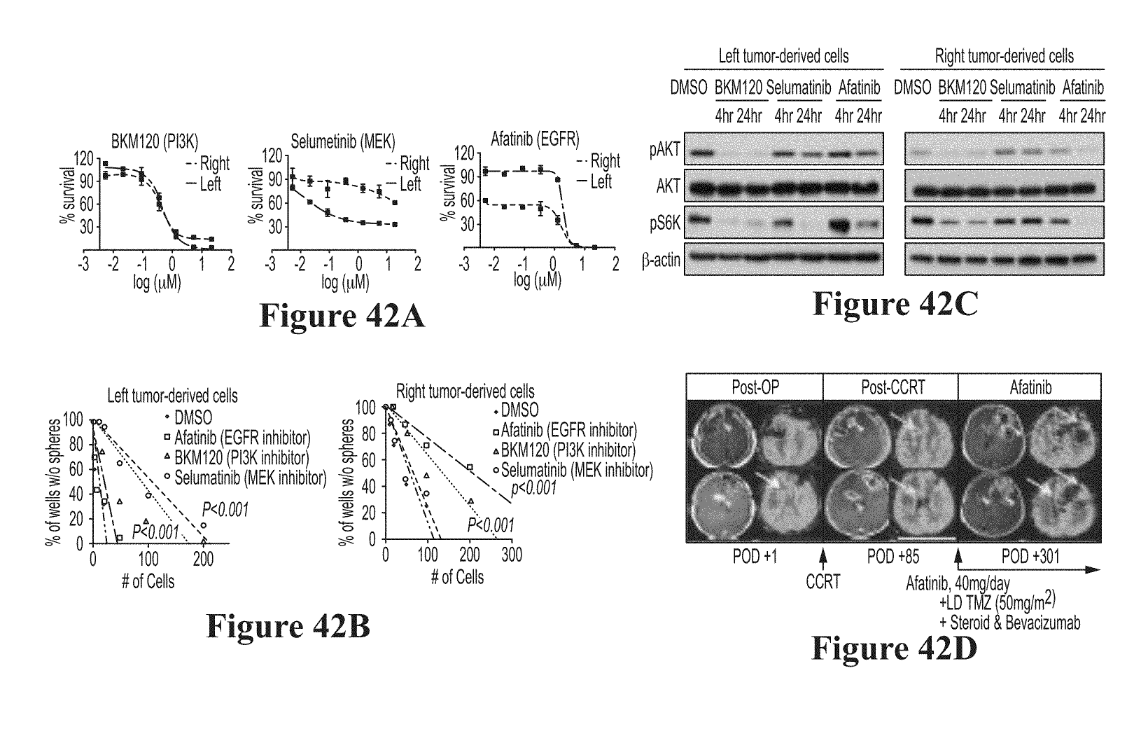

It was hypothesized that clonal alterations found in all multisector samples (truncal alterations) represent better molecular targets than those found in only a subset of multisector samples (private alterations). In agreement with this truncal-target hypothesis, multisector PDCs were more sensitive to drugs that target shared alterations than to drugs that target private alterations (P=0.0381, Wilcoxon rank-sum test; see Methods of “PDC-based chemical screening and analysis”, FIG. 36D, 40-42). FIG. 36D is a graph providing the normalized z score for each PDC obtained from the AUCs for the indicated drug classes used in FIG. 36A and FIG. 36B, which was plotted when the corresponding tissues harbored associated genetic alterations, designated as ‘shared’ or ‘private’. A genetic alteration was determined to be private when the drug-response-associated genetic alteration was private, and vice versa for the shared group. For example, PTEN mutations for P13K-AKT-mTOR pathway inhibitors were private, meaning they were exclusive to only one or two regions of the multi sector sample. FIGS. 40A-40D show drug response correlation plots using AUCs for 40 different chemicals of multisector PDCs from indicated cases. Drugs that were commonly sensitive across both multisector PDCs were marked. FIG. 40A is a graph showing GBM1. FIG. 40B is a graph showing GBM 17. FIG. 40C is a graph showing GBM19. FIG. 40D is a graph showing GBM20. FIGS. 40A and 40B further include graphs showing phylogenetic trees derived from somatic alterations in accordance with spatial difference with representative MRIs. FIG. 41A is an image depicting representative MRIs of GBM14 at pre- and post-surgical resection, and drop metastasis at ipsilateral meninges. FIG. 41B is a graph showing phylogenetic reconstruction using somatic variants. Key driver alterations including fusion and copy number amplification/deletion were displayed. FIG. 41C is a graph showing correlation plot using AUCs for 40 molecular target agents of PDCs from initial and leptomeningeal relapsed tumors. Drugs simultaneously sensitive to PDCs from initial and recurrent specimens were marked. FIG. 41D is a graph showing representative MR images of GBM15 at pre- and post-surgical resection, and 1st recurrence at distant site. FIG. 41E is a graph showing immunohistochemical analysis to analyze the activities of EGFR, AKT and mTOR, potential downstream molecules of driver genes including EGFR and PTEN. FIG. 41F is a graph depicting the evolutionary divergence of GBM15 using longitudinal pair tissues derived from somatic variants. Key driver alterations were marked. Arrows indicate potential molecular targets ubiquitously found on longitudinal pair of GBM15. FIG. 41G is a graph showing correlation plot using AUCs for 40 drugs of PDCs derived from initial or recurrent tumors of GBM15. Drugs sensitive to both PDCs were marked. FIG. 42A is a graph showing dose response curves of BKM120 (PI3K), selumetinib (MEK) and afatinib (EGFR) demonstrated in both right or left tumor-derived cells of GBM9. FIG. 42B is a graph showing limiting dilution assay (LDA) of right and left tumor-derived cells. Cells of indicated group were plated at 1-250 cells per well on a 96-well plate, and treated with DMSO as control. The percentage of wells without spheres were calculated and plotted. The p value was obtained using Extreme Limiting Dilution Analysis (ELDA). FIG. 42C is an image showing Western blot analysis to measure the activities of AKT and S6K, key downstreams of EGFR, PI3K and MEK signaling pathways. Right or left tumor derived cells were incubated with indicated drugs for 4 or 24 hours. Beta actin was used as loading control. FIG. 42D is a graph showing representative MR (T1CE) or perfusion CT images after tumor resection. In immediate postoperative Mill, enhancing portions of residual tumor were noted in the right frontal lobe and CC. The tumor in the left frontal lobe was totally removed. In a follow-up MRI taken at one month after concurrent chemoradiation therapy (CCRT) with temozolomide, remaining enhancing portions of the tumor had increased in size and relative cerebral blood volume (rCBV), suggesting progression. Afatinib (40 mg/day, orally) treatment was started four weeks after detection of recurrent tumor. In a follow-up MRI at one month after treatment with afatinib, multifocal enhancing lesions in both the right frontal lobe and CC had subsided. In an MRI obtained after four months of afatinib treatment, the lesions in the right frontal lobe and CC had further decreased in size, whereas the size of the lesion in the left frontal lobe had markedly increased. Afatinib treatment was discontinued due to the massive progression of the left tumor, and conservative treatments including steroid and two cycles of bevacizumab were administered. Grey arrowheads mark the tumor region in the T1CE MRI scan. White arrows mark the same regions in the CT images to detect rCBV. The multiverse model implies that the extensive genetic diversity of multiple tumors presents a special challenge. Accordingly, GBM9 showed a divergent genetic profile and a highly heterogeneous drug response (FIGS. 36E-36F). FIG. 36E is a graph providing preoperative T1-weighted contrast-enhanced magnetic resonance images (MRIs) and key genomic alterations found in the corresponding tumors and their derivative cells for a patient with multicentric disease (GBM9). “R” indicates right-side tumors that encompassed the right frontal lobe and corpus callosum (CC). “L” indicates the left frontal lobe tumor. The preoperative MRIs show a multifocal infiltrative lesion in both the frontal lobes and CC. FIG. 36F is a graph showing a scatterplot of the AUCs for 40 cancer-targeting compounds on GBM9 PDCs that were derived from the left- and right-side tumors in the frontal lobes. Each dot represents the AUC of a given drug. The R value was obtained as Pearson's correlation coefficient. All P values in this figure were obtained using Wilcoxon ranksum tests. PDCs from the right-side tumor were highly sensitive to EGFR inhibitors, but not to MEK inhibitors, and vice versa for the left-side tumor. However, inhibitors of the PAM pathway were ubiquitously effective, which is consistent with the hypothesis that targeting the PAM signaling pathway could be a potent option to treat M-GBMs (FIGS. 36C and 36F). Yet not all truncal alterations can serve as drug targets. For example, gatekeeper genes, which are necessary for tumor initiation but are no longer required for tumor maintenance, are not good candidates. Although targeting subclonal mutations shows a limited effect, patients might still benefit from the elimination of a subclone that has a bystander effect on surrounding cells.

In conclusion, on the basis of comprehensive bulk and single-cell analyses, a multiverse model has been proposed to interpret the evolution of multiple GBMs. It was shown that M-GBMs are more genetically diverse than locally adjacent tumors and that genetic similarity between multiregion samples is associated with consistent drug response. Specifically, an enrichment of PIK3CA mutations in M-GBMs was found, and inhibitors of the PAM pathway are found more effective in PDCs from this cohort. These findings support the truncal-target hypothesis, which states that truncal mutations can inform more effective therapies.

### Methods

Glioma Specimens and their Derivative Cells.

After receiving informed consents, glioma specimens and clinical records were obtained from patients undergoing surgery at Samsung Medical Center (SMC) or Seoul National University Hospital (SNUH) in accordance with its institutional review board (IRB file no. 2010-04-004). Surgical samples measuring approximately 5×5×5 mm³ were snap-frozen using liquid nitrogen for genomic analysis. Whole-exome and/or RNA sequencing of 33 multisector specimens from 10 glioblastoma (GBM) patients in The Cancer Genome Atlas (TCGA) cohort and 22 previously reported GBM longitudinal pairs were curated. To investigate the genomic characteristics of solitary and multifocal/multicentric GBMs, exome sequencing data for 83 and 77 tumors with matched normal DNA from the SMC and TCGA cohorts were curated, respectively. Portions of the surgical samples were enzymatically dissociated into single cells, following the procedures reported previously with modification of immune cell depletion. Tumor cells were cultured in neurobasal medium with N2 and B27 supplements (0.5x each, half of the suggested working concentration; Invitrogen), human recombinant basic fibroblast growth factor (bFGF), and epidermal growth factor (EGF; 20 ng/ml each; R&D Systems). The patient-derived cells (PDCs) used here had shown no obvious contamination of mycoplasma.

Radiological Evaluation.

Both T1-weighted contrast enhancement (T1CE) and fluid-attenuated inversion recovery (FLAIR)/T2 axial images of 160 treatment-naive GBMs (83 and 77 tumors from the SMC and TCGA cohorts, respectively) were reviewed. Magnetic resonance images (MRIs) of tumors from the TCGA cohort have been obtained from The Cancer Imaging Archive (TCIA) website. Cases with any evidence of prior neurosurgical intervention were excluded except biopsy, lack of treatment history, or loss of T1CE or FLAIR/T2 images. To distinguish the multifocal/multicentric GBMs (M-GBMs) from solitary ones (S-GBMs), annotations from the VASARI feature set for human glioma were adapted. According to the VASARI feature set, m-GBMs are defined as having at least one region of tumor, either enhancing or nonenhancing, that is not contiguous with the main lesion and is outside of the region of signal abnormality (edema) surrounding the main mass. When a FLAIR/T2 high-signal-intensity lesion resides outside of the T1CR lesion, it is considered a separate tumor focus and is counted as a multifocal tumor in our study. In contrast, tumors that present separate contrast-enhancement lesions within the FLAIR/T2 high-signal-intensity background are considered as solitary ones.

Whole-Exome Sequencing.

An Agilent SureSelect kit was used to capture the exonic DNA fragments. An Illumina HiSeq 2000 instrument was used for sequencing and generated 2×101-bp paired-end reads.

Somatic Mutation.

The sequenced reads in the FASTQ files were aligned to the human genome assembly (hg19) using Burrows-Wheeler aligner version 0.6.2. The initial alignment BAM files were subjected to conventional preprocessing before mutation calling: sorting, removing duplicated reads, locally realigning reads around potential small indels, and recalibrating base quality scores using SAMtools, Picard version 1.73, and Genome Analysis Toolkit (GATK) version 2.5.2. MuTect (version 1.1.4) and Somatic IndelDetector (GATK version 2.2) were used to make high-confidence predictions on somatic mutations from the neoplastic and non-neoplastic tissue pairs. Variant Effect Predictor (VEP) version 73 was used to annotate the called somatic mutations. Additionally, SAVI (Statistical Variant Identification) software was run to call somatic variants and indels in order to for refine the mutation calls from the above pipeline.

Copy Number.

EXCAVATOR was used to generate estimated copy number alterations in a tumor specimen in comparison with its matching, non-neoplastic part. For each gene, copy number=2x+1 was calculated, where x is the segmentation mean from EXCAVATOR, which is defined as the log 2 (fold change) in the tumor divided by the normal sample. The gene was labeled as ‘amplified’ when the copy number was 3 and ‘deleted’ when it was ≦1.

Cancer Cell Fractions and Clonality.

ABSOLUTE was run using input of genomic variants and copy number data to infer sample purity and cancer cell fractions (CCFs) and removed those that had <20% purity. Mutations were considered as clonal if they were indicated as clonal in ABSOLUTE and had a CCF of at least 80% or if they had a CCF of 100% and were not marked as clonal or subclonal. The ABSOLUTE CCF estimates with regard to hypermutated samples appeared disproportionately subclonal in sample GBM18 initial and in TCGA-14-1402 second recurrence; it was reasoned that the large mutational load might skew estimates. In hypermutated samples, treatment-associated mutation coupled with defects in mismatch repair is deemed largely responsible for a majority of observed mutations. Therefore, mutations having CCFs greater than or equal to the maximum mismatch repair CCF were marked clonal in these two samples.

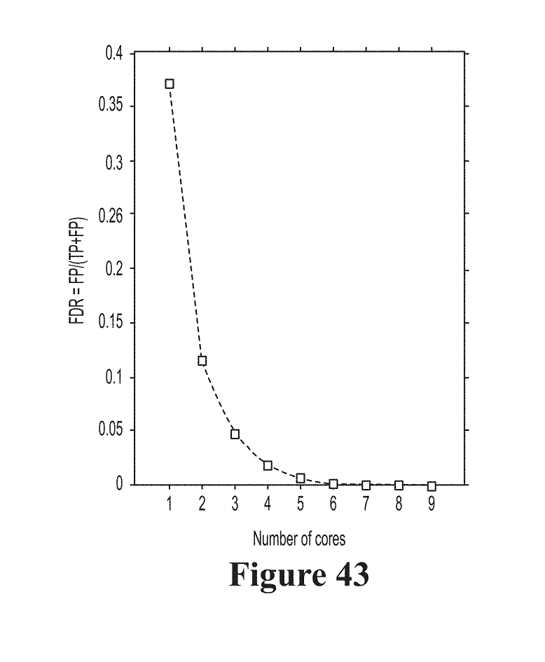

If a mutation was found to be clonal in all sectors of a patient's tumor, it was inferred to be clonal throughout the entire tumor. The number of sequenced tumor sectors or cores needed to obtain a reasonable false discovery rate (FDR) for this inference of clonality was investigated. To relate the number of sectors that were sequenced to the number of mutations deemed to be clonal tumor-wide, all possible sub sampling strategies were exhausted (number of cores k=1, 2, . . . , 9) and calculated the reported clonal mutations based on k cores. For example, if there are two cores (k=2), there are C₂⁹=36 potential sampling strategies. It was found that 22 of 36 sampling strategies contained no false discoveries in identifying clonal mutation. For each value of k, the FDR was calculated. FIG. 43 is a graph providing the correlation between the number of cores/sectors per tumor mass and false discovery rate of detecting clonal mutation. FDR=False Discovery Rate; FP=false positive; TP=true positive. Almost 90% of clonal mutations identified by two-core sequencing are true clonal mutations, and over 95% identified by three-core sequencing are true clonal mutations.

Nei's Genetic Distances.

Nei's genetic distance is used in population genetics to assess the similarity between populations, taking into account heterogeneity within populations. Samples containing the same spatial or longitudinal category (local, 5-ALA, multiple lesion, longitudinal local, longitudinal distant) were retained for statistical comparisons. Nei's genetic distance of CCF for each patient's sample was calculated as follows. Let x be all CCFs of sample 1 and y be all CCFs of sample 2:

$$D = - log \frac{\sum ( {x}_{i} {y}_{i} + ( 1 - {x}_{i} ) ( 1 - {y}_{i} ) )}{\sqrt{( \sum {x}_{i}^{2} + {( 1 - {x}_{i} )}^{2} ) ( \sum {y}_{i}^{2} + {( 1 - {y}_{i} )}^{2} )}} \qquad ( 2 )$$

The Multiverse Model of Tumor Evolution.

An increased Nei's genetic distance in multifocal/multicentric biopsies was found when compared with those that were locally adjacent. In addition, private clonal mutations appear frequently in multisectional and distant longitudinal samples, but are infrequent in local samples (FIG. 26A). This spurred a hypothesis that specific early event(s) can give rise to distinct mutational profiles in spatially separated tumors (FIG. 25A). These differences in mutational load suggested that distinct tumor profiles might arise in separate ‘universes’ of clones rather than from one large growth period followed by diversification.

For each somatic mutation, the clonal status was recorded as determined by ABSOLUTE and whether the mutation is shared or private, or the clonal status changed between biopsies. Mutations are then classified into five patterns with respect to every available pair of a patient's samples. The mutational classes were labeled as the following: CC (clonal-clonal), CS (clonal-subclonal), SS (subclonal-subclonal), CX (clonal-absent), or SX (subclonal-absent). The order of the sample pair was not important: a mutation that was clonal in one sample and subclonal in the other was marked “CS,” regardless of sample identity.

These mutational classifications were used to predict whether the spatiotemporal configuration of a sample pair fell into one of three groups: locally adjacent, local longitudinal, or multisectional/distant longitudinal. The fractions of mutations in a sample pair that fit each of the five patterns were used as features in a multinomial logistic regression. Predictions were then made using leave-one-out cross-validation.

Mutational pairs plotted on the simplex allowed visual separation multisectional/distant longitudinal, locally adjacent, or local longitudinal sections in agreement with most of our MRI classifications. The simplex axes represent the predicted probabilities of outcomes for each observation. The sample layout contained three local longitudinal outliers closest to the multisectional point of the simplex. The time interval between surgeries for the three pairs was 18 months or more. Moreover, their Nei distances were significantly different from all other sections (P=0.01652). All samples exceeding surgical intervals of 18 months were labeled as long-term recurrence and colored them in dark green. Analysis was performed in the R computing environment using the multinom function from the nnet package (see “Data availability”).

Isolation of Single Cells and RNA Sequencing.

The C1™ Single-Cell Auto Prep System (Fluidigm) was adopted with the SMARTer kit (Clontech) to generate cDNAs from single cells. Cells were captured as a single isolate on a C1 chip (17-25 μm), as determined from bright-field images obtained under 100× magnification using an Axiovert200 inverted microscope (Carl Zeiss). RNAs from pooled samples were also processed using the SMARTer kit with 10 ng of starting materials. Libraries were generated using the Nextera XT DNA Sample Prep Kit (Illumina) and sequenced on the HiSeq 2500 using the 100-bp paired-end mode of the TruSeq Rapid PE Cluster kit and TruSeq Rapid SBS kit. Before mapping RNA sequencing reads to the reference, reads were filtered out at Q33 by using Trimmomatic-0.30. Transcripts per million (TPM) values were calculated from each single cell (as if they were different samples) using RSEM (version 1.2.25) and are expressed as log 2 (1+TPM).

Gene Fusion Detection.

Chimerascan was applied to generate a list of candidate gene fusions. For bulk sequencing, only previously reported in-frame, highly expressed fusions, such as FGFR3-TACC3, MGMT fusion, EGFR-SEPT and ATRX fusion, were considered in this manuscript. For single-cell fusion analysis, if a fusion was highly expressed and independently detected in multiple cells, the fusion was reported.

Expression-Based Subtype Determination.

Gene expression was measured by RSEM and then log 2 transformed. To determine the expression-based subtype of GBM cells, z scores were first calculated for gene expression data across samples and then applied ssGSEA (version gsea2-2.2.1) to the normalized expression profile. For each cell, all genes were ranked on the basis of their expression values to create a .rnk file as input for the software GseaPreranked. An enrichment score was computed for all four subtypes initially defined in Verhaak et al. 32. The subtype with the maximal enrichment score was used as the representative subtype for each cell.

Topological Data Analysis Using Single-Cell Transcriptomes.

Normal cells were filtered out on the basis of their expression profile. To that end, the expression signatures of normal oligodendrocytes, neurons, astrocytes, microglia, endothelial cells, T cells, and other immune cells were considered, and a Gaussian mixture model was used to classify individual cells according to their expression profile. 94/133, 82/85, and 90/137 cells for GBM9, GBM10, and GBM2, respectively, were classified as tumor cells. After normalizing the gene expression level by dividing by the total number of reads in each cell to eliminate potential bias caused by batch effect, topological representations of these single-cell data were built using the Mapper algorithm, as implemented by Ayasdi. Open-source implementations of this algorithm are also available. The first two components of multidimensional scaling (MDS) were used as auxiliary functions for the algorithm. The output of Mapper is a low-dimensional network representation of the data, where nodes represent sets of cells with similar global transcriptional profiles (as measured by correlation of the expression levels of the 2,000 genes with the highest variance across each patient). Individual genes that had an expression pattern localized in the network were identified and used these to determine the subclonal structure of the samples at the level of expression.

PDC-Based Chemical Screening and Analysis.

PDCs grown in serum-free medium were seeded in 384-well plates at a density of 500 cells per well in duplicate or triplicate for each treatment. The drug panel consisted of 40 anticancer agents (Selleckchem) targeting oncogenic signals. Two hours after plating, PDCs were treated with drugs in a fourfold and seven-point serial dilution series from 20 μM to 4.88 nM using the Janus Automated Workstation (PerkinElmer). After 6 d of incubation at 37° C. in a 5% CO2 humidified incubator, cell viability was analyzed using an ATP-monitoring system based on firefly luciferase (ATPLit 1 step, PerkinElmer). The number of viable cells was estimated using the EnVision Multilabel Reader (PerkinElmer). DMSO was also included as a control in each plate. Controls were used for the calculation of relative cell viability for each plate, and normalization was performed on a per-plate basis. Dose-response curve (DRC) fitting was performed using GraphPad Prism 5 (GraphPad) and evaluated by measuring the area under the curve (AUC) of the DRC. After normalization, best-fit lines were determined and the AUC value of each curve was calculated using GraphPad Prism, ignoring regions defined by fewer than two peaks.

Cell viability was determined via calculating AUC values of DRCs with exclusion of nonconvergent fits.

Moduli Space Analysis.

To illustrate the evolution histories of tumors in GBM patients, moduli space analysis was applied in local and multiple groups of patients. Multiregion pairs were compared to calculate the number of shared and private mutations. In this analysis, clonal mutations were separated on the basis of their allele frequencies. Sector pairs were put in the left sphere on the basis of the number of shared and private mutations with high-allele-frequency mutations (>20%), whereas the same number of pairs were put in the right sphere on the basis of mutations with low-allele-frequency mutations (<20%). The same analysis was repeated using CCF instead of mutational allele fraction.

Immunohistochemistry.

Tissue specimens were fixed by formalin and embedded in paraffin. Paraffin-embedded sections were treated with 0.3% hydrogen peroxide to block endogenous peroxidase activity, and antigens were retrieved by heating sections in 10 mM sodium citrate (pH 6.0) at 95° C. for 30 min. Sections were incubated with primary antibodies overnight at 4° C., biotinylated secondary antibodies for 1 h at room temperature, and avidin—biotin complex for 1 h at room temperature.

Protein Blotting.

GBM PDCs were washed with cold PBS and harvested in lysis buffer (150 mM sodium chloride, 1% Triton X-100, 1% sodium deoxycholate, 0.1% SDS, 50 mM Tris-HCl, and 2 mM EDTA), and a protease and phosphatase inhibitor cocktail was added (Thermo Scientific). Insoluble materials were removed by centrifugation at 13,500 g for 15 min at 4° C. Proteins were separated by SDS-PAGE. Immunoblotting was performed using antibodies against indicated proteins.

Limiting Dilution Assays.

GBM PDCs were dissociated into single-cell suspensions and then plated into 96-well plates at 1-250 cells per well. Cells were incubated at 37° C. for 1-2 weeks. At the time of quantification, each well was examined for formation of neurosphere-like cell aggregates. Statistical significance was evaluated using extreme limiting dilution analysis (ELDA; Walter+Eliza Hall Bioinformatics).

Gene Fusion Validation.

Validation of gene fusion transcripts were performed by RT-PCR assays. Total RNA was extracted from tissues by AllPrep DNA/RNA Mini Kit according to the manufacturer's instructions (Qiagen). Total RNA (1 μg) was reverse transcribed to synthesize template cDNA by random hexamers using the SuperScript III First-Strand System (Life Technologies), and 20 μl of synthesized cDNA was diluted fivefold with DEPC-treated water. For RT-PCR, EzWay Taq PCR MasterMix (Komabiotech, Korea) and 5 μl of synthesized cDNA template were used. Thermal cycling was carried out under the following conditions: incubation at 95° C. for 1 min followed by 30 cycles of 30 s at 95° C., 30 s at 56° C., and 30 s at 72° C. The primer pairs used in this experiment were designed to generate the amplification product, including the breakpoints of the fusion genes. PCR products were analyzed by agarose gel electrophoresis.

Data Availability.

All sequenced data have been deposited in the European Genome-phenome Archive (EGA) with accession code EGAS00001001880 (RNA-seq and WES data).

Sample Description of Samsung Medical Center (SMC) and TCGA Cohort.

To discern whether genomic heterogeneity is linked to geographic distance, anatomical locations in 49 surgically resected tumor tissues were catalogued based on the magnetic resonance (MR) images from 20 patients (SMC cohort). Every tumor fragment along with matched blood DNA was subjected to whole exome sequencing to determine somatic variants. To investigate clonal and subclonal diversification in accordance with distinct spatial differences, fifteen distinct tumor fragments were obtained from seven different patients in locally adjacent tumor masses (GBM1-4, 7, 16, and 17) and 13 tumor fragments were collected from five different patients shown in multiple MR enhancements. Among those patients four (GBM5, 6, 8, and 18) were identified as having multifocal tumors restricted to a single hemisphere. One patient displayed a multicentric tumor (GBM9), that invaded both hemispheres. Furthermore, to evaluate genomic diversity according to 5-aminolevulinic acid (5-ALA) uptake pattern, 19 biopsies from seven patients were collected based on visible fluorescence during surgical resection (GBM10-13, 15, 19 and 20). In addition, one pair of longitudinal GBM tumors with leptomeningeal metastasis was included (GBM14) (FIG. 23A). Among the 49 tumor specimens from SMC cohort, 28 patient-derived cells (PDCs) were established for chemical sensitivity screening by 40 different cancer-targeting agents (Table 2). Topological transcriptome analysis was performed in 305 single cells isolated from 7 tissue fragments of 3 patients corresponding to locally adjacent tumor (GBM2), multifocal with recurrence (GBM9), and sections segregated by a marker of tumor cellularity (5-ALA; GBM10). Whole exome and/or RNA sequencing of 33 multisectional/longitudinal samples was curated from 10 patients inn TCGA cohort. To compare the genetic heterogeneity in longitudinal samples 22 previously reported longitudinal GBM pairs with geographic pattern at recurrence were curated.

To compare the genomic characterization between solitary (S-GBM) and multifocal/multicentric (M-GBM) tumors, whole exome sequencing of both tumor and matched normal blood in 83 and 77 treatment naïve GBMs from SMC and TCGA, respectively, were curated. In addition, 31 PDCs isolated from S- or M-GBM specimens in SMC cohort were treated with six different PI3K-AKT-mTOR (PAM) signaling pathway inhibitors, and dose response curves were constructed to derive area under curves (AUCs).

Description of Genomics, Chemical Screening and Clinical Proof-of-Principle.

GBM1 with clonal FGFR3-TACC3 fusion, was particularly sensitive to BGJ398, a selective FGFR inhibitor (FIG. 40A); GBM17 harboring EGFR amplification, in both main and periventricular specimens, showed notable sensitivity to various EGFR inhibitors (FIG. 40B); GBM19 with shared alteration of EGFR between 5-ALA (++) and (−) samples showed prominent responses on multiple EGFR inhibitors as well (FIG. 40C); GBM20, harboring shared PIK3R1 mutation between 5-ALA (+) and (−) samples, demonstrated shared and dramatic responses to PI3K/mTOR dual inhibitors (FIG. 40D).

The truncal target hypothesis was also observed in longitudinal samples; GBM14 (with common FGFR3 fusions, sensitive to three FGFR inhibitors, (FIGS. 41A-41C); GBM15 (with shared clonal EGFR and PTEN alterations showed notable sensitivity to multiple EGFR inhibitors and mTOR inhibitors (FIGS. 41D-41G)). Interestingly, GBM15 underwent another relapse on both parietal and frontal area. Everolimus, a potent mTOR inhibitor, was administered and both tumors showed remarkably response (FIG. 41D).

GBM9, a multicentric tumor, showed a successful resection of left-frontal enhancing mass however, multiple right-frontal enhancing tumors involving corpus callosum and superior frontal gyms remained (FIG. 36E). Isolated PDCs from the right side tumor were highly sensitivity to EGFR inhibitors but not to MEK pathway inhibitors and vice versa for PDCs from the left side tumor in correspondence with their genomic profiles. However, PAM pathway inhibitors were ubiquitously effective on both right and left tumor cells, consistent with previous multi-region genome profile with shared PIK3CA mutation (FIG. 36F). BKM120 (PI3K) notably decreased cell survival and clonogenic growth of both left and right tumor derived cells, whereas selumetinib (MEK) and afatinib (EGFR) were exclusively active on left and right tumor-derived cells, respectively (FIGS. 42A and 42B). The activities of AKT and S6K, crucial oncogenic signaling molecules, were notably decreased in both left and right tumor-derived cells on the treatment of BKM120 (PI3K), whereas selumetinib (MEK) and afatinib (EGFR) diminished these activities on only left or right tumor-derived cells, respectively (FIG. 42C). Clinically, the patient suffered rapid neurological deterioration and incremental increases in the sizes of the residual right-frontal tumors, accompanied by an increase in cerebral blood volume even after concurrent chemoradiation therapy (CCRT). Based on chemical screening results and successful resection of the left frontal tumor, the patient was given afatinib (40 mg/day) prior to our in-depth analyses of truncal target concept. (FIG. 42D). After 1 month of treatment, normal brain activity was restored, good cognition was evident, and residual tumors in the right frontal region and the corpus callosum exhibited partial radiological responses. MR images taken 4 months later indicated that the initial partial response of the right-frontal tumor was maintained. However, afatinib was discontinued due to progression of the left-frontal tumor, which harbored a different genomic background and responded poorly to the EGFR inhibitors upon PDC screening. This unique case entailed multicentric tumors of different genetic backgrounds, and the drug responses of the various tumors differed. The clinical failure of EGFR target therapy with massive recurrence of non-targeted tumor cells from the left frontal tumor emphasizes the need for multisector biopsies and identification of optimal ancestral molecular targets not only to eradicate the maximal tumor burden, but also to prevent the relapse of untreated clones.

TABLE 2
A list of the 40 drugs used in chemical sensitivity screens in patient
tumor-derived cells (PDCs). The chemical and/or generic names of the drugs,
their respective targets, and clinical phases were catalogued.

| No. | Compound name | Generic name | Target | Clinical Phase |
|---|---|---|---|---|
| 1 | ABT-199 | GDC-0199 | Bcl-2 | Phase 3 |
| 2 | BIBW2992 | Afatinib | EGFR | FDA Approved |
| 3 | AG013736 | Axitinib | VEGFR1/2/3, PDGFRβ | FDA Approved |
| | | | and c-Kit | |
| 4 | AZD2014 | | mTOR | Phase 2 |
| 5 | AZD4547 | | FGFR1/2/3 | Phase 2/3 |
| 6 | AZD5363 | | Akt1/2/3 | Phase 2 |
| 7 | AZD6244 | Selumetinib | MEK1 | Phase 3 |
| 8 | BEZ235 | | PI3K/mTOR | Phase 2 |
| 9 | BGJ398 | | FGFR1/2/3 | Phase 2 |
| 10 | BKM120 | Buparlisib | PI3K | Phase 2 |
| 11 | BMS-599626 | | EGFR | Phase 1 |
| 12 | bosutinib | Bosutinib | dual Src/Abl | FDA Approved |
| 13 | BYL719 | | PI3K | Phase 2 |
| 14 | XL184 | Cabozantinib | VEGFR2, c-Met, Ret, Kit, | FDA Approved |
| | | | Flt-1/3/4, Tie2 | |
| 15 | AZD2171 | Cediranib | VEGFR, Flt | Phase 3 |
| 16 | CI-1033 | Canertinib | EGFR, HER2 | Phase 3 |
| 17 | CO-1686 | | EGFR | Phase 2 |
| 18 | PF-02341066 | Crizotinib | Met, ALK | FDA Approved |
| 19 | PF299804 | Dacomitinib | EGFR | Phase 2 |
| 20 | BMS-354825 | Dasatinib | Bcr-Abl | FDA Approved |
| 21 | TKI-258 | Dovitinib | Flt3, c-Kit, FGFR1/3, | Phase 4 |
| | | | VEGFR1/2/3, PDGFR | |
| 22 | | Erlotinib | HER1/EGFR | FDA Approved |
| 23 | RAD001 | Everolimus | mTOR | FDA Approved |
| 24 | XL880 | Foretinib | HGFR and VEGFR, mostly | Phase 2 |
| | | | for Met and KDR | |
| 25 | | Gefitinib | EGFR | FDA Approved |
| 26 | | Ibrutinib | Btk, Bmx, CSK, FGR, BRK, HCK, less | FDA Approved |
| | | | potent to EGFR, ErbB2, JAK3 | |
| 27 | sti571 | Imatinib | v-Abl, c-Kit and PDGFR | FDA Approved |
| 28 | INCB28060 | Capmatinib | Met | Phase 1 |
| 29 | | Lapatinib | EGFR | FDA Approved |
| 30 | HKI-272 | Neratinib | EGFR | FDA Approved |
| 31 | AZD2281 | Olaparib | PARP1/2 | Phase 3 |
| 32 | | Pazopanib | VEGFR1/2/3, PDGFR, FGFR, c-Kit | FDA Approved |
| 33 | PF-05212384 | | PI3K/mTOR | Phase 2 |
| | (PKI-587) | | | |
| 34 | | Ruxolitinib | JAK1/2 | FDA Approved |
| 35 | | Sunitinib | VEGFR2 and PDGFRβ | FDA Approved |
| 36 | MLN518 | Tandutinib | FLT3, PDGFR, and KIT | Phase 2 |
| 37 | AV-951 | Tivozanib | VEGFR, c-Kit, PDGFR | Phase 3 |
| 38 | | Trametinib | MEK1/2 | FDA Approved |
| 39 | ZD6474 | Vandetanib | VEGFR2 | FDA Approved |
| 40 | XL147 | | PI3K | Phase 1/2 |

### REFERENCES

1. Hamburg, M. A. & Collins, F. S. The path to personalized medicine. N. Engl. J. Med. 363, 301-304 (2010).
2. Brennan, C. W. et al. The somatic genomic landscape of glioblastoma. Cell 155, 462-477 (2013).
3. Ceccarelli, M. et al. Molecular profiling reveals biologically discrete subsets and pathways of progression in diffuse glioma. Cell 164, 550-563 (2016).
4. Cloughesy, T. F., Cavenee, W. K. & Mischel, P. S. Glioblastoma: from molecular pathology to targeted treatment. Annu. Rev. Pathol. 9, 1-25 (2014).
5. Frattini, V. et al. The integrated landscape of driver genomic alterations in glioblastoma. Nat. Genet. 45, 1141-1149 (2013).
6. Snuderl, M. et al. Mosaic amplification of multiple receptor tyrosine kinase genes in glioblastoma. Cancer Cell 20, 810-817 (2011).
7. Szerlip, N. J. et al. Intratumoral heterogeneity of receptor tyrosine kinases EGFR and PDGFRA amplification in glioblastoma defines subpopulations with distinct growth factor response. Proc. Natl. Acad. Sci. USA 109, 3041-3046 (2012).
8. Kim, H. et al. Whole-genome and multisector exome sequencing of primary and post-treatment glioblastoma reveals patterns of tumor evolution. Genome Res. 25, 316-327 (2015).
9. Sottoriva, A. et al. Intratumor heterogeneity in human glioblastoma reflects cancer evolutionary dynamics. Proc. Natl. Acad. Sci. USA 110, 4009-4014 (2013).
10. Patel, A. P. et al. Single-cell RNA-seq highlights intratumoral heterogeneity in primary glioblastoma. Science 344, 1396-1401 (2014).
11. Kumar, A. et al. Deep sequencing of multiple regions of glial tumors reveals spatial heterogeneity for mutations in clinically relevant genes. Genome Biol. 15, 530 (2014).
12. Johnson, B. E. et al. Mutational analysis reveals the origin and therapy-driven evolution of recurrent glioma. Science 343, 189-193 (2014).
13. Yates, L. R. et al. Subclonal diversification of primary breast cancer revealed by multiregion sequencing. Nat. Med. 21, 751-759 (2015).
14. Wang, J. et al. Clonal evolution of glioblastoma under therapy. Nat. Genet. 48, 768-776 (2016).
15. Ohka, F., Natsume, A. & Wakabayashi, T. Current trends in targeted therapies for glioblastoma multiforme. Neurol. Res. Int. 2012, 878425 (2012).
16. Kim, J. et al. Spatiotemporal evolution of the primary glioblastoma genome. Cancer Cell 28, 318-328 (2015).
17. Liu, Q. et al. Genetic, epigenetic, and molecular landscapes of multifocal and multicentric glioblastoma. Acta Neuropathol. 130, 587-597 (2015).
18. Singh, D. et al. Transforming fusions of FGFR and TACC genes in human glioblastoma. Science 337, 1231-1235 (2012).
19. Hartmann, C., Bartels, G., Gehlhaar, C., Holtkamp, N. & von Deimling, A. PIK3CA mutations in glioblastoma multiforme. Acta Neuropathol. 109, 639-642 (2005).
20. Sottoriva, A. et al. A Big Bang model of human colorectal tumor growth. Nat. Genet. 47, 209-216 (2015).
21. Koren, S. et al. PIK3CA_{H1047R} induces multipotency and multi-lineage mammary tumours. Nature 525, 114-118 (2015).
22. Carlsson, G. Topology and data. Bull. Amer. Math. Soc. 46, 255-308 (2009).
23. Singh, G., Mémoli, F. & Carlsson, G. E. Topological methods for the analysis of high dimensional data sets and 3D object recognition. in SPBG (eds. Botsch, M. & Pajarola, R.) 91-100 (Citeseer, 2007).
24. Moiyadi, A., Syed, P. & Srivastava, S. Fluorescence-guided surgery of malignant gliomas based on 5-aminolevulinic acid: paradigm shifts but not a panacea. Nat. Rev. Cancer 14, 146 (2014).
25. Stummer, W. et al. Fluorescence-guided surgery with 5-aminolevulinic acid for resection of malignant glioma: a randomised controlled multicentre phase III trial. Lancet Oncol. 7, 392-401 (2006).
26. Ozawa, T. et al. Most human non-GCIMP glioblastoma subtypes evolve from a common proneural-like precursor glioma. Cancer Cell 26, 288-300 (2014).
27. Merzak, A., Koocheckpour, S. & Pilkington, G. J. CD44 mediates human glioma cell adhesion and invasion in vitro. Cancer Res. 54, 3988-3992 (1994).
28. Yoshida, T., Matsuda, Y., Naito, Z. & Ishiwata, T. CD44 in human glioma correlates with histopathological grade and cell migration. Pathol. Int. 62, 463-470 (2012).
29. Joo, K. M. et al. MET signaling regulates glioblastoma stem cells. Cancer Res. 72, 3828-3838 (2012).
30. Lemmon, M. A. & Schlessinger, J. Cell signaling by receptor tyrosine kinases. Cell 141, 1117-1134 (2010).
31. Kinzler, K. W. & Vogelstein, B. Lessons from hereditary colorectal cancer. Cell 87, 159-170 (1996).
32. Verhaak, R. G. et al. Integrated genomic analysis identifies clinically relevant subtypes of glioblastoma characterized by abnormalities in PDGFRA, IDH1, EGFR, and NF1. Cancer Cell 17, 98-110 (2010).
33. Lee, J. et al. Tumor stem cells derived from glioblastomas cultured in bFGF and EGF more closely mirror the phenotype and genotype of primary tumors than do serum-cultured cell lines. Cancer Cell 9, 391-403 (2006).
34. Clark, K. et al. The Cancer Imaging Archive (TCIA): maintaining and operating a public information repository. J. Digit. Imaging 26, 1045-1057 (2013).
35. Wangaryattawanich, P. et al. Multicenter imaging outcomes study of The Cancer Genome Atlas glioblastoma patient cohort: imaging predictors of overall and progression-free survival. Neuro-oncol. 17, 1525-1537 (2015).
36. Rios Velazquez, E. et al. Fully automatic GBM segmentation in the TCGA-GBM dataset: Prognosis and correlation with VASARI features. Sci. Rep. 5, 16822 (2015).
37. Giannopoulos, S. & Kyritsis, A. P. Diagnosis and management of multifocal gliomas. Oncology 79, 306-312 (2010).
38. Kyritsis, A. P., Levin, V. A., Yung, W. K. & Leeds, N. E. Imaging patterns of multifocal gliomas. Eur. J. Radiol. 16, 163-170 (1993).
39. Zinn, P. O. et al. Radiogenomic mapping of edema/cellular invasion MRI-phenotypes in glioblastoma multiforme. PLoS One 6, e25451 (2011).
40. Li, Y. M., Suki, D., Hess, K. & Sawaya, R. The influence of maximum safe resection of glioblastoma on survival in 1229 patients: can we do better than gross-total resection? J. Neurosurg. 124, 977-988 (2016).
41. Carter, S. L. et al. Absolute quantification of somatic DNA alterations in human cancer. Nat. Biotechnol. 30, 413-421 (2012).
42. Suzuki, H. et al. Mutational landscape and clonal architecture in grade II and III gliomas. Nat. Genet. 47, 458-468 (2015).
43. Li, B. & Dewey, C. N. RSEM: accurate transcript quantification from RNA-Seq data with or without a reference genome. BMC Bioinformatics 12, 323 (2011).
44. Iyer, M. K., Chinnaiyan, A. M. & Maher, C. A. ChimeraScan: a tool for identifying chimeric transcription in sequencing data. Bioinformatics 27, 2903-2904 (2011).
45. Cahoy, J. D. et al. A transcriptome database for astrocytes, neurons, and oligodendrocytes: a new resource for understanding brain development and function. J. Neurosci. 28, 264-278 (2008).
46. Butovsky, O. et al. Identification of a unique TGF-111-dependent molecular and functional signature in microglia. Nat. Neurosci. 17, 131-143 (2014).
47. Bhasin, M. et al. Bioinformatic identification and characterization of human endothelial cell-restricted genes. BMC Genomics 11, 342 (2010).
48. Chtanova, T. et al. Identification of T cell-restricted genes, and signatures for different T cell responses, using a comprehensive collection of microarray datasets. J. Immunol. 175, 7837-7847 (2005).
49. Abbas, A. R. et al. Immune response in silico (IRIS): immune-specific genes identified from a compendium of microarray expression data. Genes Immun. 6, 319-331 (2005).
50. Huang, S. & Pang, L. Comparing statistical methods for quantifying drug sensitivity based on in vitro dose-response assays. Assay Drug Dev. Technol. 10, 88-96 (2012).
51. Zairis, S. et al. Moduli spaces of phylogenetic trees describing tumor evolutionary patterns. Lect. Notes Comput. Sci. 8609, 528-539 (2014).

The contents of all figures and all references, patents and published patent applications and Accession numbers cited throughout this application are expressly incorporated herein by reference.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the systems and methods of the disclosed subject matter without departing from the spirit or scope of the disclosed subject matter. Thus, it is intended that the disclosed subject matter include modifications and variations that are within the scope of the appended claims and their equivalents.

What is claimed is:

1. A method for treating a subject with multifocal/multicentric glioblastoma (M-GBM) comprising:
obtaining at least two M-GBM tumor samples from different locations within the subject;
extracting genomic DNA from each of the at least two tumor samples to obtain at least two corresponding extracted genomic DNA samples;
determining whether the subject has a PI3K-AKT-mTOR (PAM) pathway mutation in each of the at least two extracted genomicDNA samples; and
if a PAM pathway mutation is determined in each of the at least two DNA samples, treating the subject with an effective amount of a PAM pathway inhibiting agent.

2. The method of claim 1, wherein the mutation in the PAM pathway is a gain-of-function mutation that activates the PAM pathway.

3. The method of claim 1, wherein the mutation is in PIK3CA gene.

4. The method of claim 3, wherein the mutation is at amino acid 4, 364, 1016, 1035, or 1043 of PIK3CA protein, or at equivalent positions of homologous sequences thereto.

5. The method of claim 4, wherein the mutation is selected from the group consisting of R4Q, G364R, F1016C, A1035V, M1043I, and M1043V.

6. The method of claim 1, wherein the mutation is in one or more of AKT1, AKT2, AKT3, and/or mTOR genes.

7. The method of claim 1, wherein the agent is selected from the group consisting of BKM120 (Buparlisib), XL147 (Pilaralisib), GDC0941 (Pictilisib), BYL719 (Alpelisib), GDC0032 (Tazelisib), NVP-BEZ235, LY3023414, GSK2126458, BEZ235, PF-05212384 (PKI-587), AZD5363, MK-2206, GSK21411795 (Uprosertib), GDC-0068 (Lpatasertib), LNK128, AZD2014, AZD8055, MLN0138, CC-223, RAD001 (Everolimus), rapamycin (Sirolimus), CCI-779 (Temsirolimus), AP23573 (Ridaforolimus), and combinations thereof.

8. The method of claim 1, wherein the agent is administered orally.

9. The method of claim 1, wherein the agent is administered intravenously.

10. The method of claim 1, wherein the agent comprises a nucleic acid that specifically binds to a nucleic acid encoding PIK3CA, and reduces P13K expression and/or activity.

11. The method of claim 10, wherein the agent comprises a microRNA (miRNA) molecule, small interfering RNA (siRNA) molecule, short hairpin RNA (shRNA) molecule, catalytic RNA molecule, and/or catalytic DNA molecule.

12. A method of treating M-GBM in a subject, comprising administering, to the subject, an effective amount of a PAM pathway inhibiting agent.

13. The method of claim 12, wherein the agent is selected from the group consisting of BKM120 (Buparlisib), XL147 (Pilaralisib), GDC0941 (Pictilisib), BYL719 (Alpelisib), GDC0032 (Tazelisib), NVP-BEZ235, LY3023414, GSK2126458, BEZ235, PF-05212384 (PKI-587), AZD5363, MK-2206, GSK21411795 (Uprosertib), GDC-0068 (Lpatasertib), LNK128, AZD2014, AZD8055, MLN0138, CC-223, RAD001 (Everolimus), rapamycin (Sirolimus), CCI-779 (Temsirolimus), AP23573 (Ridaforolimus), and combinations thereof.

14. The method of claim 12, wherein the agent comprises a nucleic acid that specifically binds to a nucleic acid encoding PIK3CA, and reduces PI3K expression and/or activity.

15. The method of claim 14, wherein the agent comprises a microRNA (miRNA) molecule, small interfering RNA (siRNA) molecule, short hairpin RNA (shRNA) molecule, catalytic RNA molecule, and/or catalytic DNA molecule.

16. The method of claim 12, further comprising administering to the subject an additional therapeutic agent, a stabilizing compound, and/or a biocompatible pharmaceutical carrier.

17. A kit for determining the presence of a PI3K-AKT-mTOR (PAM) pathway mutation in a subject with multifocal/multicentric glioblastoma (M-GBM), comprising a means for identifying one or more PAM pathway mutation comprising one or more nucleic acid primer, nucleic acid primer pair, nucleic acid probe, and/or an antibody specific for said mutation.

18. The kit of claim 17, wherein the PAM pathway mutation is a gain-of-function mutation that activates the PAM pathway.

19. The kit of claim 17, wherein the mutation is in PIK3CA gene.

20. The kit of claim 19, wherein the mutation is at amino acid 4, 364, 1016, 1035, or 1043 of PIK3CA protein, or at equivalent positions of homologous sequences thereto.

21. The kit of claim 20, wherein the mutation is selected from the group consisting of R4Q, G364R, F1016C, A1035V, M1043I, and M1043V.

22. The kit of claim 17, wherein the one or more primer, primer pair, probe, and/or antibodies constitute at least 10 percent of the primers, primer pairs, probes, and antibodies in the kit.

23. The kit of claim 17, further comprising a positive control.

24. The kit of claim 17, further comprising a pharmaceutical formulation for use in treating M-GBM in a subject in need thereof, comprising at least an effective amount of the PAM pathway inhibiting agent.


SEQUENCE LISTING (4 sequences)

SEQ ID NO: 1 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 2 - DNA, 9104 bp, Homo sapiens
tctccctcggcgccgccgccgccgcccgcggggctgggacccgatgcggttagagccgcggagcctggaagagccccgagcgtttctgctttgggacaaccatacatctaattccttaaagtagttttatatgtaaaacttgcaaagaatcagaacaatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaataaaatactatataatttaaataatgtaaacgcaaacagggtttgatagcacttaaactagttcatttcaaaattaagctttagaataatgcgcaatttcatgttatgccttaagtccaaaaaggtaaactttgaagattgtttgtatctttttttaaaaaacaaaacaaaacaaaaatccccaaaatatatagaaatgatggagaaggaaaaagtgatggttttttttgtcttgcaaatgttctatgttttgaaatgtggacacaacaaaggctgttattgcattaggtgtaagtaaactggagtttatgttaaattacattgattggaaaagaatgaaaatttcttatttttccattgctgttcaatttatagtttgaagtgggtttttgactgcttgtttaatgaagaaaaatgcttggggtggaagggactcttgagatttcaccagagactttttctttttaataaatcaaaccttttgatgatttgaggttttatctgcagttttggaagcagtcacaaatgagacctgttataaggtggtatttttttttttcttctggacagtatttaaaggatcttattcttatttcccagggaaattctgggctcccacaaagtaaaaaaaaaaaaaaatcatagaaaaagaatgagcaggaatagttcttattccagaattgtacagtattcaccttaagttgattttttttctccttctgcaattgaactgaatacatttttcatgcatgttttccagaaaatagaagtattaatgttattaaaaagattattttttttattaaaggctatttatattatagaaactatcattaatatatattctttatttacatgatctgtcccatagtcatgcattgttttgcaccccaaattttttattgttcatagcagcatggtcagctttcttcttgatctatagatgaggctcaggcactatcccatttataccaataaccagtgtataactacttaaggaaaacataaaaacttcatcttctttccttttatttcttatgtgaatctcccgtcttccattctcttttataattgagaatgtctcaatcatatgaaattagttaccagaattaacacaatttagactatcttcctgattccttaaacccctttactgaagtatactcatgaataatactttaaaatatgggggaatagaaaccatgaactttttacctttttaaactatttatccatatctccaaagtagaacattaaaccattttaagatatgtctcattcccaagtagtcagagctcactctccaactttattaaatactatttgagcacaggacacattcttaaacattttgaaaaacattaacccaagatgtagaggctactgctagtcgtcattctagaatctgatattttactctgtatttgaaatgaatgattaatgtcctaggaaattagctttagcagatgtccaggtgccacatcaaaaaagtgcaataattattgacagttttttagattaggcatattattggaaaacaactttataaagagtgaacattgtatactctagtaaaacagcatcactttaaaaatattcatttatgaaatctgttacctatagttgaagtcttgagtagtgaacaagggactctaataccaatactcttaatatctggctattttagatcccttaaagggcataattattggaaatttaggtatttcactaaagcatgtatataatattgccaacaagaaaagtaaatttgaagattaagggaacttacttctgcaaactgtcttgcgatagttaagcagaatttaaactctgttttaagcaggaaaccagaaagattattttgcagttgtagaagatttcataacttattaaaacttattaacattttgtgttgtttagatataggcagttgatacatactaacatcccagccttttcaatatcagggttaaattataggaaaactcagtaaaatggtacaaatctgaaagtttgatggtagaaactgaagatttaacagagaactgtgttttacccgagtgccaaaaatgctgtgagcctccttgcacaaaatttataccacttttgcatttttatctatcagtccagatagttgtctcccctccttctcccaggacctctccaccattaaaatgcacaaaccacatggccgatttcaccatttacatttattttcaaaagttactacaaccaaattaattctattagaagaaatgtagacaaattctataaagactatagattgtgacctaagaaagaaatgaggcaaagaaccaaacattgaattaaatgctacatgggtgactaagatctgtttcaagtcagtgataatatagccacttctgggtacttcagtatcagagatcagttctcgtggtttagacagttcctatctatagctgactatccttgtccttgaatatggtgtaactgactattggctctacagttttattgggccacttaagaaatatttccttgaataattattttgagaaaaagtctaaaagtaataaaaataattttaaacacactgtagtaagaaatgactgttggaaaattatgctttcactttctaccatattctcagctatacaaaaccatttattttgaagatttttagactactgttaatttgaaatctgttactcttattgtggaatttgtttttttaaaaaagatgtttctaattggatttttaaaagaagaatggaatttggttgctattttacaatagaacctaagctttttgtggttcttagtgtcctatgtaaaacttagtgtcaaagtaatcaactttgagattttcccttctattctgctttatattaaaagcccattagaaaatgggaacctggtgaatatataatgaattgtaaaatattttaatgtgtaactttttcaactgtgaaactgacttgattttttgatgaaaacagctgctgataaagtattttgtgtaaagtgtagttcttattaatcaggaaaatgatgacttgattagactgtatatgccctcttggattttattttaaatggattggtgactttcacataggtaaaacacagtccatctgtattcttttttccatcaaaaatcgagtgatttggaattataaaaaaattgtgagcagcctatttgaaaggcatcatggaaatttcacagcacaataacacggatttgttttttcttaatgatgtaaatccgtttaattcatactttgatcaatagcccatgcttgccaactctgaagaaatttaatttccagcagtattttaaagctagcctgttaactttttctgaatatttaaagttcctcttttttctatgtctgcacaaactgcagacctgggctggacccacatactcaagagtccaccttaagaaattattttgatgtccaagacatcactaaaatatttaagtttaaagataatatgtggtgttaatagattgtggtgcttttactatttaaagacaactttcatacttcagatgtttttgagaagaggggaatgtgaggggagggggcagaacagggaggagttgtttgaatgaattacattctttatatccatcctgctcatttggggcatgtctttaagagaaggctgaaagttgtgagagtatattgtataccgtaagagaatcaactcttcatcatggatgggattgtgaaggctgaactataaaattcagcattgacagcatcctcaattaataattcttggtgacagaataatacagctgggctgttttttaaaatataaacaataccatttttaattattacattaaaaattgtaaatatatctatgtgccatggcctgggaagcctgctttcttttttcataaaaattatttttactgtatgaaaagatcatggggtttagctcaaaatatctgtggtcctgataaaattggattggtaactctacctcagaaggaaaatgggaaaaaaaaatagatgagtcacaattcaatacttcaagctcagaaactgtgcagatcactgaattttagatttataaagtcagagttggcatgccttgtttttaatgatatggaagaccttaagaaaaaaacttggctgaagtttaatcgttggtccagccatttgaaaaaggcaatagtttgaggaggttcccgaattcggcatttgaaattcattttgttctctcttcttcattattagtgcatttggtgtgtgtatacttgcacacaattctgtttgtgtacacactgcttgcttagccctagtcaagaggcatcttttataaaaggtgtaaagaaatatcaaggttctaaaattcggaagagtttagaatttattaggagtttcccaagttgggatgttagtctttaaataaacttcatgcacctattccacttaaggttttgcacctcctttttattagtgcagtgccatttcttctgcttgattttaggtatgttaatattccagccttgctagttagcataaagtgacaggtgtgagccatgaggaaattttctgacttaatttgtacacaactacatataagagttttagtggaggaaaaaaattagtcccttgtgcgtatacagtagttaggtaaatgatttttctaccaacagtatactccattcctcatgtaggtaagtacagaaaaggtttttaaatgtatttttttagccagttaaagtctatgaatctatctgcaaccttatttaatctgtcactataataattttgtggttatgctaagaaccatgtatacttttaggtattcttatttttgtcaatttttctaggttggcaaggaggcagaaaaccttcattgtttcatattaaaatataattagactaaacttaattctagtatgaatttccaaaatcattatctatttatttcatttttatttaattttgtttttatttcatttttaaaagtcccttgttcaatttaacttatgttcctaagagaggttggagaacttggccttcatctgatttcaaaaatgttttgagtttcaaatgaagttaatggtttcagtgtgattcagtcctcagacctaattgggttgaataaaatctaaaagaatatacccttttggagcataacattttaataccttggggaatgtggcactaccaaaagaagactactaacacgtcagatgttcacctggaagctttatcaagaaattcgaaccacccttttggccccattaattgtagcaagtttatttctctatattttgtcattcagtgaattgaagtcctgtggtatactgcattcattagaagaaaaacgtttttaatgtccttttaatgatggcccagaaagcatttgacacagcaagatgcatgtgttactatattgagaatatagaataataacagtatcactaaatttaagacctcttcccagtcttgctgttcctagcaagaagtttggcctgtgactgcacttactgtttatgctcatcagaaactgtcaatgtctgcttttctttaactctgcagtctgtaacatcacgctgtttattaaaaaaaaaaagaaaaattaaaaaaaaaaaa

SEQ ID NO: 3 - protein, 1624 aa, Homo sapiens
MPRPGTSGRRPLLLVLLLPLFAAATSAASPSPSPSQVVEVPGVPSRPASVAVCRCCPGQTSRRSRCIRAFCRVRSCQPKKCAGPQRCLNPVPAVPSPSPSVRKRQVSLNWQPLTLQEARALLKRRRPRGPGGRGLLRRRPPQRAPAGKAPVLCPLICHNGGVCVKPDRCLCPPDFAGKFCQLHSSGARPPAPAVPGLTRSVYTMPLANHRDDEHGVASMVSVHVEHPQEASVVVHQVERVSGPWEEADAEAVARAEAAARAEAAAPYTVLAQSAPREDGYSDASGFGYCFRELRGGECASPLPGLRTQEVCCRGAGLAWGVHDCQLCSERLGNSERVSAPDGPCPTGFERVNGSCEDVDECATGGRCQHGECANTRGGYTCVCPDGFLLDSSRSSCISQHVISEAKGPCFRVLRDGGCSLPILRNITKQICCCSRVGKAWGRGCQLCPPFGSEGFREICPAGPGYHYSASDLRYNTRPLGQEPPRVSLSQPRTLPATSRPSAGFLPTHRLEPRPEPRPDPRPGPELPLPSIPAWTGPEIPESGPSSGMCQRNPQVCGPGRCISRPSGYTCACDSGFRLSPQGTRCIDVDECRRVPPPCAPGRCENSPGSFRCVCGPGFRAGPRAAECLDVDECHRVPPPCDLGRCENTPGSFLCVCPAGYQAAPHGASCQDVDECTQSPGLCGRGACKNLPGSFRCVCPAGFRGSACEEDVDECAQEPPPCGPGRCDNTAGSFHCACPAGFRSRGPGAPCQDVDECARSPPPCTYGRCENTEGSFQCVCPMGFQPNTAGSECEDVDECENHLACPGQECVNSPGSFQCRTCPSGHHLHRGRCTDVDECSSGAPPCGPHGHCTNTEGSFRCSCAPGYRAPSGRPGPCADVNECLEGDFCFPHGECLNTDGSFACTCAPGYRPGPRGASCLDVDECSEEDLCQSGICTNTDGSFECICPPGHRAGPDLASCLDVDECRERGPALCGSQRCENSPGSYRCVRDCDPGYHAGPEGTCDDVDECQEYGPEICGAQRCENTPGSYRCTPACDPGYQPTPGGGCQDVDECRNRSFCGAHAVCQNLPGSFQCLCDQGYEGARDGRHCVDVNECETLQGVCGAALCENVEGSFLCVCPNSPEEFDPMTGRCVPPRTSAGTFPGSQPQAPASPVLPARPPPPPLPRRPSTPRQGPVGSGRRECYFDTAAPDACDNILARNVTWQECCCTVGEGWGSGCRIQQCPGTETAEYQSLCPHGRGYLAPSGDLSLRRDVDECQLFRDQVCKSGVCVNTAPGYSCYCSNGYYYHTQRLECIDNDECADEEPACEGGRCVNTVGSYHCTCEPPLVLDGSQRRCVSNESQSLDDNLGVCWQEVGADLVCSHPRLDRQATYTECCCLYGEAWGMDCALCPAQDSDDFEALCNVLRPPAYSPPRPGGFGLPYEYGPDLGPPYQGLPYGPELYPPPALPYDPYPPPPGPFARREAPYGAPRFDMPDFEDDGGPYGESEAPAPPGPGTRWPYRSRDTRRSFPEPEEPPEGGSYAGSLAEPYEELEAEECGILDGCTNGRCVRVPEGFTCRCFDGYRLDMTRMACVDINECDEAEAASPLCVNARCLNTDGSFRCICRPGFAPTHQPHHCAPARPRA

SEQ ID NO: 4 - DNA, 5163 bp, Homo sapiens
atgccgaggcctggcaccagcggccgccgccccctcctgctggtgctgttgctgccgctcttcgcagccgccacctccgccgccagccccagccccagccccagccaggtcgtcgaggtcccgggggtccccagccgcccggccagcgttgctgtttgtcgctgctgcccgggccagacgtctaggaggagccgctgcatccgagccttctgcagggtccgaagctgccagcccaaaaagtgtgcaggcccccagcggtgcctgaacccagtgcctgcagtgcccagtcccagccccagcgtgaggaagagacaggtgtccctcaactggcagccactgacgctccaggaggccagagctctactgaagcggcggcggccccgggggccagggggccggggactactgagaaggaggcccccacagcgtgcccccgctggcaaggccccggtcctgtgtcccttgatctgtcacaatggcggtgtgtgcgtgaagcctgaccgctgcctctgtcccccggacttcgctggcaagttctgccagttgcactcctcgggcgcccggcccccggccccggctgtaccaggcctcacccgctccgtgtacactatgccactggccaaccaccgcgacgacgagcacggcgtggcatctatggtgagcgtccacgtggagcacccgcaggaggcgtcggtggtggtgcaccaggtggagcgtgtgtctggcccttgggaggaggcggacgctgaggcggtggcgcgggcggaagcggcggcgcgggcggaggcggcagcgccctacacggtgttggcacagagcgcgccgcgggaggacggctactcagatgcctcgggcttcggttactgctttcgggagctgcgcggaggcgaatgcgcgtccccgctgcccgggctccggacgcaggaggtctgctgccgaggggccggcttggcctggggcgttcacgactgtcagctgtgctccgagcgcctggggaactccgaaagagtgagcgccccagatggaccttgtccaaccggctttgaaagagttaatgggtcctgcgaagatgtggatgagtgcgcgactggcgggcgctgccagcacggcgagtgtgcaaacacgcgcggcgggtacacgtgtgtgtgccccgacggctttctgctcgactcgtcccgcagcagctgcatctcccaacacgtgatctcagaggccaaagggccctgcttccgcgtgctccgcgacggcggctgttcgctgcccattctgcggaacatcactaaacagatctgctgctgcagccgcgtaggcaaggcctggggccggggctgccagctctgcccacccttcggctcagagggtttccgggagatctgcccggctggtcctggttaccactactcggcctccgacctccgctacaacaccagacccctgggccaggagccaccccgagtgtcactcagccagcctcgtaccctgccagccacctctcggccatctgcaggctttctgcccacccatcgcctggagccccggcctgaaccccggcccgatccccggcccggccctgagcttcccttgcccagcatccctgcctggactggtcctgagattcctgaatcaggtccctcctccggcatgtgtcagcgcaacccccaggtctgcggcccaggacgctgcatttcccggcccagcggctacacctgcgcttgcgactctggcttccggctcagcccccagggcacccgatgcattgatgtggacgaatgtcgccgcgtgcccccgccctgtgctcccgggcgctgcgagaactcaccaggcagcttccgctgcgtgtgcggcccgggcttccgagccggcccacgggctgcggaatgcctggatgtggacgagtgccaccgcgtgccgccgccgtgtgacctcgggcgctgcgagaacacgccaggcagcttcctgtgcgtgtgccccgccgggtaccaggctgcaccgcacggagccagctgccaggatgtggatgaatgcacccagagcccaggcctgtgtggccgaggggcctgcaagaacctgcctggctctttccgctgtgtttgcccggctggcttccggggctcggcgtgtgaagaggatgtggatgagtgtgcccaggagccgccgccctgtgggcccggccgctgtgacaacacggcaggctcctttcactgtgcctgccctgctggcttccgctcccgagggcccggggccccctgccaagatgtggatgagtgtgcccgaagccccccaccctgcacctacggccggtgtgagaacacagaaggcagcttccagtgtgtctgccccatgggcttccaacccaacactgctggctccgagtgcgaggatgtggatgagtgtgagaaccacctcgcatgccctgggcaggagtgtgtgaactcgcccggctccttccagtgcaggacctgtccttctggccaccacctgcaccgtggcagatgcactgatgtggacgaatgcagttcgggtgcccctccctgtggtccccacggccactgcactaacaccgaaggctccttccgctgcagctgcgcgccaggctaccgggcgccgtcgggtcggcccgggccctgcgcagacgtgaacgagtgcctggagggcgatttctgcttccctcacggcgagtgcctcaacactgacggctcctttgcctgtacttgtgcccctggctaccgacccggaccccgcggagcctcttgcctcgacgttgacgagtgcagcgaggaggacctttgccagagcggcatctgtaccaacaccgacggctccttcgagtgcatctgtcctccgggacaccgcgctggcccggacctcgcctcctgcctcgacgtggacgaatgtcgcgagcgaggcccagccctgtgcgggtcgcagcgctgtgagaactctcccggctcctaccgctgtgtccgggactgcgatcctgggtaccacgcgggccccgagggcacctgtgacgatgtggatgagtgccaagaatatggtcccgagatttgtggagcccagcgttgtgagaacacccctggctcctaccgctgcacaccagcctgtgaccctggctatcagcccacgccagggggcggatgccaggatgtggacgaatgccggaaccggtccttctgcggtgcccacgccgtgtgccagaacctgcccggctccttccagtgcctctgtgaccagggttacgagggggcacgggatgggcgtcactgcgtggatgtgaacgagtgtgaaacactacagggtgtatgtggagctgccctgtgtgaaaatgtcgaaggctccttcctctgtgtctgccccaacagcccggaagagtttgaccccatgactggacgctgtgttcccccacgaacttctgctggcacgttcccaggctcgcagccccaggcacctgctagccccgttctgcccgccaggccacctccgccacccctgccccgccgacccagcacacctaggcagggccctgtggggagtgggcgccgggagtgctactttgacacagcggccccggatgcatgtgacaacatcctggctcggaatgtgacatggcaggagtgctgctgtactgtgggtgagggctggggcagcggctgccgcatccagcagtgcccgggcaccgagacagctgagtaccagtcattgtgccctcacggccggggctacctggcgcccagtggagacctgagcctccggagagacgtggacgaatgtcagctcttccgagaccaggtgtgcaagagtggcgtgtgtgtgaacacggccccgggctactcatgctattgcagcaacggctactactaccacacacagcggctggagtgcatcgacaatgacgagtgcgccgatgaggaaccggcctgtgagggcggccgctgtgtcaacactgtgggctcttatcactgtacctgcgagcccccactggtgctggatggctcgcagcgccgctgcgtctccaacgagagccagagcctcgatgacaatctgggagtgtgctggcaggaagtgggggctgacctcgtgtgcagccaccctcggctggaccgtcaggccacctacacagagtgctgctgcctgtatggagaggcctggggcatggactgcgccctctgccctgcgcaggactcagatgacttcgaggccctgtgcaatgtgctacgcccccccgcatatagccccccgcgaccaggtggctttggactcccctacgagtacggcccagacttaggtccaccttaccagggcctcccatatgggcctgagttgtacccaccacctgcgctaccctacgacccctacccaccgccacctgggcccttcgcccgccgggaggctccttatggggcaccccgcttcgacatgccagactttgaggacgatggtggcccctatggcgaatctgaggctcctgcgccacctggcccgggcacccgctggccctatcggtcccgggacacccgccgctccttcccagagcccgaggagcctcctgaaggtggaagctatgctggttccctggctgagccctacgaggagctggaggcggaggagtgcgggatcctggacggctgcaccaacggccgctgcgtgcgcgtccccgaaggcttcacctgccgttgcttcgacggctaccgcctggacatgacccgcatggcctgcgttgacatcaacgagtgtgatgaggccgaggctgcctccccgctgtgcgtcaacgcgcgttgcctcaacacggatggctccttccgctgcatctgccgcccgggattcgcacccacgcaccagccgcaccactgtgcgcccgcacggccccgggcctgagccctggcacccgctggccgcccacccgcgcccgccactcggggcccctgccgcgcatcctgcagcccgcttatgcgtatgtgcacggggccgcccgcctggacctggagaagggacctacggacgcctggaagctgcgacgccctgcactgctcccgcctccaccagcgcctcccactgatgtcgtggtcccgggcctggcccaggggcccctttacatgccctctcccttttataaaattttccattaaaaaccacctattttctatcaaaaaaaaaaaaaaaaaa